(19) United States
(12) Patent Application Publication
(10) Pub. No.: US 2024/0337668 A1
(43) Pub. Date: Oct. 10, 2024

(54) USE OF BIOMARKERS TO DETERMINE WHETHER A SUBJECT HAS SUSTAINED, MAY HAVE SUSTAINED OR IS SUSPECTED OF SUSTAINING A SUBACUTE ACQUIRED BRAIN INJURY (ABI)

(71) Applicant: Abbott Laboratories, Abbott Park, IL (US)

(72) Inventors: Beth McQuiston, Abbott Park, IL (US); Saul Datwyler, Abbott Park, IL (US); Raj Chandran, Abbott Park, IL (US); Jaime Marino, Abbott Park, IL (US)

(21) Appl. No.: 18/632,426

(22) Filed: Apr. 11, 2024

Related U.S. Application Data
(63) Continuation of application No. PCT/US24/22929, filed on Apr. 4, 2024.
(60) Provisional application No. 63/456,818, filed on Apr. 4, 2023.

(51) Int. Cl. G01N 33/68

(52) U.S. Cl. CPC G01N 33/6896; G01N 2333/435; G01N 2333/948; G01N 2800/28

(57) ABSTRACT

Disclosed herein are methods that aid in the determination of whether a subject has or may have sustained an acquired brain injury, such as a traumatic brain injury (TBI), by detecting levels of at least one biomarker, glial fibrillary acidic protein (GFAP), in one or more samples taken from a subject, such as a human subject.

### RELATED APPLICATION INFORMATION

This application is a continuation of International Application No. PCT/US24/22929, filed Apr. 4, 2024, which claims priority to U.S. Application No. 63/456,818, filed on Apr. 4, 2023, the contents of which are herein incorporated by reference.

### SEQUENCE LISTING STATEMENT

The contents of the electronic sequence listing titled ABBTL-41876.601-ST26.xml (Size: 7917 bytes; and Date of Creation: Apr. 4, 2024) is herein incorporated by reference in its entirety.

### TECHNICAL FIELD

The present disclosure relates to methods of aiding in the diagnosis and evaluation of a subject that has sustained, may have sustained, or is suspected of sustaining an acquired brain injury (ABI), such as a traumatic brain injury (TBI). The methods involve detecting levels of at least one biomarker, such as glial fibrillary acidic protein (GFAP), ubiquitin carboxy-terminal hydrolase L1 (UCH-L1), or a combination thereof, in one or more samples taken from a subject at one or more time points from within after about 24 hours to about 3 weeks after the subject has sustained, may have sustained, or is suspected of sustaining an injury to the head, and determining whether a subject (a) has not suffered or recovered from an actual ABI; (b) should be monitored for recovery from an actual ABI; or (c) sustained and ABI and is not recovering from the actual ABI based on whether the levels of the one or more biomarkers in the sample are lower, higher, or within the range of a certain reference level.

### BACKGROUND

More than 5 million mild traumatic brain injuries (TBIs) occur each year in the United States alone. Currently, there is no simple, objective, accurate measurement available to help in patient assessment. In fact, much of TBI evaluation and diagnosis is based on subjective data. Unfortunately, objective measurements such as head CT and Glasgow Coma Scale (GCS) score are not very comprehensive or sensitive in evaluating mild TBI. Moreover, head CT is unrevealing a vast majority of the time for mild TBI, is expensive, and exposes the patient to unnecessary radiation. Additionally, a negative head CT does not mean the patient has been cleared from having a concussion; rather it just means certain interventions, such as surgery, are not warranted. Clinicians and patients need objective, reliable information to accurately evaluate this condition to promote appropriate triage and recovery. To date, limited data have been available for the use of UCH-L1 and GFAP in the acute care setting to aid in patient evaluation and management. To date, limited data have been available for the use of UCH-L1 and GFAP in the acute care setting to aid in patient evaluation and management.

Mild TBI or concussion is much harder to objectively detect and presents an everyday challenge in emergency care units globally. Concussion usually causes no gross pathology, such as hemorrhage, and no abnormalities on conventional computed tomography scans of the brain, but rather rapid-onset neuronal dysfunction that resolves in a spontaneous manner over a few days to a few weeks. Approximately 15% of mild TBI patients suffer persisting cognitive dysfunction. There is an unmet need for detecting and assessing mild TBI victims on scene, in emergency rooms and clinics, in the sports area and in military activity (e.g., combat).

Current algorithms for assessment of the severity of brain injury include Glasgow Coma Scale score and other measures. These measures may at times be adequate for relating acute severity but are insufficiently sensitive for subtle pathology which can result in persistent deficit. GCS and other measures also do not enable differentiation among types of injury and may not be adequate. Thus, patients grouped into a single GCS level entering a clinical trial may have vastly heterogeneous severity and type of injury. Because outcomes also vary accordingly, inappropriate classification undermines the integrity of a clinical trial. Improved classification of injury will enable more precise delineation of disease severity and type for TBI patients in clinical trials.

Additionally, current brain injury trials rely on outcome measures such as Glasgow Outcome Scale Extended, which capture global phenomena but fail to assess for subtle differences in outcome. Sensitive outcome measures are needed to determine how well patients have recovered from brain injury in order to test therapeutics and prophylactics.

### SUMMARY

In one aspect, the present disclosure relates to a method for aiding in the diagnosis and evaluation of a subject that has sustained or may have sustained an acquired brain injury (ABI).

The method comprises:
a. obtaining a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI; and
b. determining that the subject:
i) has not suffered or has recovered from an actual ABI if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

In still other aspects of the above method:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects of the above method, the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

In still further aspects of the above method:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Moreover, in some aspects of the above, (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

In still further aspects of the above method:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL, and further wherein the GFAP level, the UCH-L1 level.

Moreover, in some aspects of the above, (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

In yet further aspects of the above method:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Moreover, in some aspects of the above, (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

In still other aspects of the above method, measuring the GFAP level, UCH-L1 level, or GFAP level and UCH-L1 level is done by immunoassay or a clinical chemistry assay.

In still further aspects of the above method, the assay is performed using a point-of-care assay or single molecule detection.

In still further aspects of the above method, the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

In still yet further aspects of the above method, the actual or suspected ABI is caused by physical shaking, blunt impact by an external mechanical or other force that results in a closed or open head trauma, one or more falls, explosions or blasts or other types of blunt force trauma. Specifically, in some aspects, the actual or suspected ABI is a traumatic brain injury.

In yet further aspects, the actual or suspected ABI is a subacute ABI. In other aspects, when the actual or suspected ABI is a traumatic brain injury, the traumatic brain injury is a subacute traumatic brain injury.

In yet further aspects of the above method, the actual or suspected ABI is caused by fire exposure, ingestion of a chemical or toxin, or a combination of fire exposure, and ingestion of a chemical or toxin. Specifically, the chemical or toxin is mold, asbestos, a pesticide, an insecticide, an organic solvent, a paint, a glue, a gas, an organic metal, a drug of abuse or one or more combinations thereof.

In still further aspects of the above method, the actual or suspected ABI is caused by an autoimmune disease, a metabolic disorder, a brain tumor, hypoxia, a viral infection, a fungal infection, a bacterial infection, meningitis, hydrocephalus, or any combinations thereof. In yet further aspects of the above method, the blood sample is whole blood, serum or plasma, either venous blood or capillary blood.

In yet further aspects, the blood sample is obtained from venous blood and is whole blood, serum, or plasma. In still yet other aspects, blood sample is obtained from capillary blood and is whole blood, serum, or plasma. In yet further aspects, the blood sample is obtained from venous blood and is whole blood, serum, or plasma. In still yet other aspects, blood sample is obtained from capillary blood and is whole blood, serum, or plasma. In some aspects blood sample is obtained from venous blood and is whole blood. In still further aspects, the blood sample is obtained from venous blood and is serum. In still further aspects, the blood sample is obtained from venous blood and is plasma. In some aspects blood sample is obtained from venous blood and is whole blood. In still further aspects, the blood sample is obtained from venous blood and is serum. In still further aspects, the blood sample is obtained from venous blood and is plasma.

In still further aspects of the above method, the subject is a human subject.

In still another aspect, the method comprises:
a. performing one or more assays on at least one or more samples obtained from the subject within after twenty-four hours to (i) about one week, (ii) about two weeks, or (iii) about three weeks, after an actual or suspected ABI to measure or detect a level of a biomarker in the sample, said biomarker comprising glial fibrillary acidic protein (GFAP), ubiquitin carboxy-terminal hydrolase L1 (UCH-L1), or a combination of GFAP and UCH-L1;
b. determining that the subject:
i) has not suffered or recovered from an actual ABI if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

In still other aspects of the above method:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects of the above method, the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

In still further aspects of the above method:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Moreover, in some aspects of the above, (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

In still further aspects of the above method:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL, and further wherein the GFAP level, the UCH-L1 level.

Moreover, in some aspects of the above, (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

In yet further aspects of the above method:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Moreover, in some aspects of the above, (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

In still other aspects of the above method, measuring the GFAP level, UCH-L1 level, or GFAP level and UCH-L1 level is done by immunoassay or a clinical chemistry assay.

In still further aspects of the above method, the assay is performed using a point-of-care assay or single molecule detection.

In still further aspects of the above method, the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

In still yet further aspects of the above method, the actual or suspected ABI is caused by physical shaking, blunt impact by an external mechanical or other force that results in a closed or open head trauma, one or more falls, explosions or blasts or other types of blunt force trauma. Specifically, in some aspects, the actual or suspected ABI is a traumatic brain injury. In yet further aspects, the actual or suspected ABI is a subacute ABI. In other aspects, when the actual or suspected ABI is a traumatic brain injury, the traumatic brain injury is a subacute traumatic brain injury.

In yet further aspects of the above method, the actual or suspected ABI is caused by fire exposure, ingestion of a chemical or toxin, or a combination of fire exposure, and ingestion of a chemical or toxin. Specifically, the chemical or toxin is mold, asbestos, a pesticide, an insecticide, an organic solvent, a paint, a glue, a gas, an organic metal, a drug of abuse or one or more combinations thereof.

In still further aspects of the above method, the actual or suspected ABI is caused by an autoimmune disease, a metabolic disorder, a brain tumor, hypoxia, a viral infection, a fungal infection, a bacterial infection, meningitis, hydrocephalus, or any combinations thereof. In yet further aspects of the above method, the blood sample is whole blood, serum or plasma, either venous blood or capillary blood. In yet further aspects, the blood sample is obtained from venous blood and is whole blood, serum, or plasma. In still yet other aspects, blood sample is obtained from capillary blood and is whole blood, serum, or plasma. In some aspects blood sample is obtained from venous blood and is whole blood. In still further aspects, the blood sample is obtained from venous blood and is serum. In still further aspects, the blood sample is obtained from venous blood and is plasma. In some aspects blood sample is obtained from venous blood and is whole blood. In still further aspects, the blood sample is obtained from venous blood and is serum. In still further aspects, the blood sample is obtained from venous blood and is plasma.

In still further aspects of the above method, the subject is a human subject. In another aspect, the present disclosure relates to a system. The system comprises:
an apparatus comprising:
a storage device having machine-readable instructions stored thereon; and
a processor to at least one of instantiate or execute the machine-readable instructions to:
identify a (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b); and
instructions for determining that the subject:
i) has not suffered or has recovered from an actual acquired brain injury (ABI) if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

In some aspects of the above system, the apparatus is a point-of-care device or a non-point-of-care device.

In still yet another aspect, the present disclosure relates to an apparatus. The apparatus can comprise:
a storage device having machine-readable instructions stored on the storage device; and
a processor to at least one of instantiate or execute the machine-readable instructions to:
identify (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from the apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

In some aspects, the apparatus is a point-of-care device or a non-point-of-care device.

In yet still further aspects, the present disclosure relates to a non-transitory machine-readable storage medium. The non-transitory machine-readable storage medium can comprise instructions to cause one or more processors to at least:
identify (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from an apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

In some aspects of the non-transitory machine-readable storage medium, the apparatus is a point-of-care device or a non-point of care device.

### DETAILED DESCRIPTION

The present disclosure relates to methods of aiding in the diagnosis and evaluation of a subject that has sustained, may have sustained, or is suspected of sustaining an acquired brain injury (ABI), such as a traumatic brain injury (TBI). The methods involve detecting levels of at least one biomarker, such as glial fibrillary acidic protein (GFAP), ubiquitin carboxy-terminal hydrolase L1 (UCH-L1), or a combination thereof, in one or more samples taken from a subject at one or more time points within after twenty-four hours to (i) about one week, (ii) about two weeks, or (iii) about three weeks after the subject has sustained, may have sustained, or is suspected of sustaining an injury to the head, and determining whether a subject (a) has not suffered or recovered from an actual ABI based on whether the levels of one or more biomarkers are lower than a reference level; (b) should be monitored for recovery from an actual ABI based on whether the levels of one or more biomarkers are within a range of a reference level; or (c) sustained and ABI and is not recovering from the actual ABI based on whether the levels of the one or more biomarkers are greater than a reference level. As used herein, “has not suffered or recovered” means the subject either did not sustain an ABI, or did sustain and recovered from an ABI.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Acquired brain injury” or (ABI) as used herein refers to damage to the brain that is caused by events occurring after birth. In other words, acquired brain injuries are not genetic or congenital but are the result neurological conditions and injuries. Acquired brain injuries are often divided into two categories. The first category is acquired traumatic brain injuries (TBI), that occur due to an external force, such as, for example, a sports injury, fall, physical shaking, blunt force trauma, explosion, blast, or exposure to a fire. The second category is non-traumatic acquired brain injuries that in some cases are caused by internal factors and include stroke, tumors, anoxia, infections, metabolic disorders, and others. As used herein, an acquired brain injury does not include or encompass damage to the brain that is caused by a stroke (including, such as, for example, ischemic stroke, hemorrhagic stroke, or a transient ischemic attack, etc.).

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

An “analog assay” as used herein refers to an assay in which the presence of and/or concentration of an analyte in a test sample is determined by measuring the total signal produced (e.g., fluorescence, color, etc.) by the analyte in an entire reaction mixture (e.g., a single reaction vessel). In an analog assay, the noise is indistinguishable from the signal. An example of an analog assay is an assay in which the presence of and/or concentration of an analyte is determined by measuring the total signal produced from a plurality of beads or microparticles contained in a single reaction vessel.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F (ab′) fragments, F (ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-UCH-L1 antibody or a UCH-L1 antibody).

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Aptamer” as used herein refers to a nucleic acid that has a specific binding affinity for a target analyte or molecule. It is recognized that affinity interactions are a matter of degree; however, in this context, the “specific binding affinity” of an aptamer for its analyte or target means that the aptamer binds to its analyte or target generally with a much higher degree of affinity than it binds to other components in a biological sample. An “aptamer” is a set of copies of one type or species of nucleic acid molecule that comprises a particular nucleotide sequence. An aptamer can include any suitable number of nucleotides, including any number of chemically modified nucleotides. “Aptamers” refers to more than one such set of molecules.

Different aptamers can have either the same or different numbers of nucleotides. Aptamers can be DNA or RNA or chemically modified nucleic acids and can be single-stranded, double-stranded, or contain double-stranded regions, and can include higher ordered structures. An aptamer can also be a photoaptamer, where a photoreactive or chemically reactive functional group is included in the aptamer to allow it to be covalently linked to its corresponding analyte or target. In some aspects, an aptamer may include a detectable label.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

A “clinically-relevant time frame” refers to a time frame (e.g., seconds, minutes, or hours) during which a careful and prudent medical practitioner (e.g., doctor, nurse, paramedic, or other) would reasonably consider the results of one or more biomarker tests to have bearing on an imaging procedure, such as a head CT scan, or pursuant to guidelines established by an overseeing entity (e.g., a standards-setting body such as the World Health Organization (WHO), physicians review board, regulatory approval authority such as FDA, EMEA or other, etc.).

“Communicated” or “communicating” as used herein refers to the conveying, transmitting and/or reporting of an item of information. In some aspects, the information that is communicated is an item of information obtained by performing an assay, such as, the amount or presence of a biomarker in a sample (e.g., a result). The information obtained by performing an assay can be communicated by a computer, in a document and/or spreadsheet, on a mobile device (e.g., a smart phone), on a website, in an e-mail, or any combination thereof. In some other aspects, information is communicated on or from an apparatus, device, or instrument. In other aspects, the information is communicated by being displayed, such as on an apparatus, device, or instrument.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Correlated to” as used herein refers to compared to.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

A head CT scan is “negative” for a TBI when no intracranial lesion(s) is observed in an image taken from a subject that has sustained, may have sustained or is suspected of sustaining an injury to the head. To further clarify, the head CT scan of a subject is “negative” for a TBI when a lesion is not found or identified; however, in some aspects, the subject may still be experiencing symptoms (e.g., of TBI) even though the head CT is negative. Most subjects will be negative for a TBI on head CT given that not all injuries or lesions can be visualized by head CT. Consequently, the methods and assays described herein can be used to provide an assessment or determination of a subject with a negative head CT that may still have a TBI.

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from human subjects known to have sustained an injury to the head (and more particularly, samples obtained from human subjects known to have sustained a (i) mild TBI; and/or (ii) moderate, severe, or moderate-to-severe TBI and samples obtained from human subjects known not to have sustained an injury to the head may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the present disclosure. It is well within the ordinary skill of one in the art to determine the sensitivity and specificity associated with a given reference level in the methods of the present disclosure, for example by repeated statistical analysis of assay data using a plurality of different possible reference levels.

Practically, when discriminating between a subject as having a traumatic brain injury or not having a traumatic brain injury or a subject as having a mild versus a moderate, severe, or moderate-to-severe traumatic brain injury, the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting traumatic brain injury or determining a mild versus moderate, severe, or moderate-to-severe traumatic brain injury will be readily apparent to those skilled in the art. In discriminating whether a subject has or does not have a traumatic brain injury or a mild versus a moderate, severe, or moderate-to-severe traumatic brain injury, the higher the cutoff, specificity improves as more true negatives (i.e., subjects not having a traumatic brain injury, not having a mild traumatic brain injury, not have a moderate traumatic brain injury, not having a severe traumatic brain injury or not having a moderate-to-severe traumatic brain injury) are distinguished from those having a traumatic brain injury, a mild traumatic brain injury, a moderate traumatic brain injury, a severe traumatic brain injury or a moderate-to-severe traumatic brain injury. But at the same time, raising the cutoff decreases the number of cases identified as positive overall, as well as the number of true positives, so the sensitivity must decrease. Conversely, the lower the cutoff, sensitivity improves as more true positives (i.e., subjects having a traumatic brain injury, having a mild traumatic brain injury, having a moderate traumatic brain injury, having a severe traumatic brain injury or having a moderate-to-severe traumatic brain injury) are distinguished from those who do not have a traumatic brain injury, a mild traumatic brain injury, a moderate traumatic brain injury, a severe traumatic brain injury or a moderate-to-severe traumatic brain injury. But at the same time, lowering the cutoff increases the number of cases identified as positive overall, as well as the number of false positives, so the specificity must decrease.

Generally, a high sensitivity value helps one of skill rule out disease or condition (such as a traumatic brain injury, mild traumatic brain injury, moderate traumatic brain injury, severe traumatic brain injury or moderate-to-severe traumatic brain injury), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physician or practitioner can choose.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Digital assay” as used herein refers to an assay in which an analyte is captured and a molecule of the analyte segregated and interrogated (e.g., to detect the presence and/or concentration of the analyte in a sample). In a digital assay, noise is separated from signal. In a digital assay, the results are assigned a value of 1 or 0. Examples of digital assays include one or more of the following (which may overlap but are not mutually exclusive): single molecule detection assay, a nanowell assay, a single molecule enzyme linked immunosorbent assay, a direct capture counting assay, etc.

“Drugs of abuse” is used herein to refer to one or more additive substances (such as a drug) taken for non-medical reasons (such as for, example, recreational and/or mind-altering effects). Excessive overindulgence, use or dependence of such drugs of abuse is often referred to as “substance abuse”. Examples of drugs of abuse include alcohol, barbiturates, benzodiazepines, cannabis, cocaine, hallucinogens (such as ketamine, mescaline (peyote), PCP, psilocybin, DMT and/or LSD), methaqualone, opioids, amphetamines (including methamphetamines), anabolic steroids, inhalants (namely, substances which contain volatile substances that contain psychoactive properties such as, for example, nitrites, spray paints, cleaning fluids, markers, glues, etc.) and combinations thereof.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25:1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of UCH-L1, GFAP, or UCH-L1 and GFAP. Non-limiting examples of a DVD-Ig binding protein include (1) a DVD-Ig binding protein that binds one or more epitopes of UCH-L1, a DVD-Ig binding protein that binds an epitope of a human UCH-L1 and an epitope of UCH-L1 of another species (for example, mouse), and a DVD-Ig binding protein that binds an epitope of a human UCH-L1 and an epitope of another target molecule; (2) a DVD-Ig binding protein that binds one or more epitopes of GFAP, a DVD-Ig binding protein that binds an epitope of a human GFAP and an epitope of GFAP of another species (for example, mouse), and a DVD-Ig binding protein that binds an epitope of a human GFAP and an epitope of another target molecule; or (3) a DVD-Ig binding protein that binds one or more epitopes of UCH-L1 and GFAP, a DVD-Ig binding protein that binds an epitope of a human UCH-L1, a human GFAP, and an epitope of UCH-L1 of another species (for example, mouse), and a DVD-Ig binding protein that binds an epitope of a human UCH-L1, a human GFAP, and an epitope of another target molecule.

“Dynamic range” as used herein refers to range over which an assay readout is proportional to the amount of target molecule or analyte in the sample being analyzed.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F (ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpc.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans and by GFAP gene counterparts in other species, and which can be produced (e.g., by recombinant means, in other species).

“GFAP status” can mean either the level or amount of GFAP at a point in time (such as with a single measure of GFAP), the level or amount of GFAP associated with monitoring (such as with a repeat test on a subject to identify an increase or decrease in GFAP amount), the level or amount of GFAP associated with treatment for traumatic brain injury (whether a primary brain injury and/or a secondary brain injury) or combinations thereof.

“Glasgow Coma Scale” or “GCS” as used herein refers to a 15-point scale (e.g., described in 1974 by Graham Teasdale and Bryan Jennett, Lancet 1974; 2:81-4) that provides a practical method for assessing impairment of conscious level in patients who have suffered a brain injury. The test measures the best motor response, verbal response and eye opening response with these values: I. Best Motor Response (6—obey 2-part request; 5—brings hand above clavicle to stimulus on head neck; 4—bends arm at elbow rapidly but features not predominantly abnormal; 3—bends arm at elbow, features clearly predominantly abnormal; 2—extends arm at elbow; 1—no movement in arms/legs, no interfering factor; NT—paralyzed or other limiting factor); II. Verbal Response (5—correctly gives name, place and date; 4—not orientated but communication coherently; 3—intelligible single words; 2—only moans/groans; 1—no audible response, no interfering factor; NT-factor interfering with communication); and III. Eye Opening (4—open before stimulus; 3—after spoken or shouted request; 2—after fingertip stimulus; 1—no opening at any time, no interfering factor; NT-closed by local factor). The final score is determined by adding the values of I+II+III. A subject is considered to have a mild TBI if the GCS score is 13-15. A subject is considered to have a moderate TBI if the GCS score is 9-12. A subject is considered to have a severe TBI if the GCS score is 8 or less, typically 3-8.
“Glasgow Outcome Scale” as used herein refers to a global scale for functional outcome that rates patient status into one of five categories: Dead, Vegetative State, Severe Disability, Moderate Disability or Good Recovery. “Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative state | VX | Condition of unawareness with only reflex |
| | | | responses but with periods of spontaneous eye |
| | | | opening |
| 3 | Lower severe disability | SD− | Patient who is dependent for daily support for |
| 4 | Upper severe disability | SD+ | mental or physical disability, usually a |
| | | | combination of both. If the patient can be left |
| | | | alone for more than 8 hours at home it is upper |
| | | | level of SD, if not then it is low level of SD. |
| 5 | Lower moderate disability | MD− | Patients have some disability such as aphasia, |
| 6 | Upper moderate disability | MD+ | hemiparesis or epilepsy and/or deficits of |
| | | | memory or personality but are able to look after |
| | | | themselves. They are independent at home but |
| | | | dependent outside. If they are able to return to |
| | | | work even with special arrangement it is upper |
| | | | level of MD, if not then it is low level of MD. |
| 7 | Lower good recovery | GR− | Resumption of normal life with the capacity to |
| 8 | Upper good recovery | GR+ | work even if pre-injury status has not been |
| | | | achieved. Some patients have minor neurological |
| | | | or psychological deficits. If these deficits are |
| | | | not disabling then it is upper level of GR, if |
| | | | disabling then it is lower level of GR. |

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Injury to the head” or “head injury” as used interchangeably herein, refers to any trauma to the scalp, skull, or brain. Such injuries may include only a minor bump on the skull or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue. The head injury can be either traumatic or non-traumatic.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), one or more falls (e.g., as in sports or other activities), explosions or blasts (collectively, “blast injuries”) and by other types of blunt force trauma. Such injuries typically are considered traumatic brain injuries, TBIs. Alternatively, an injury to the head may be caused by the ingestion and/or exposure to a fire, chemical, toxin or a combination of a chemical and toxin. Examples of such chemicals and/or toxins include molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, hypoxia, a viral infection (e.g., SARS-COV-2), a fungal infection, a bacterial infection, meningitis, hydrocephalus, or any combinations thereof. Such injuries typically are considered non-traumatic brain injuries, or non-TBIs.

In some cases, it is not possible to be certain whether any such event or injury has occurred or taken place. For example, there may be no history on a patient or subject, the subject may be unable to speak, the subject may be aware of what events they were exposed to, etc. Such circumstances are described herein as the subject “may have sustained an injury to the head.” As described herein, the head injury does not include and specifically excludes a cerebral vascular accident, such as stroke.

“Intracranial lesion” as used herein refers to an area of injury within the brain. An intracranial lesion can be an abnormality seen on a CT scan or brain-imaging test, such as magnetic resonance imaging (MRI). On CT or MRI scans, brain lesions can appear as dark or light spots that do not look like normal brain tissue.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111 In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5 (6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N. Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (scc, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16:1324-1328 (2006); Adamczyk et al., Bioorg. Med. Chem. Lett. 4:2313-2317 (2004); Adamczyk et al., Biorg. Med. Chem. Lett. 14:3917-3921 (2004); and Adamczyk et al., Org. Lett. 5:3779-3782 (2003)).

In one aspect, the acridinium compound is an acridinium-9-carboxamide. Methods for preparing acridinium 9-carboxamides are described in Mattingly, J. Biolumin. Chemilumin. 6:107-114 (1991); Adamczyk et al., J. Org. Chem. 63:5636-5639 (1998); Adamczyk et al., Tetrahedron 55:10899-10914 (1999); Adamczyk et al., Org. Lett. 1:779-781 (1999); Adamczyk et al., Bioconjugate Chem. 11:714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5:3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl) acridinium fluorosulfonate (available from Cayman Chemical, Ann Arbor, MI). Methods for preparing acridinium 9-carboxylate aryl esters are described in McCapra et al., Photochem. Photobiol. 4:1111-21 (1965); Razavi et al., Luminescence 15:245-249 (2000); Razavi et al., Luminescence 15:239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6X His tag, which has an amino acid sequence of HHHHHH (SEQ ID NO: 3), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 4) and derivatives thereof (e.g., ADDDDK (SEQ ID NO: 5), etc.); (iii) Miscellaneous sequences can be used to link or connect the light and/or heavy chain variable regions of single chain variable region fragments. Examples of other linking sequences can be found in Bird et al., Science 242:423-426 (1988); Huston et al., PNAS USA 85:5879-5883 (1988); and McCafferty et al., Nature 348:552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Magnetic resonance imaging” or “MRI” as used interchangeably herein refers to a medical imaging technique used in radiology to form pictures of the anatomy and the physiological processes of the body in both health and disease (e.g., referred to herein interchangeably as “an MRI”, “an MRI procedure” or “an MRI scan”). MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen (e.g., although cross-reactivity or shared reactivity may occur). Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites and is generally not a naturally occurring antibody. The term “multispecific binding protein” refers to a binding protein that can bind two or more related or unrelated targets, including a binding protein capable of binding two or more different epitopes of the same target molecule.

“Negative predictive value” or “NPV” as used interchangeably herein refers to the probability that a subject has a negative outcome given that they have a negative test result.

“Non-point-of-care device” refers to a device that is not a point-of-care device or a single use device. A non-point-of-care device refers to any device that does not meet any of the above limitations of a point-of-care or a single use device as defined herein. In some embodiments, the non-point-of-care device may be a relatively large instrument, such as a tabletop instrument. Accordingly, in some embodiments the non-point-of-care device is not a handheld instrument. In some embodiments, the non-point-of-care device is capable of performing an assay on more than one clinical sample simultaneously. Suitable non-point-of-care devices include, for example, the Architect or Alinity platforms produced by Abbott Core Laboratories.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, Ill.) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA). In some embodiments, the point-of-care device is a single-use device. The term “single-use device” or “single-use instrument” refers to a clinical diagnostic instrument that processes and performs a clinical diagnostic assay on a unit use basis (such as a single-use cartridge) for a single patient sample. A point-of-care instrument does not perform an assay on more than one clinical sample simultaneously. However, the point-of-care instrument may have the capability to measure more than one parameter (e.g., more than one analyte) in an individual clinical sample per unit use basis.

“Positive predictive value” or “PPV” as used interchangeably herein refers to the probability that a subject has a positive outcome given that they have a positive test result.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

A “receiver operating characteristic” curve or “ROC” curve refers to a graphical plot that illustrates the performance of a binary classifier system as its discrimination threshold is varied. For example, a ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” “biological sample,” and “patient sample” as used interchangeably herein may be a sample of blood, such as whole blood (including for example, capillary blood, venous blood, dried blood spot, etc.), serum or plasma, or tissue, saliva, urine, amniotic fluid, an oropharyngeal specimen, a nasopharyngeal specimens, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Additionally, the sample can be a nasopharyngeal or oropharyngeal sample obtained using one or more swabs that, once obtained, is placed in a sterile tube containing a virus transport media (VTM) or universal transport media (UTM), and retained therein or transferred to another media for testing.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, frozen sections taken for histologic purposes, blood (such as whole blood, dried blood spots, etc.), plasma, serum, saliva, red blood cells, platelets, interstitial fluid, cerebral spinal fluid, etc. Cell types and tissues may also include lymph fluid, cerebrospinal fluid, or any fluid collected by aspiration. A tissue or cell type may be provided by removing a sample of cells from a human and a non-human animal but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some embodiments, the sample is a blood sample (e.g., a whole blood sample, a serum sample, or a plasma sample). In some embodiments, the sample is a whole blood sample. In some embodiments, the sample is a capillary blood sample. In some embodiments, the sample is a dried blood spot. In some embodiments, the sample is a serum sample. In yet other embodiments, the sample is a plasma sample. In some embodiments, the sample is an oropharyngeal specimen. In other embodiments, the sample is a nasopharyngeal specimen. In other embodiments, the sample is sputum. In other embodiments, the sample is endotracheal aspirate. In still yet other embodiments, the sample is bronchoalveolar lavage. In still yet other embodiments, the sample is a saliva sample.

“Sensitivity” of an assay as used herein refers to the proportion of subjects for whom the outcome is positive that are correctly identified as positive (e.g., correctly identifying those subjects with a disease or medical condition for which they are being tested). For example, this might include correctly identifying subjects as having a TBI as distinct from those who do not have a TBI, correctly identifying subjects having a moderate, severe, or moderate-to-severe TBI as distinct from those having a mild TBI, correctly identifying subjects as having a mild TBI as distinct from those having a moderate, severe, or moderate-to-severe TBI, correctly identifying subjects as having a moderate, severe, or moderate-to-severe TBI as distinct from those having no TBI or correctly identifying subjects as having a mild TBI as distinct from those having no TBI etc. . . .

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects not having an TBI as distinct from those who do have a TBI, correctly identifying subjects not having a moderate, severe, or moderate-to-severe TBI as distinct from those having a mild TBI, correctly identifying subjects as not having a mild TBI as distinct from those having a moderate, severe, or moderate-to-severe TBI, etc.).

“Series of calibrating compositions” refers to a plurality of compositions comprising a known concentration of UCH-L1, wherein each of the compositions differs from the other compositions in the series by the concentration of UCH-L11; and/or (2) GFAP, wherein each composition differs from the other compositions in the series by the concentration of GFAP.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Subacute” as used herein refers to an injury (e.g., such as a traumatic brain injury) that is between an acute and chronic stage. Subacute refers to an injury from after about 24 hours up to about one, two or three weeks after an actual or suspected injury to the head. The term “acute” as used herein refers to an injury from time zero (inception or occurrence of an actual or suspected injury to the head) up until about 24 hours after the actual or suspected injury to the head.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In some embodiments, the subject is a human. The subject or patient may be undergoing other forms of treatment.

“Treat,” “treating” or “treatment” are each used interchangeably herein to describe reversing, alleviating, or inhibiting the progress of a disease and/or injury, or one or more symptoms of such disease, to which such term applies. In some aspects, a treatment may be either performed in an acute or chronic way. Depending on the condition of the subject, the term also refers to preventing a disease or injury, and includes preventing the onset of a disease or injury, or preventing the symptoms associated with a disease or injury. “preventing” also refers to preventing the recurrence of a disease or injury or of one or more symptoms associated with such disease or injury. “treatment” and “therapeutically,” refer to the act of treating, as “treating” is defined above. In some aspects, the prevention or treatment of a disease can be done prior to affliction or injury, such as, for example, to reduce the severity of a disease or injury or symptoms associated with a disease or an injury. Such prevention or reduction can include (a) administration of one or more pharmaceutical composition and/or one or more nutritional compositions to a subject; (b) the use of one or more of physical therapy, occupational therapy, and/or counseling; or (c) any combinations of (a) and (b).

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” “moderate-to-severe”, or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more fires, chemicals or toxins (such as molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, a viral infection (e.g., SARS-COV-2, meningitis, etc.), fungal infection (e.g., meningitis), bacterial infection (e.g., meningitis), or any combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a head injury where a subject may or may not experience a loss of consciousness. For subjects that experience a loss of consciousness, it is typically brief, usually lasting only a few seconds or minutes. Mild TBI is also referred to as a concussion, minor head trauma, minor TBI, minor brain injury, and minor head injury. While MRI and CT scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma Scale score of between 13-15 (such as 13-15 or 14-15). Fifteen percent (15%) of people with mild TBI have symptoms that last 3 months or more. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma Scale score of between 9-13 (such as 9-12 or 9-13). The individual with moderate TBI may have abnormal brain imaging results. “Severe TBI” as used herein refers to a brain injury where loss of consciousness is more than 24 hours and memory loss after the injury or penetrating skull injury longer than 24 hours and the subject has a Glasgow Coma Scale score between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

“Moderate-to-severe” TBI as used herein refers to a spectrum of brain injury that includes a change from moderate-to-severe TBI over time and thus encompasses (e.g., temporally) moderate TBI alone, severe TBI alone, and moderate-to-severe TBI combined. For example, in some clinical situations, a subject may initially be diagnosed as having a moderate TBI but who, over the course of time (minutes, hours or days), progresses to having a severe TBI (such, as for example, in situations when there is a brain bleed). Alternatively, in some clinical situations, a subject may initially be diagnosed as having a severe TBI but who, over the course of time (minutes, hours or days), progresses to having a moderate TBI. Such subjects would be examples of patients that could be classified as “moderate-to-severe”. Common symptoms of moderate-to-severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing issues, such as decrease or loss of hearing, ringing in the cars (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, problems with control of bowel and bladder, sleep disorders, loss of stamina, appetite changes, problems with regulation of body temperature, menstrual difficulties, dependent behaviors, issues with emotional ability or stability, lack of motivation, irritability, aggression, depression, disinhibition, or denial/lack of awareness. Subjects having a moderate-to-severe TBI can have a Glasgow Coma scale score from 3-12 (which includes the range of 9-12 for a moderate TBI, and 3-8 for a severe TBI).

“Ubiquitin carboxy-terminal hydrolase L1” or “UCH-L1” as used interchangeably herein refers to a deubiquitinating enzyme encoded by the UCH-L1 gene in humans and by UCH-L1 gene counterparts in other species. UCH-L1, also known as ubiquitin carboxyl-terminal esterase L1 and ubiquitin thioesterase, is a member of a gene family whose products hydrolyze small C-terminal adducts of ubiquitin to generate the ubiquitin monomer.

“UCH-L1 status” can mean either the level or amount of UCH-L1 at a point in time (such as with a single measure of UCH-L1), the level or amount of UCH-L1 associated with monitoring (such as with a repeat test on a subject to identify an increase or decrease in UCH-L1 amount), the level or amount of UCH-L1 associated with treatment for traumatic brain injury (whether a primary brain injury and/or a secondary brain injury) or combinations thereof.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-UCH-L1 antibody that differs from the corresponding fragment of anti-UCH-L1 antibody in amino acid sequence but is still antigenically reactive and can compete with the corresponding fragment of anti-UCH-L1 antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Aiding in the Diagnosis and Evaluation of a Subject that has Sustained or May have Sustained an Acquired Brain Injury (ABI)

The present disclosure relates, among other methods, to a method of aiding in determining whether a subject, such as a human subject, who has sustained, may have sustained, or is suspected of sustaining an injury to the head has more likely than not, sustained an acquired brain injury (ABI), such as a traumatic brain injury (TBI). In some aspects, the method can involve the steps of: (a) obtaining or receiving a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI; and (b) determining that the subject:
a. has not suffered or recovered from an actual ABI (such as, for example, a mild TBI, a moderate TBI, a severe TBI, or a moderate-to-severe TBI) if the subject's: (a) GFAP level is less than about X pg/mL; (b) UCH-L1 level is less than about Y pg/mL; or (c) GFAP level is less than about X pg/mL and UCH-L1 level is less than about Y pg/mL;
b. should be monitored for recovery from an actual ABI (such as, for example, a mild TBI, a moderate TBI, a severe TBI, or a moderate-to-severe TBI) if the subject's: (a) GFAP level is between a range of about X pg/mL to about X1 pg/mL; (b) UCH-L1 level is between a range of about Y pg/mL to about Y1 pg/mL; or (c) GFAP level is between a range of about X pg/mL to about X1 pg/mL and UCH-L1 level is between a range of about Y pg/mL to about Y1 pg/mL; and/or
c. sustained and is not recovering from an actual ABI (such as, for example, a mild TBI, a moderate TBI, a severe TBI, or a moderate-to-severe TBI) if the subject's: (a) GFAP level is greater than about X1 pg/mL; (b) UCH-L1 level is greater than about Y1 pg/mL; or (c) GFAP level is greater than about X1 pg/mL and UCH-L1 level is greater than about Y1 pg/mL,
where X is 16, 17, or 18; X1 is 291, 294, 307, 375, 415, 425, or 431; Y is 49, 51, 53, 55, 56, 61 or 63; Y1 is 243, 248, 250, 267, or 275. In some aspects, X is 16 and X1 is 291, 294, 307, 375, 415, 425, or 421. In other aspects, X is 17 and X1 is 291, 294, 307, 375, 415, 425, or 421. In still further aspects, X is 17 and X1 is 291, 294, 307, 375, 415, 425, or 421. In yet other aspects, Y is 49 and Y1 is 291, 294, 307, 375, 415, 425, or 421. In still other aspects, Y is 51 and Y1 is 291, 294, 307, 375, 415, 425, or 421. In yet other aspects, Y is 53 and Y1 is 291, 294, 307, 375, 415, 425, or 421. In still other aspects, Y is 55 and Y1 is 291, 294, 307, 375, 415, 425, or 421. In still yet other aspects, Y is 56 and Y1 is 291, 294, 307, 375, 415, 425, or 421. In yet further aspects, Y is 61 and Y1 is 291, 294, 307, 375, 415, 425, or 421.

In further aspects, the method can involve the steps of: (a) performing, simultaneously or sequentially (in any order), one or more assays on at least one or more samples obtained from the subject from within after about twenty-four hours to (i) about one week, (ii) about two weeks, or (iii) about three weeks, after an actual or suspected ABI to measure or detect a level of a biomarker in the sample, where the biomarker is glial fibrillary acidic protein (GFAP), ubiquitin carboxy-terminal hydrolase L1 (UCH-L1), or a combination of GFAP and UCH-L1; and (b) determining that the subject:
a. has not suffered or recovered from an actual ABI (such as, for example, a mild TBI, a moderate TBI, a severe TBI, or a moderate-to-severe TBI) if the subject's: (a) GFAP level is less than about X pg/mL; (b) UCH-L1 level is less than about Y pg/mL; or (c) GFAP level is less than about X pg/mL and UCH-L1 level is less than about Y pg/mL;
b. should be monitored for recovery from an actual ABI (such as, for example, a mild TBI, a moderate TBI, a severe TBI, or a moderate-to-severe TBI) if the subject's: (a) GFAP level is between a range of about X pg/mL to about X1 pg/mL; (b) UCH-L1 level is between a range of about Y pg/mL to about Y1 pg/mL; or (c) GFAP level is between a range of about X pg/mL to about X1 pg/mL and UCH-L1 level is between a range of about Y pg/mL to about Y1 pg/mL; and/or
c. sustained and is not recovering from an actual ABI (such as, for example, a mild TBI, a moderate TBI, a severe TBI, or a moderate-to-severe TBI) if the subject's: (a) GFAP level is greater than about X1 pg/mL; (b) UCH-L1 level is greater than about Y1 pg/mL; or (c) GFAP level is greater than about X1 pg/mL and UCH-L1 level is greater than about Y1 pg/mL,
where X is 16, 17, or 18; X1 is 291, 294, 307, 375, 415, 425, or 431; Y is 49, 51, 53, 55, 56, 61 or 63; Y1 is 243, 248, 250, 267, or 275. In some aspects, X is 16 and X1 is 291, 294, 307, 375, 415, 425, or 421. In other aspects, X is 17 and X1 is 291, 294, 307, 375, 415, 425, or 421. In still further aspects, X is 17 and X1 is 291, 294, 307, 375, 415, 425, or 421. In yet other aspects, Y is 49 and Y1 is 291, 294, 307, 375, 415, 425, or 421. In still other aspects, Y is 51 and Y1 is 291, 294, 307, 375, 415, 425, or 421. In yet other aspects, Y is 53 and Y1 is 291, 294, 307, 375, 415, 425, or 421. In still other aspects, Y is 55 and Y1 is 291, 294, 307, 375, 415, 425, or 421. In still yet other aspects, Y is 56 and Y1 is 291, 294, 307, 375, 415, 425, or 421. In yet further aspects, Y is 61 and Y1 is 291, 294, 307, 375, 415, 425, or 421.

In one aspect, the methods described herein involve determining that a subject has not suffered an ABI or has recovered from an actual ABI (such as, for example, a mild TBI, a moderate TBI, a severe TBI, or a moderate-to-severe TBI) when the subject's: (a) GFAP level is less than about 16, about 17, or about 18 pg/mL; (b) UCH-L1 level is less than about 49, about 51, about 53, about 55, about 56, about 61 or about 63 pg/mL; or (c) GFAP level is less than about 16, about 17, or about 18 pg/mL and UCH-L1 level is less than about 49, about 51, about 53, about 55, about 56, about 61 or about 63 pg/mL.

In another aspect, the methods described herein involve determining that a subject who has suffered an ABI (such as, for example, a mild TBI, a moderate TBI, a severe TBI, or a moderate-to-severe TBI) or is suspected of having suffered an ABI (such as, for example, a mild TBI, a moderate TBI, a severe TBI, or a moderate-to-severe TBI), be monitored for recovery from an actual ABI (such as, for example, a mild TBI, a moderate TBI, a severe TBI, or a moderate-to-severe TBI) when the subject's: (a) GFAP level is between a range of about 16 pg/mL to about 291 pg/mL, about 16 pg/mL to about 294 pg/mL, about 16 pg/mL to about 307 pg/mL, about 16 pg/mL to about 375 pg/mL, about 16 pg/mL to about 415 pg/mL, about 16 pg/mL to about 425 pg/mL, about 16 pg/mL to about 431 pg/mL, 17 pg/mL to about 291 pg/mL, about 17 pg/mL to about 294 pg/mL, about 17 pg/mL to about 307 pg/mL, about 17 pg/mL to about 375 pg/mL, about 17 pg/mL to about 415 pg/mL, about 17 pg/mL to about 425 pg/mL, about 17 pg/mL to about 431 pg/mL; 18 pg/mL to about 291 pg/mL, about 18 pg/mL to about 294 pg/mL, about 18 pg/mL to about 307 pg/mL, about 18 pg/mL to about 375 pg/mL, about 18 pg/mL to about 415 pg/mL, about 18 pg/mL to about 425 pg/mL, or about 18 pg/mL to about 431 pg/mL; (b) UCH-L1 level is between a range of about 49 pg/mL to about 243 pg/mL, about 49 pg/mL to about 248 pg/mL, about 49 pg/mL to about 250 pg/mL, about 49 pg/mL to about 267 pg/mL, about 49 pg/mL to about 275 pg/mL, 51 pg/mL to about 243 pg/mL, about 51 pg/mL to about 248 pg/mL, about 51 pg/mL to about 250 pg/mL, about 51 pg/mL to about 267 pg/mL, about 51 pg/mL to about 275 pg/mL, 53 pg/mL to about 243 pg/mL, about 53 pg/mL to about 248 pg/mL, about 53 pg/mL to about 250 pg/mL, about 53 pg/mL to about 267 pg/mL, about 53 pg/mL to about 275 pg/mL, 55 pg/mL to about 243 pg/mL, about 55 pg/mL to about 248 pg/mL, about 55 pg/mL to about 250 pg/mL, about 55 pg/mL to about 267 pg/mL, about 55 pg/mL to about 275 pg/mL, 56 pg/mL to about 243 pg/mL, about 56 pg/mL to about 248 pg/mL, about 56 pg/mL to about 250 pg/mL, about 56 pg/mL to about 267 pg/mL, about 56 pg/mL to about 275 pg/mL, 61 pg/mL to about 243 pg/mL, about 61 pg/mL to about 248 pg/mL, about 61 pg/mL to about 250 pg/mL, about 61 pg/mL to about 267 pg/mL, about 61 pg/mL to about 275 pg/mL, 63 pg/mL to about 243 pg/mL, about 63 pg/mL to about 248 pg/mL, about 63 pg/mL to about 250 pg/mL, about 63 pg/mL to about 267 pg/mL, or about 63 pg/mL to about 275 pg/mL; or (c) GFAP level is between a range of about 16 pg/mL to about 291 pg/mL, about 16 pg/mL to about 294 pg/mL, about 16 pg/mL to about 307 pg/mL, about 16 pg/mL to about 375 pg/mL, about 16 pg/mL to about 415 pg/mL, about 16 pg/mL to about 425 pg/mL, about 16 pg/mL to about 431 pg/mL, 17 pg/mL to about 291 pg/mL, about 17 pg/mL to about 294 pg/mL, about 17 pg/mL to about 307 pg/mL, about 17 pg/mL to about 375 pg/mL, about 17 pg/mL to about 415 pg/mL, about 17 pg/mL to about 425 pg/mL, about 17 pg/mL to about 431 pg/mL; 18 pg/mL to about 291 pg/mL, about 18 pg/mL to about 294 pg/mL, about 18 pg/mL to about 307 pg/mL, about 18 pg/mL to about 375 pg/mL, about 18 pg/mL to about 415 pg/mL, about 18 pg/mL to about 425 pg/mL, or about 18 pg/mL to about 431 pg/mL and UCH-L1 level is between a range of about 49 pg/mL to about 243 pg/mL, about 49 pg/mL to about 248 pg/mL, about 49 pg/mL to about 250 pg/mL, about 49 pg/mL to about 267 pg/mL, about 49 pg/mL to about 275 pg/mL, 51 pg/mL to about 243 pg/mL, about 51 pg/mL to about 248 pg/mL, about 51 pg/mL to about 250 pg/mL, about 51 pg/mL to about 267 pg/mL, about 51 pg/mL to about 275 pg/mL, 53 pg/mL to about 243 pg/mL, about 53 pg/mL to about 248 pg/mL, about 53 pg/mL to about 250 pg/mL, about 53 pg/mL to about 267 pg/mL, about 53 pg/mL to about 275 pg/mL, 55 pg/mL to about 243 pg/mL, about 55 pg/mL to about 248 pg/mL, about 55 pg/mL to about 250 pg/mL, about 55 pg/mL to about 267 pg/mL, about 55 pg/mL to about 275 pg/mL, 56 pg/mL to about 243 pg/mL, about 56 pg/mL to about 248 pg/mL, about 56 pg/mL to about 250 pg/mL, about 56 pg/mL to about 267 pg/mL, about 56 pg/mL to about 275 pg/mL, 61 pg/mL to about 243 pg/mL, about 61 pg/mL to about 248 pg/mL, about 61 pg/mL to about 250 pg/mL, about 61 pg/mL to about 267 pg/mL, about 61 pg/mL to about 275 pg/mL, 63 pg/mL to about 243 pg/mL, about 63 pg/mL to about 248 pg/mL, about 63 pg/mL to about 250 pg/mL, about 63 pg/mL to about 267 pg/mL, or about 63 pg/mL to about 275 pg/mL.

In still yet a further aspect, the methods described herein involve determining whether a subject has sustained an ABI, such as a TBI (e.g., a mild TBI, a moderate TBI, a severe TBI or a moderate-to-severe TBI), and whether the subject is not recovering from the actual ABI when the subject's: (a) GFAP level is greater than about 291 pg/mL, about 294 pg/mL, about 307 pg/mL, about 375 pg/mL, about 415 pg/mL, about 425 pg/mL, or about 431 pg/mL; (b) UCH-L1 level is greater than about 243 pg/mL, about 248 pg/mL, about 250 pg/mL, about 267 pg/mL or about 275 pg/mL; or (c) GFAP level is greater than about 291 pg/mL, about 294 pg/mL, about 307 pg/mL, about 375 pg/mL, about 415 pg/mL, about 425 pg/mL, or about 431 pg/mL and UCH-L1 level is greater than about 243 pg/mL, about 248 pg/mL, about 250 pg/mL, about 267 pg/mL or about 275 pg/mL. For example, the methods described herein can be used to determine whether a subject has sustained an ABI, such as a TBI, is not recovering from the actual ABI. In these instances where a subject is not recovering from an actual ABI, different treatment and/or monitoring options can be pursued depending on the judgment of a clinician.

In some embodiments, the sample used in the method can be a biological sample. In some aspects, the method described herein can include obtaining at least one sample from a subject (e.g., such as a human subject), at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject and contacting the sample with an antibody for a biomarker of ABI, such as ubiquitin carboxy-terminal hydrolase L1 (UCH-L1), glial fibrillary acidic protein (GFAP), or a combination thereof, to allow formation of a complex of the antibody and the biomarker. The method also includes detecting the resulting antibody-biomarker complex.

In some embodiments, one or more samples is taken from the subject, such as a human subject, at one or multiple timepoints in after about 24 hours after an ABI (e.g., an actual injury) or suspected ABI, after about 36 hours after an ABI or suspected ABI, after about 40 hours after an ABI or suspected ABI, after about 48 hours after an ABI or suspected ABI, after about 3 days after an ABI or suspected ABI, after about 4 days after an ABI or suspected ABI, after about 4 days after an ABI or suspected ABI, after about 5 days after an ABI or suspected ABI, after about 6 days after an ABI or suspected ABI, after about 1 week after an ABI or suspected ABI, within about 25 hours to about 3 weeks after an ABI or suspected ABI, within about 25 hours to about two weeks after an ABI or suspected ABI, within about 25 hours to about 1 week after an ABI or suspected ABI, within about 1 week to about 2 weeks after ABI or suspected ABI, about 1 week to about 2 weeks after an ABI or suspected ABI, about 2 weeks to about 3 weeks after ABI or suspected ABI, or about 3 weeks after ABI or suspected ABI. In some aspects, the ABI is a TBI.

Generally, a reference level or level of the biomarker, such as UCH-L1, GFAP, or a combination thereof, can be employed as a benchmark against which to assess results obtained upon assaying a test sample for UCH-L1, GFAP or UCH-L1 and GFAP. Generally, in making such a comparison, the reference level or level of the biomarker, such as UCH-L1, GFAP, or a combination thereof, is obtained by running a particular assay a sufficient number of times and under appropriate conditions such that a linkage or association of analyte presence, amount or concentration with a particular stage or endpoint of ABI (e.g., such as a TBI) or with particular indicia can be made. Typically, the reference level or level of the biomarker, such as UCH-L1, GFAP, or a combination thereof, is obtained with assays of reference subjects (or populations of subjects). The biomarker, such as UCH-L1, GFAP, or a combination thereof, measured can include fragments thereof, degradation products thereof, and/or enzymatic cleavage products thereof.

In some aspects, the GFAP, UCH-L1, or GFAP and UCH-L1 reference levels or levels used in the methods described herein are those provided below in Tables 2 and 3. These reference levels can be used when determining the concentration of GFAP alone, UCH-L1 alone, or a combination of GFAP and UCH-L1.

TABLE 2

| Option | Determining that a subject: | GFAP level (pg/mL) |
|---|---|---|
| 1 | Has not suffered or recovered from an actual ABI | Less than about 16 |
| | Should be monitored for recovery from an actual ABI | Between a range of about 16 |
| | | to about 291 |
| | Sustained and is not recovering from an actual ABI | Greater than about 291 |
| 2 | Has not suffered or recovered from an actual ABI | Less than about 16 |
| | Should be monitored for recovery from an actual ABI | Between a range of about 16 |
| | | to about 294 |
| | Sustained and is not recovering from an actual ABI | Greater than about 294 |
| 3 | Has not suffered or recovered from an actual ABI | Less than about 16 |
| | Should be monitored for recovery from an actual ABI | Between a range of about 16 |
| | | to about 307 |
| | Sustained and is not recovering from an actual ABI | Greater than about 307 |
| 4 | Has not suffered or recovered from an actual ABI | Less than about 16 |
| | Should be monitored for recovery from an actual ABI | Between a range of about 16 |
| | | to about 375 |
| | Sustained and is not recovering from an actual ABI | Greater than about 375 |
| 5 | Has not suffered or recovered from an actual ABI | Less than about 16 |
| | Should be monitored for recovery from an actual ABI | Between a range of about 16 |
| | | to about 415 |
| | Sustained and is not recovering from an actual ABI | Greater than about 415 |
| 6 | Has not suffered or recovered from an actual ABI | Less than about 16 |
| | Should be monitored for recovery from an actual ABI | Between a range of about 16 |
| | | to about 425 |
| | Sustained and is not recovering from an actual ABI | Greater than about 425 |
| 7 | Has not suffered or recovered from an actual ABI | Less than about 17 |
| | Should be monitored for recovery from an actual ABI | Between a range of about 17 |
| | | to about 291 |
| | Sustained and is not recovering from an actual ABI | Greater than about 291 |
| 8 | Has not suffered or recovered from an actual ABI | Less than about 17 |
| | Should be monitored for recovery from an actual ABI | Between a range of about 17 |
| | | to about 294 |
| | Sustained and is not recovering from an actual ABI | Greater than about 294 |
| 9 | Has not suffered or recovered from an actual ABI | Less than about 17 |
| | Should be monitored for recovery from an actual ABI | Between a range of about 17 |
| | | to about 307 |
| | Sustained and is not recovering from an actual ABI | Greater than about 307 |
| 10 | Has not suffered or recovered from an actual ABI | Less than about 17 |
| | Should be monitored for recovery from an actual ABI | Between a range of about 17 |
| | | to about 375 |
| | Sustained and is not recovering from an actual ABI | Greater than about 375 |
| 11 | Has not suffered or recovered from an actual ABI | Less than about 17 |
| | Should be monitored for recovery from an actual ABI | Between a range of about 17 |
| | | to about 415 |
| | Sustained and is not recovering from an actual ABI | Greater than about 415 |
| 12 | Has not suffered or recovered from an actual ABI | Less than about 17 |
| | Should be monitored for recovery from an actual ABI | Between a range of about 17 |
| | | to about 425 |
| | Sustained and is not recovering from an actual ABI | Greater than about 425 |
| 13 | Has not suffered or recovered from an actual ABI | Less than about 18 |
| | Should be monitored for recovery from an actual ABI | Between a range of about 18 |
| | | to about 291 |
| | Sustained and is not recovering from an actual ABI | Greater than about 291 |
| 14 | Has not suffered or recovered from an actual ABI | Less than about 18 |
| | Should be monitored for recovery from an actual ABI | Between a range of about 18 |
| | | to about 294 |
| | Sustained and is not recovering from an actual ABI | Greater than about 294 |
| 15 | Has not suffered or recovered from an actual ABI | Less than about 18 |
| | Should be monitored for recovery from an actual ABI | Between a range of about 18 |
| | | to about 307 |
| | Sustained and is not recovering from an actual ABI | Greater than about 307 |
| 16 | Has not suffered or recovered from an actual ABI | Less than about 18 |
| | Should be monitored for recovery from an actual ABI | Between a range of about 18 |
| | | to about 375 |
| | Sustained and is not recovering from an actual ABI | Greater than about 375 |
| 17 | Has not suffered or recovered from an actual ABI | Less than about 18 |
| | Should be monitored for recovery from an actual ABI | Between a range of about 18 |
| | | to about 415 |
| | Sustained and is not recovering from an actual ABI | Greater than about 415 |
| 18 | Has not suffered or recovered from an actual ABI | Less than about 18 |
| | Should be monitored for recovery from an actual ABI | Between a range of about 18 |
| | | to about 425 |
| | Sustained and is not recovering from an actual ABI | Greater than about 425 |

TABLE 3

| | | UCH-L1 level |
|---|---|---|
| Option | Determining that a subject: | (pg/mL) |
| 1 | Has not suffered or recovered from an actual ABI | Less than about 49 |
| | Should be monitored for recovery from an actual ABI | Between a range of about 49 to |
| | | about 243 |
| | Sustained and is not recovering from an actual ABI | Greater than about 243 |
| 2 | Has not suffered or recovered from an actual ABI | Less than about 49 |
| | Should be monitored for recovery from an actual ABI | Between a range of about 49 to |
| | | about 248 |
| | Sustained and is not recovering from an actual ABI | Greater than about 248 |
| 3 | Has not suffered or recovered from an actual ABI | Less than about 49 |
| | Should be monitored for recovery from an actual ABI | Between a range of about 49 to |
| | | about 250 |
| | Sustained and is not recovering from an actual ABI | Greater than about 250 |
| 4 | Has not suffered or recovered from an actual ABI | Less than about 49 |
| | Should be monitored for recovery from an actual ABI | Between a range of about 49 to |
| | | about 267 |
| | Sustained and is not recovering from an actual ABI | Greater than about 267 |
| 5 | Has not suffered or recovered from an actual ABI | Less than about 49 |
| | Should be monitored for recovery from an actual ABI | Between a range of about 49 to |
| | | about 275 |
| | Sustained and is not recovering from an actual ABI | Greater than about 275 |
| 6 | Has not suffered or recovered from an actual ABI | Less than about 51 |
| | Should be monitored for recovery from an actual ABI | Between a range of about 51 to |
| | | about 243 |
| | Sustained and is not recovering from an actual ABI | Greater than about 243 |
| 7 | Has not suffered or recovered from an actual ABI | Less than about 51 |
| | Should be monitored for recovery from an actual ABI | Between a range of about 51 to |
| | | about 248 |
| | Sustained and is not recovering from an actual ABI | Greater than about 248 |
| 8 | Has not suffered or recovered from an actual ABI | Less than about 51 |
| | Should be monitored for recovery from an actual ABI | Between a range of about 49 to |
| | | about 250 |
| | Sustained and is not recovering from an actual ABI | Greater than about 250 |
| 9 | Has not suffered or recovered from an actual ABI | Less than about 51 |
| | Should be monitored for recovery from an actual ABI | Between a range of about 51 to |
| | | about 267 |
| | Sustained and is not recovering from an actual ABI | Greater than about 267 |
| 10 | Has not suffered or recovered from an actual ABI | Less than about 51 |
| | Should be monitored for recovery from an actual ABI | Between a range of about 51 to |
| | | about 275 |
| | Sustained and is not recovering from an actual ABI | Greater than about 275 |
| 11 | Has not suffered or recovered from an actual ABI | Less than about 53 |
| | Should be monitored for recovery from an actual ABI | Between a range of about 53 to |
| | | about 243 |
| | Sustained and is not recovering from an actual ABI | Greater than about 243 |
| 12 | Has not suffered or recovered from an actual ABI | Less than about 53 |
| | Should be monitored for recovery from an actual ABI | Between a range of about 53 to |
| | | about 248 |
| | Sustained and is not recovering from an actual ABI | Greater than about 248 |
| 13 | Has not suffered or recovered from an actual ABI | Less than about 53 |
| | Should be monitored for recovery from an actual ABI | Between a range of about 53 to |
| | | about 250 |
| | Sustained and is not recovering from an actual ABI | Greater than about 250 |
| 14 | Has not suffered or recovered from an actual ABI | Less than about 53 |
| | Should be monitored for recovery from an actual ABI | Between a range of about 53 to |
| | | about 267 |
| | Sustained and is not recovering from an actual ABI | Greater than about 267 |
| 15 | Has not suffered or recovered from an actual ABI | Less than about 53 |
| | Should be monitored for recovery from an actual ABI | Between a range of about 53 to |
| | | about 275 |
| | Sustained and is not recovering from an actual ABI | Greater than about 275 |
| 16 | Has not suffered or recovered from an actual ABI | Less than about 55 |
| | Should be monitored for recovery from an actual ABI | Between a range of about 55 to |
| | | about 243 |
| | Sustained and is not recovering from an actual ABI | Greater than about 243 |
| 17 | Has not suffered or recovered from an actual ABI | Less than about 55 |
| | Should be monitored for recovery from an actual ABI | Between a range of about 55 to |
| | | about 248 |
| | Sustained and is not recovering from an actual ABI | Greater than about 248 |
| 18 | Has not suffered or recovered from an actual ABI | Less than about 55 |
| | Should be monitored for recovery from an actual ABI | Between a range of about 55 to |
| | | about 250 |
| | Sustained and is not recovering from an actual ABI | Greater than about 250 |
| 19 | Has not suffered or recovered from an actual ABI | Less than about 55 |
| | Should be monitored for recovery from an actual ABI | Between a range of about 55 to |
| | | about 267 |
| | Sustained and is not recovering from an actual ABI | Greater than about 267 |
| 20 | Has not suffered or recovered from an actual ABI | Less than about 55 |
| | Should be monitored for recovery from an actual ABI | Between a range of about 55 to |
| | | about 275 |
| | Sustained and is not recovering from an actual ABI | Greater than about 275 |
| 21 | Has not suffered or recovered from an actual ABI | Less than about 56 |
| | Should be monitored for recovery from an actual ABI | Between a range of about 56 to |
| | | about 243 |
| | Sustained and is not recovering from an actual ABI | Greater than about 243 |
| 22 | Has not suffered or recovered from an actual ABI | Less than about 56 |
| | Should be monitored for recovery from an actual ABI | Between a range of about 56 to |
| | | about 248 |
| | Sustained and is not recovering from an actual ABI | Greater than about 248 |
| 23 | Has not suffered or recovered from an actual ABI | Less than about 56 |
| | Should be monitored for recovery from an actual ABI | Between a range of about 56 to |
| | | 250 |
| | Sustained and is not recovering from an actual ABI | Greater than about 250 |
| 24 | Has not suffered or recovered from an actual ABI | Less than about 56 |
| | Should be monitored for recovery from an actual ABI | Between a range of about 56 to |
| | | about 267 |
| | Sustained and is not recovering from an actual ABI | Greater than about 267 |
| 25 | Has not suffered or recovered from an actual ABI | Less than about 56 |
| | Should be monitored for recovery from an actual ABI | Between a range of about 56 to |
| | | about 275 |
| | Sustained and is not recovering from an actual ABI | Greater than about 275 |
| 26 | Has not suffered or recovered from an actual ABI | Less than about 61 |
| | Should be monitored for recovery from an actual ABI | Between a range of about 61 to |
| | | about 243 |
| | Sustained and is not recovering from an actual ABI | Greater than about 243 |
| 27 | Has not suffered or recovered from an actual ABI | Less than about 61 |
| | Should be monitored for recovery from an actual ABI | Between a range of about 61 to |
| | | about 248 |
| | Sustained and is not recovering from an actual ABI | Greater than about 248 |
| 28 | Has not suffered or recovered from an actual ABI | Less than about 61 |
| | Should be monitored for recovery from an actual ABI | Between a range of about 61 to |
| | | about 250 |
| | Sustained and is not recovering from an actual ABI | Greater than about 250 |
| 29 | Has not suffered or recovered from an actual ABI | Less than about 61 |
| | Should be monitored for recovery from an actual ABI | Between a range of about 61 to |
| | | about 267 |
| | Sustained and is not recovering from an actual ABI | Greater than about 267 |
| 30 | Has not suffered or recovered from an actual ABI | Less than about 61 |
| | Should be monitored for recovery from an actual ABI | Between a range of about 61 to |
| | | about 275 |
| | Sustained and is not recovering from an actual ABI | Greater than about 275 |
| 31 | Has not suffered or recovered from an actual ABI | Less than about 63 |
| | Should be monitored for recovery from an actual ABI | Between a range of about 63 to |
| | | about 243 |
| | Sustained and is not recovering from an actual ABI | Greater than about 243 |
| 32 | Has not suffered or recovered from an actual ABI | Less than about 63 |
| | Should be monitored for recovery from an actual ABI | Between a range of about 63 to |
| | | about 248 |
| | Sustained and is not recovering from an actual ABI | Greater than about 248 |
| 33 | Has not suffered or recovered from an actual ABI | Less than about 63 |
| | Should be monitored for recovery from an actual ABI | Between a range of about 63 to |
| | | about 250 |
| | Sustained and is not recovering from an actual ABI | Greater than about 250 |
| 34 | Has not suffered or recovered from an actual ABI | Less than about 63 |
| | Should be monitored for recovery from an actual ABI | Between a range of about 63 to |
| | | about 267 |
| | Sustained and is not recovering from an actual ABI | Greater than about 267 |
| 35 | Has not suffered or recovered from an actual ABI | Less than about 63 |
| | Should be monitored for recovery from an actual ABI | Between a range of about 63 to |
| | | about 275 |
| | Sustained and is not recovering from an actual ABI | Greater than about 275 |

In still other aspects, the reference level or level for GFAP. UCH-L1, or GFAP or UCH-L1 is correlated with one or more measures, such as, for example, a subject's Glasgow Coma Scale Score, a subject's Extended Glasgow Outcome Scale Score, a positive CT scan for a TBI, a negative CT scan for a TBI, or any combination thereof. In some aspects, the reference level or level for GFAP, UCH-L1 or GFAP, or UCH-L1 is correlated with a subject's Glasgow Coma Scale Score and the type of ABI. In some aspects, the correlation between the reference level or level and Glasgow Coma Scale score will depend on whether a subject has suffered a mild TBI, a moderate TBI, a severe TBI, or moderate-to-severe TBI. In other aspects, the reference level or level for GFAP, UCH-L1, or GFAP and UCH-L1 is correlated with a subject's Extended Glasgow Outcome Scale Score. In some aspects, the correlation between the reference level or level and Extended Glasgow Outcome Scale Score will dependent on whether a subject has suffered a mild TBI, a moderate TBI, a severe TBI, or moderate-to-severe TBI. In some aspects, the reference level or level for GFAP, UCH-L1, or GFAP and UCH-L1 will correlate with a Extended Glasgow Outcome Scale score of 0-3 or 4-8. In still other aspects, the reference level or level for GFAP, UCH-L1, or GFAP and UCH-L1 is correlated with a positive head CT scan and/or a negative head CT scan.

In still other aspects, the reference level or level for GFAP, UCH-L1, or GFAP or UCH-L1 is correlated with one or more measures as shown below in Tables 4-6. These reference levels can be used when determining the concentration of GFAP alone, UCH-L1 alone, or a combination of GFAP and UCH-L1.

TABLE 4

| Option | Determining that a subject: | GFAP level (pg/mL) | Measure |
|---|---|---|---|
| 1 | Has not suffered or recovered | Less than about 17 | GCS |
| | from an actual ABI | | (such as a mild TBI; GCS 13- |
| | | | 15) |
| | Should be monitored for | Between a range of | GCS |
| | recovery from an actual ABI | about 17 to about 425 | (such as a moderate, severe, |
| | | | or moderate-to-severe TBI; |
| | | | GCS 3-12) |
| | Sustained and is not | Greater than about | GCS |
| | recovering from an actual ABI | 425 | (such as a moderate, severe, |
| | | | or moderate-to-severe TBI; |
| | | | GCS 3-12) |
| 2 | Has not suffered or recovered | Less than about 17 | GCS |
| | from an actual ABI | | (such as a mild TBI; GCS 13- |
| | | | 15) |
| | Should be monitored for | Between a range of | GCS |
| | recovery from an actual ABI | about 17 to about 431 | (such as a moderate, severe, |
| | | | or moderate-to-severe TBI; |
| | | | GCS 3-12) |
| | Sustained and is not | Greater than about | GCS |
| | recovering from an actual ABI | 431 | (such as a moderate, severe, |
| | | | or moderate-to-severe TBI; |
| | | | GCS 3-12) |
| 3 | Has not suffered or recovered | Less than about 17 | GCS |
| | from an actual ABI | | (such as a mild TBI; GCS 13- |
| | | | 15) |
| | Should be monitored for | Between a range of | GCS |
| | recovery from an actual ABI | about 17 to about 415 | (such as a moderate, severe, |
| | | | or moderate-to-severe TBI; |
| | | | GCS 3-12) |
| | Sustained and is not | Greater than about | GCS |
| | recovering from an actual ABI | 415 | (such as a moderate, severe, |
| | | | or moderate-to-severe TBI; |
| | | | GCS 3-12) |
| 4 | Has not suffered or recovered | Less than about 17 | GOSE |
| | from an actual ABI | | (such as a GOSE score of 4-8) |
| | Should be monitored for | Between a range of | GOSE |
| | recovery from an actual ABI | about 17 to about 431 | (such as a GOSE score of 0-3) |
| | Sustained and is not | Greater than about | GOSE |
| | recovering from an actual ABI | 431 | (such as a GOSE score of 0-3) |
| 5 | Has not suffered or recovered | Less than about 17 | GOSE |
| | from an actual ABI | | (such as a GOSE score of 4-8) |
| | Should be monitored for | Between a range of | GOSE |
| | recovery from an actual ABI | about 17 to about 375 | (such as a GOSE score of 0-3) |
| | Sustained and is not | Greater than about | GOSE |
| | recovering from an actual ABI | 375 | (such as a GOSE score of 0-3) |
| 6 | Has not suffered or recovered | Less than about 17 | CT |
| | from an actual ABI | | (such as a negative head CT) |
| | Should be monitored for | Between a range of | CT |
| | recovery from an actual ABI | about 17 to about 307 | (such as a positive head CT, a |
| | | | negative head CT, or no head |
| | | | CT) |
| | Sustained and is not | Greater than about | CT |
| | recovering from an actual ABI | 307 | (such as a positive head CT, a |
| | | | negative head CT, or no head |
| | | | CT) |
| 7 | Has not suffered or recovered | Less than about 18 | CT |
| | from an actual ABI | | (such as a negative head CT) |
| | Should be monitored for | Between a range of | CT |
| | recovery from an actual ABI | about 18 to about 294 | (such as a positive head CT, a |
| | | | negative head CT, or no head |
| | | | CT) |
| | Sustained and is not | Greater than about | CT |
| | recovering from an actual ABI | 294 | (such as a positive head CT, a |
| | | | negative head CT, or no head |
| | | | CT) |
| 8 | Has not suffered or recovered | Less than about 16 | CT |
| | from an actual ABI | | (such as a negative head CT) |
| | Should be monitored for | Between a range of | CT |
| | recovery from an actual ABI | about 16 to about 291 | (such as a positive head CT, a |
| | | | negative head CT, or no head |
| | | | CT) |
| | Sustained and is not | Greater than about | CT |
| | recovering from an actual ABI | 291 | (such as a positive head CT, a |
| | | | negative head CT, or no head |
| | | | CT) |

TABLE 5

| | | UCH-L1 level | |
|---|---|---|---|
| Option | Determining that a subject: | (pg/mL) | Measure |
| 1 | Has not suffered or recovered | Less than about 56 | GCS |
| | from an actual ABI | | (such as a mild TBI; GCS 13- |
| | | | 15) |
| | Should be monitored for | Between a range of | GCS |
| | recovery from an actual ABI | about 56 to about 267 | (such as a moderate, severe, |
| | | | or moderate-to-severe TBI; |
| | | | GCS 3-12) |
| | Sustained and is not | Greater than about | GCS |
| | recovering from an actual ABI | 267 | (such as a moderate, severe, |
| | | | or moderate-to-severe TBI; |
| | | | GCS 3-12) |
| 2 | Has not suffered or recovered | Less than about 61 | GCS |
| | from an actual ABI | | (such as a mild TBI; GCS 13- |
| | | | 15) |
| | Should be monitored for | Between a range of | GCS |
| | recovery from an actual ABI | about 61 to about 248 | (such as a moderate, severe, |
| | | | or moderate-to-severe TBI; |
| | | | GCS 3-12) |
| | Sustained and is not | Greater than about | GCS |
| | recovering from an actual ABI | 248 | (such as a moderate, severe, |
| | | | or moderate-to-severe TBI; |
| | | | GCS 3-12) |
| 3 | Has not suffered or recovered | Less than about 56 | GCS |
| | from an actual ABI | | (such as a mild TBI; GCS 13- |
| | | | 15) |
| | Should be monitored for | Between a range of | GCS |
| | recovery from an actual ABI | about 56 to about 250 | (such as a moderate, severe, |
| | | | or moderate-to-severe TBI; |
| | | | GCS 3-12) |
| | Sustained and is not | Greater than about | GCS |
| | recovering from an actual ABI | 250 | (such as a moderate, severe, |
| | | | or moderate-to-severe TBI; |
| | | | GCS 3-12) |
| 4 | Has not suffered or recovered | Less than about 49 | GOSE |
| | from an actual ABI | | (such as a GOSE score of 4-8) |
| | Should be monitored for | Between a range of | GOSE |
| | recovery from an actual ABI | about 49 to about 275 | (such as a GOSE score of 0-3) |
| | Sustained and is not | Greater than about | GOSE |
| | recovering from an actual ABI | 275 | (such as a GOSE score of 0-3) |
| 5 | Has not suffered or recovered | Less than about 63 | GOSE |
| | from an actual ABI | | (such as a GOSE score of 4-8) |
| | Should be monitored for | Between a range of | GOSE |
| | recovery from an actual ABI | about 63 to about 267 | (such as a GOSE score of 0-3) |
| | Sustained and is not | Greater than about | GOSE |
| | recovering from an actual ABI | 267 | (such as a GOSE score of 0-3) |
| 6 | Has not suffered or recovered | Less than about 53 | GOSE |
| | from an actual ABI | | (such as a GOSE score of 4-8) |
| | Should be monitored for | Between a range of | GOSE |
| | recovery from an actual ABI | about 53 to about 275 | (such as a GOSE score of 0-3) |
| | Sustained and is not | Greater than about | GOSE |
| | recovering from an actual ABI | 275 | (such as a GOSE score of 0-3) |
| 7 | Has not suffered or recovered | Less than about 55 | CT |
| | from an actual ABI | | (such as a negative head CT) |
| | Should be monitored for | Between a range of | CT |
| | recovery from an actual ABI | about 55 to about 250 | (such as a positive head CT, a |
| | | | negative head CT, or no head |
| | | | CT) |
| | Sustained and is not | Greater than about | CT |
| | recovering from an actual ABI | 250 | (such as a positive head CT, a |
| | | | negative head CT, or no head |
| | | | CT) |
| 8 | Has not suffered or recovered | Less than about 51 | CT |
| | from an actual ABI | | (such as a negative head CT) |
| | Should be monitored for | Between a range of | CT |
| | recovery from an actual ABI | about 51 to about 248 | (such as a positive head CT, a |
| | | | negative head CT, or no head |
| | | | CT) |
| | Sustained and is not | Greater than about | CT |
| | recovering from an actual ABI | 248 | (such as a positive head CT, a |
| | | | negative head CT, or no head |
| | | | CT) |
| 9 | Has not suffered or recovered | Less than about 55 | CT |
| | from an actual ABI | | (such as a negative head CT) |
| | Should be monitored for | Between a range of | CT |
| | recovery from an actual ABI | about 55 to about 243 | (such as a positive head CT, a |
| | | | negative head CT, or no head |
| | | | CT) |
| | Sustained and is not | Greater than about | CT |
| | recovering from an actual ABI | 243 | (such as a positive head CT, a |
| | | | negative head CT, or no head |
| | | | CT) |

TABLE 6

| | Determining that a | GFAP level | UCH-L1 level | |
|---|---|---|---|---|
| Option | subject: | (pg/mL) | (pg/mL) | Measure |
| 1 | Has not suffered or | Less than | Less than | Glasgow Coma |
| | recovered from an actual | about 18 | about 63 | Scale Score; |
| | ABI | pg/mL | pg/mL | Extended Glasgow |
| | | | | Outcome Scale |
| | | | | Score, a positive CT |
| | | | | scan for a TBI, a |
| | | | | negative scan for a |
| | | | | TBI or a |
| | | | | combination thereof |
| | Should be monitored for | Between about | Between about | Glasgow Coma |
| | recovery from an actual | 18 pg/mL at | 63 pg/mL at | Scale Score; |
| | ABI | the lower limit | the lower limit | Extended Glasgow |
| | | up to about | to about 275 | Outcome Scale |
| | | 431 pg/mL at | pg/mL at the | Score, a positive CT |
| | | the upper limit | upper limit | scan for a TBI, a |
| | | | | negative scan for a |
| | | | | TBI or a |
| | | | | combination thereof |
| | Sustained and is not | Greater than | Greater than | Glasgow Coma |
| | recovering from an actual | about 431 | about 275 | Scale Score; |
| | ABI | pg/mL | pg/mL | Extended Glasgow |
| | | | | Outcome Scale |
| | | | | Score, a positive CT |
| | | | | scan for a TBI, a |
| | | | | negative scan for a |
| | | | | TBI or a |
| | | | | combination thereof |
| 2 | Has not suffered or | Less than | Less than | A negative head CT |
| | recovered from an actual | about 18 | about 55 | scan |
| | ABI | pg/mL | pg/mL | |
| | Should be monitored for | Between about | Between about | A positive head CT |
| | recovery from an actual | 18 pg/mL to | 55 pg/mL to | scan or a negative |
| | ABI | about 307 | about 250 | head CT scan |
| | | pg/mL | pg/mL | |
| | Sustained and is not | Greater than | Greater than | A positive head CT |
| | recovering from an actual | about 307 | about 250 | scan |
| | ABI | pg/mL | pg/mL | |
| 3 | Has not suffered or | Less than | Less than | GCS score severity |
| | recovered from an actual | about 17 | about 61 | for a mild TBI (GCS |
| | ABI | pg/mL | pg/mL | 13-15) |
| | Should be monitored for | Between a | Between a | GCS score severity |
| | recovery from an actual | range of about | range of about | for a moderate, |
| | ABI | 17 pg/mL to | 61 pg/mL to | severe, or a |
| | | about 431 | about 267 | moderate-to-severe |
| | | pg/mL | pg/mL | TBI (GCS 3-12) |
| | Sustained and is not | Greater than | Greater than | GCS score severity |
| | recovering from an actual | about 431 | about 267 | for a moderate, |
| | ABI | pg/mL | pg/mL | severe, or a |
| | | | | moderate-to-severe |
| | | | | TBI (GCS 3-12) |
| 4 | Has not suffered or | Less than | Less than | GOSE score of 4-8 |
| | recovered from an actual | about 17 | about 63 | |
| | ABI | pg/mL | pg/mL | |
| | Should be monitored for | Between about | Between about | GOSE score of 0-3 |
| | recovery from an actual | 17 pg/mL to | 63 pg/mL to | |
| | ABI | about 431 | about 275 | |
| | | pg/mL | pg/mL | |
| | Sustained and is not | Greater than | Greater than | GOSE score of 0-3 |
| | recovering from an actual | about 431 | about 275 | |
| | ABI | pg/mL | pg/mL | |

In some embodiments, the method further includes treating and/or monitoring the subject, such as a human subject who is being monitored for recovery from an actual ABI and/or who has sustained an actual ABI and is not recovering from said subject, as described below.

The nature of the assay employed in the methods described herein is not critical and the test can be any assay known in the art such as, for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in a clinical chemistry format such as would be known by one of ordinary skill in the art. Such assays are described in further detail herein in Sections 4-8. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

In some embodiments, the methods described herein can be implemented by a computer.

In yet further embodiments, the present disclosure relates to a computer-implemented method for evaluation of a subject that has sustained or may have sustained an acquired brain injury (ABI). The method comprises:
a. obtaining data of a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI;
b. analyzing the data to obtain:
i) a first evaluation result if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) a second evaluation result if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) a third evaluation result if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL; and
outputting the first evaluation result, second evaluation result, or third evaluation result,
wherein the first evaluation result is associated with determining that the subject has not suffered or has recovered from an actual ABI; the second evaluation result is associated with determining that the subject should be monitored for recovery from an actual ABI; the third evaluation result is associated with determining that the subject sustained and is not recovering from an actual ABI.

In still other aspects of the above computer-implemented method:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects of the above computer-implemented method, the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

In still further aspects of the above computer-implemented method:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Moreover, in some aspects of the above, (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

In still further aspects of the above computer-implemented method:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL, and further wherein the GFAP level, the UCH-L1 level.

Moreover, in some aspects of the above, (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

In yet further aspects of the above computer-implemented method:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Moreover, in some aspects of the above, (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

In still other aspects of the above computer-implemented method, measuring the GFAP level, UCH-L1 level, or GFAP level and UCH-L1 level is done by immunoassay or a clinical chemistry assay. In some aspects the measuring of the GFAP level, UCH-L1 level, or GFAP level and UCH-L1 level is done by immunoassay. In still other aspects, the measuring of the UCH-L1 level is done by a clinical chemistry assay.

In still further aspects of the above computer-implemented method, the assay is a point-of-care assay. In still further aspects of the above computer-implemented method, the assay is a single molecule detection assay.

In still further aspects of the above computer-implemented method, the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

In still yet further aspects of the above computer-implemented method, the actual or suspected ABI is caused by physical shaking, blunt impact by an external mechanical or other force that results in a closed or open head trauma, one or more falls, explosions or blasts or other types of blunt force trauma. Specifically, in some aspects, the actual or suspected ABI is a traumatic brain injury. In yet further aspects, the actual or suspected ABI is a subacute ABI. In other aspects, when the actual or suspected ABI is a traumatic brain injury, the traumatic brain injury is a subacute traumatic brain injury.

In yet further aspects of the above computer-implemented method, the actual or suspected ABI is caused by fire exposure, ingestion of a chemical or toxin, or a combination of fire exposure, and ingestion of a chemical or toxin. Specifically, the chemical or toxin is mold, asbestos, a pesticide, an insecticide, an organic solvent, a paint, a glue, a gas, an organic metal, a drug of abuse or one or more combinations thereof.

In still further aspects of the above computer-implemented method, the actual or suspected ABI is caused by an autoimmune disease, a metabolic disorder, a brain tumor, hypoxia, a viral infection, a fungal infection, a bacterial infection, meningitis, hydrocephalus, or any combinations thereof.

In yet further aspects of the above computer-implemented method, the blood sample is whole blood, serum or plasma, either venous blood or capillary blood. In yet further aspects, the blood sample is obtained from venous blood and is whole blood, serum, or plasma. In still yet other aspects, blood sample is obtained from capillary blood and is whole blood, serum, or plasma.

In still further aspects of the above computer-implemented method, the subject is a human subject.

### 3. Apparatus, Non-Transitory Machine-Readable Storage Medium, and Systems

In some aspects, disclosed herein are apparatus, machine-readable storage mediums and systems for use in implementing or performing the methods described in Section 2. More specifically, the apparatus, machine-readable storage mediums and systems described herein can be used to identify GFAP, UCH-L1, or GFAP and UCH-L1 in one or more biological samples obtained from a subject, determine the level of GFAP, UCH-L1 or GFAP and UCH-L1 in the one or more biological samples, and communicate from an apparatus (e.g., such as a point-of-care, non-point-of-care, or a point-of-care or non-point-of-care apparatus), whether the level of GFAP, UCH-L1 or GFAP and UCH-L1, is greater than, less than, or between one or more reference or other levels. The term “apparatus” is used interchangeably with “device” or “instrument”.

In another aspect, the present disclosure relates to an apparatus, device, or instrument. The apparatus, device, or instrument contains software to execute one or more tasks, including the performance of the methods described in Section 2. In some aspects, the apparatus, device, or instrument contains software to automatically determine the next appropriate step in a methods described herein. For example, the apparatus, device, or instrument may contain software that determines the amount or presence of an analyte of interest. The software may display this determination, such as on a graphical user interface.

In some aspects, the apparatus, device, or instruments stores software that instructs processor or processor circuitry to execute or instantiate a given task. In some aspects, the software stores machine-readable instructions that cause processor circuitry to execute or instantiate a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or non-transitory machine-readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term “non-transitory computer readable medium” is defined to include any type of computer readable storage device and/or storage disk and to exclude propagating signals and to exclude transmission media.

In some further aspects, disclosed herein is a system for evaluation (also referred to herein as an “evaluation system”) of a subject that has sustained or may have sustained an acquired brain injury (ABI). The system comprises:
a. a data-obtaining module to obtain data of a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI;
b. an evaluation module to analyze the data to obtain:
i) a first evaluation result if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) a second evaluation result if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) a third evaluation result if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL; and
c. a data-outputting module to output the evaluation result (e.g., the first evaluation results, the second evaluation result or the third evaluation result),
wherein the first evaluation result is associated with determining that the subject has not suffered or has recovered from an actual ABI; the second evaluation result is associated with determining that the subject should be monitored for recovery from an actual ABI; and the third evaluation result is associated with determining that the subject sustained and is not recovering from an actual ABI.

In still other aspects of the evaluation system:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects of the above evaluation system, the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

In still further aspects of the evaluation system:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Moreover, in some aspects of the above, (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

In still further aspects of the above evaluation system:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL, and further wherein the GFAP level, the UCH-L1 level.

Moreover, in some aspects of the above, (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

In yet further aspects of the evaluation system:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Moreover, in some aspects of the above, (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

In still other aspects of the above evaluation system, measuring the GFAP level, UCH-L1 level, or GFAP level and UCH-L1 level is done by immunoassay. In still further aspects of the above evaluation system, measuring the GFAP level, UCH-L1 level, or GFAP level and UCH-L1 level is done by the GFAP level, UCH-L1 level, or GFAP level and UCH-L1 level is done by a clinical chemistry assay.

In still further aspects of the above evaluation system, the assay is a point-of-care assay or single molecule detection.

In still further aspects of the above evaluation system, the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

In still yet further aspects of the above evaluation system, the actual or suspected ABI is caused by physical shaking, blunt impact by an external mechanical or other force that results in a closed or open head trauma, one or more falls, explosions or blasts or other types of blunt force trauma. Specifically, in some aspects, the actual or suspected ABI is a traumatic brain injury. In yet further aspects, the actual or suspected ABI is a subacute ABI. In other aspects, when the actual or suspected ABI is a traumatic brain injury, the traumatic brain injury is a subacute traumatic brain injury.

In yet further aspects of the above evaluation system, the actual or suspected ABI is caused by fire exposure, ingestion of a chemical or toxin, or a combination of fire exposure, and ingestion of a chemical or toxin. Specifically, the chemical or toxin is mold, asbestos, a pesticide, an insecticide, an organic solvent, a paint, a glue, a gas, an organic metal, a drug of abuse or one or more combinations thereof.

In still further aspects of the above evaluation system, the actual or suspected ABI is caused by an autoimmune disease, a metabolic disorder, a brain tumor, hypoxia, a viral infection, a fungal infection, a bacterial infection, meningitis, hydrocephalus, or any combinations thereof.

In yet further aspects of the above evaluation system, the blood sample is whole blood, serum or plasma, either venous blood or capillary blood. In yet further aspects, the blood sample is obtained from venous blood and is whole blood, serum, or plasma. In still yet other aspects, blood sample is obtained from capillary blood and is whole blood, serum, or plasma.

In still further aspects of the above evaluation system, the subject is a human subject.

In further aspects, disclosed herein is a computer device that comprises a storage device having machine-readable instructions stored thereon, and a processor. The processor executes the machine-readable instructions to perform the steps of the above computer-implemented method or the steps performed by the above evaluation system.

In further aspects, disclosed herein is an apparatus, device, or instrument that comprises a storage device having machine-readable instructions stored thereon, and a processor. The processor instantiates or executes the machine-readable instructions to:
(A) identify GFAP (as a first biomarker), identify UCH-L1 (as a second biomarker), or both GFAP and UCH-L1 (as first and second biomarkers, respectively) in at least one or more biological samples obtained from a subject using at least one assay, where the at least one or more samples is obtained from the subject within after about twenty-four hours to (i) about one week, (ii) about two weeks, or (iii) about three weeks, after an actual or suspected ABI;
(B) determine a level of GFAP in the one or more samples, determine a level of UCH-L1 in one or more samples, or determine a level of GFAP in one or more samples and a level of UCH-L1 in one or more samples, and
(C) communicate from the apparatus, device, or instrument (e.g., such as displaying on the apparatus, device, or instrument) whether the subject's level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

In some aspects, the communication from the apparatus, device, or instrument is whether the subject's:
(A) (a) GFAP level is less than about 16 pg/mL; (b) UCH-L1 level is less than about 49 pg/mL; or (c) GFAP level is less than about 16 pg/mL and UCH-L1 level is less than about 49 pg/mL;
(B) (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
(C) (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects, the communication from the apparatus, device, or instrument is whether the subject's:
(A) (a) GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
(B) (a) GFAP level is between a range of about 16 pg/mL to about 291 pg/mL, about 17 pg/mL to about 307 pg/mL, about 18 pg/mL to about 294 pg/mL; (b) UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
(C) (a) GFAP level is greater than about 291 pg/mL, about 294 pg/mL, or about 307 pg/mL; (b) UCH-L1 level is greater than about 243 pg/mL, about 248 pg/mL, or about 250 pg/mL; (c) GFAP level is greater than about 291 pg/mL, about 294 pg/mL, or about 307 pg/mL, and UCH-L1 level is greater than about 243 pg/mL, about 248 pg/mL, or about 250 pg/mL.

In yet still further aspects, the communication from the apparatus, device, or instrument is whether the subject's:
(A) (a) GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
(B) (a) GFAP level is between a range of about 17 pg/mL to about 415 pg/mL, about 17 pg/mL to about 425 pg/mL, or about 17 pg/mL to about 431 pg/mL; (b) UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
(C) (a) GFAP level is greater than about 415 pg/mL, about 425 pg/mL, or about 431 pg/mL; (b) UCH-L1 level is greater than about 248 pg/mL, about 250 pg/mL, or about 267 pg/mL; (c) GFAP level is greater than about 415 pg/mL, about 425 pg/mL, or about 431 pg/mL and UCH-L1 level is greater than 248 pg/mL, about 250 pg/mL, or about 267 pg/mL.

In still yet further aspects, the communication from the apparatus, device, or instrument is whether the subject's:
(A) (a) GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
(B) (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
(C) (a) GFAP level is greater than about 375 pg/mL or about 431 pg/mL; (b) UCH-L1 level is greater than about 275 pg/mL or about 267 pg/mL; (c) GFAP level is greater than about 375 pg/mL or about 431 pg/mL and UCH-L1 level is greater than about 275 pg/mL or about 267 pg/mL.

In further aspects, disclosed herein is a non-transitory machine-readable storage medium having machine-readable instructions stored thereon, wherein the machine-readable instructions are configured to be executed by a processor to perform the steps of the above described computer-implemented method or the steps performed by the above one or more systems, such as the evaluation system.

In yet further aspects, disclosed herein is a non-transitory machine-readable storage medium. The non-transitory machine-readable storage medium can comprise instructions to cause one or processors or processor circuitry to at least:
(A) identify GFAP (as a first biomarker), identify UCH-L1 (as a second biomarker), or both GFAP and UCH-L1 (as first and second biomarkers, respectively) in at least one or more biological samples obtained from a subject using at least one assay, where the at least one or more samples is obtained from the subject within after about twenty-four hours to (i) about one week, (ii) about two weeks, or (iii) about three weeks, after an actual or suspected ABI;
(B) determine a level of GFAP in the one or more samples, determine a level of UCH-L1 in one or more samples, or determine a level of GFAP in one or more samples and a level of UCH-L1 in one or more samples, and
(C) communicate from an apparatus, device, or instrument (e.g., such as displaying on the apparatus, device, or instrument) whether the subject's level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

In some aspects, the communication from an apparatus, device, or instrument is whether the subject's:
(A) (a) GFAP level is less than about 16 pg/mL; (b) UCH-L1 level is less than about 49 pg/mL; or (c) GFAP level is less than about 16 pg/mL and UCH-L1 level is less than about 49 pg/mL;
(B) (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
(C) (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects, the communication from an apparatus, device, or instrument is whether the subject's:
(A) (a) GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
(B) (a) GFAP level is between a range of about 16 pg/mL to about 291 pg/mL, about 17 pg/mL to about 307 pg/mL, about 18 pg/mL to about 294 pg/mL; (b) UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
(C) (a) GFAP level is greater than about 291 pg/mL, about 294 pg/mL, or about 307 pg/mL; (b) UCH-L1 level is greater than about 243 pg/mL, about 248 pg/mL, or about 250 pg/mL; (c) GFAP level is greater than about 291 pg/mL, about 294 pg/mL, or about 307 pg/mL, and UCH-L1 level is greater than about 243 pg/mL, about 248 pg/mL, or about 250 pg/mL.

In yet still further aspects, the communication from an apparatus, device, or instrument is whether the subject's:
(A) (a) GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
(B) (a) GFAP level is between a range of about 17 pg/mL to about 415 pg/mL, about 17 pg/mL to about 425 pg/mL, or about 17 pg/mL to about 431 pg/mL; (b) UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
(C) (a) GFAP level is greater than about 415 pg/mL, about 425 pg/mL, or about 431 pg/mL; (b) UCH-L1 level is greater than about 248 pg/mL, about 250 pg/mL, or about 267 pg/mL; (c) GFAP level is greater than about 415 pg/mL, about 425 pg/mL, or about 431 pg/mL and UCH-L1 level is greater than 248 pg/mL, about 250 pg/mL, or about 267 pg/mL.

In still yet further aspects, the communication from an apparatus, device, or instrument is whether the subject's:
(A) (a) GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
(B) (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
(C) (a) GFAP level is greater than about 375 pg/mL or about 431 pg/mL; (b) UCH-L1 level is greater than about 275 pg/mL or about 267 pg/mL; (c) GFAP level is greater than about 375 pg/mL or about 431 pg/mL and UCH-L1 level is greater than about 275 pg/mL or about 267 pg/mL.

In still yet further aspects disclosed herein are systems. In some aspects, the system comprises an apparatus, device, or instrument and certain instructions. With respect to the apparatus, the apparatus, device, or instrument comprises a storage device having machine readable instructions stored thereon and a processor or processor circuitry which instantiates or executes the machine-readable instructions to:
(A) identify GFAP (as a first biomarker), identify UCH-L1 (as a second biomarker), or both GFAP and UCH-L1 (as first and second biomarkers, respectively) in at least one or more biological samples obtained from a subject using at least one assay, where the at least one or more samples is obtained from the subject within after about twenty-four hours to (i) about one week, (ii) about two weeks, or (iii) about three weeks, after an actual or suspected ABI;
(B) determine a level of GFAP in the one or more samples, determine a level of UCH-L1 in one or more samples, or determine a level of GFAP in one or more samples and a level of UCH-L1 in one or more samples, and
(C) communicate from the apparatus, device, or instrument (e.g., such as displaying on the apparatus, device, or instrument) whether the subject's level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

In some aspects, the communication from the apparatus, device, or instrument is whether the subject's:
(A) (a) GFAP level is less than about 16 pg/mL; (b) UCH-L1 level is less than about 49 pg/mL; or (c) GFAP level is less than about 16 pg/mL and UCH-L1 level is less than about 49 pg/mL;
(B) (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
(C) (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects, the communication from the apparatus, device, or instrument is whether the subject's:
(A) (a) GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
(B) (a) GFAP level is between a range of about 16 pg/mL to about 291 pg/mL, about 17 pg/mL to about 307 pg/mL, about 18 pg/mL to about 294 pg/mL; (b) UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
(C) (a) GFAP level is greater than about 291 pg/mL, about 294 pg/mL, or about 307 pg/mL; (b) UCH-L1 level is greater than about 243 pg/mL, about 248 pg/mL, or about 250 pg/mL; (c) GFAP level is greater than about 291 pg/mL, about 294 pg/mL, or about 307 pg/mL, and UCH-L1 level is greater than about 243 pg/mL, about 248 pg/mL, or about 250 pg/mL.

In yet still further aspects, the communication from the apparatus, device, or instrument is whether the subject's:
(A) (a) GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
(B) (a) GFAP level is between a range of about 17 pg/mL to about 415 pg/mL, about 17 pg/mL to about 425 pg/mL, or about 17 pg/mL to about 431 pg/mL; (b) UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
(C) (a) GFAP level is greater than about 415 pg/mL, about 425 pg/mL, or about 431 pg/mL; (b) UCH-L1 level is greater than about 248 pg/mL, about 250 pg/mL, or about 267 pg/mL; (c) GFAP level is greater than about 415 pg/mL, about 425 pg/mL, or about 431 pg/mL and UCH-L1 level is greater than 248 pg/mL, about 250 pg/mL, or about 267 pg/mL.

In still yet further aspects, the communication from the apparatus, device, or instrument is whether the subject's:
(A) (a) GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
(B) (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
(C) (a) GFAP level is greater than about 375 pg/mL or about 431 pg/mL; (b) UCH-L1 level is greater than about 275 pg/mL or about 267 pg/mL; (c) GFAP level is greater than about 375 pg/mL or about 431 pg/mL and UCH-L1 level is greater than about 275 pg/mL or about 267 pg/mL.

In addition to the apparatus, device, or instrument, the system also comprises instructions. The instructions included in the system are for use in determining whether:
(A) the subject has not suffered or has recovered from an actual acquired brain injury (ABI) if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL; or
(B) the subject should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
(C) the subject has sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

In some aspects, the instructions provide determining whether the subject's:
(A) (a) GFAP level is less than about 16 pg/mL; (b) UCH-L1 level is less than about 49 pg/mL; or (c) GFAP level is less than about 16 pg/mL and UCH-L1 level is less than about 49 pg/mL;
(B) (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
(C) (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

In still further aspects, the instructions provide determining whether the subject's:
(A) (a) GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
(B) (a) GFAP level is between a range of about 16 pg/mL to about 291 pg/mL, about 17 pg/mL to about 307 pg/mL, about 18 pg/mL to about 294 pg/mL; (b) UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
(C) (a) GFAP level is greater than about 291 pg/mL, about 294 pg/mL, or about 307 pg/mL; (b) UCH-L1 level is greater than about 243 pg/mL, about 248 pg/mL, or about 250 pg/mL; (c) GFAP level is greater than about 291 pg/mL, about 294 pg/mL, or about 307 pg/mL, and UCH-L1 level is greater than about 243 pg/mL, about 248 pg/mL, or about 250 pg/mL.

In yet still further aspects, the instructions provide determining whether the subject's:
(A) (a) GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
(B) (a) GFAP level is between a range of about 17 pg/mL to about 415 pg/mL, about 17 pg/mL to about 425 pg/mL, or about 17 pg/mL to about 431 pg/mL; (b) UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
(C) (a) GFAP level is greater than about 415 pg/mL, about 425 pg/mL, or about 431 pg/mL; (b) UCH-L1 level is greater than about 248 pg/mL, about 250 pg/mL, or about 267 pg/mL; (c) GFAP level is greater than about 415 pg/mL, about 425 pg/mL, or about 431 pg/mL and UCH-L1 level is greater than 248 pg/mL, about 250 pg/mL, or about 267 pg/mL.

In still yet further aspects, the instructions provide determining whether the subject's:
(A) (a) GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
(B) (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
(C) (a) GFAP level is greater than about 375 pg/mL or about 431 pg/mL; (b) UCH-L1 level is greater than about 275 pg/mL or about 267 pg/mL; (c) GFAP level is greater than about 375 pg/mL or about 431 pg/mL and UCH-L1 level is greater than about 275 pg/mL or about 267 pg/mL.

The instructions that are included in the system can be provided as written instructions, such as in a product insert, product manual, excel document, etc., on a mobile device (e.g., a smart phone), on a website, in an e-mail, or any combination thereof.

### 4. Treatment and Monitoring of Subjects Who have Sustained an Injury to the Head

The subject identified in the methods described above may be treated or monitored. The subject (e.g., a human subject) identified or assessed in the methods described above as having an acquired brain injury, such as traumatic brain injury (e.g., a mild TBI, moderate TBI, severe TBI or moderate to severe TBI), may be treated or monitored. In some embodiments, the method further includes treating the subject (e.g., human subject (e.g., a human adult or human pediatric subject) determined as having a TBI with a traumatic brain injury treatment, such as any treatments known in the art. For example, treatment of traumatic brain injury can take a variety of forms depending on the severity of the injury to the head. For example, for subjects suffering from mild TBI, the treatment may include one or more of: (1) rest (e.g., physical and/or mental rest) (2) abstaining from physical activities such as sports, work, school, play, or any combinations thereof; (3) avoiding light or wearing sunglasses when out in the light; (4) administering one or more types of medication, such as, for example, medication for relief of a headache or migraine (e.g., steroidal anti-inflammatory drugs (e.g., corticosteroids (prednisone or dexamethasone)) or nonsteroidal anti-inflammatory drugs (NSAIDs, such as, aspirin, ibuprofen, naproxen sodium, etc.), medication to treat dizziness, anti-nausea medication (e.g., an anti-emetic), motion sickness medications (e.g., scopolamine, promethazine, dimenhydrinate, etc.), anti-depressant medication (e.g., a SSRI, such as fluoxetine (Prozac), paroxetine (Paxil), fluvoxamine (Luvox), citalopram (Celexa), escitalopram (Cipralex), sertraline (Zoloft), and/or psychedelic drugs, such as psilocybin and MDMA, etc.), anti-anxiety medication (e.g., a SSRI, such as fluoxetine (Prozac), paroxetine (Paxil), fluvoxamine (Luvox), citalopram (Celexa), escitalopram (Cipralex), sertraline (Zoloft), and/or psychedelic drugs, such as psilocybin, MDMA, etc.), sleep aid medication (e.g., melatonin, estazolam, flurazepam, quazepam, temazepam, triazolam, etc.), muscle relaxants (e.g., if there is injury or involvement of the neck or muscles around the neck in order to reduce inflammation that might be exacerbating a TBI), anti-inflammatory medications (e.g., steroidal anti-inflammatory drugs or NSAIDs), medications to improve concentration and/or focus (e.g., psychostimulants such as methylphenidate, etc.) and/or one or more naturopathic medicines or treatments (e.g., non-hallucigenic mushrooms, herbal tea, acupuncture, medical marijuana, etc.); (5) providing one or more devices to treat nausea, such as acupressure wrist bands (e.g., a Psi band); (6) administering one or more nutrachemicals and/or nutritional compositions that contain one or more omega-3 fatty acids, one or more vitamins (e.g., vitamin B and/or vitamin D), one or more fatty acids and/or one or more antioxidants; (7) providing hyperbaric oxygen therapy; (8) light therapy (i.e., to help with mood swings, irritability and/or depression resulting from a TBI); (9) physical therapy for TBI (e.g., to treat dizziness, improve motor skills, etc.); (10) occupational therapy for TBI (e.g., to help improve memory, concentration, focus, etc.); (11) individual, group counseling and/or psychotherapy to help treat depression, and/or anxiety resulting from a TBI; (12) sleep therapy to help treat sleep disorders (e.g., not sleeping or oversleeping) arising from a TBI; or (13) and any combinations of (1)-(12).

Treatment for patients suffering from moderate, severe or moderate-to-severe TBI might include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.), protecting the airway, and one or more therapies (such as, for example one or more rehabilitation, cognitive behavioral therapy, anger management, physical therapy, occupational therapy, a combination of physical and occupational therapy, counseling psychology, etc.). In some embodiments, the method further includes monitoring the subject, such as a human subject. In some embodiments, a subject may be monitored with CT scan or MRI procedure.

### 5. Methods for Measuring the Level of UCH-L1

In the methods described above, UCH-L1 levels can be measured by any means, such as antibody dependent methods, such as immunoassays, protein immunoprecipitation, immunoelectrophoresis, chemical analysis, SDS-PAGE and Western blot analysis, protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of UCH-L1 includes contacting the sample with a first specific binding member and second specific binding member. In some embodiments the first specific binding member is a capture antibody and the second specific binding member is a detection antibody. In some embodiments, measuring the level of UCH-L1 includes contacting the sample, either simultaneously or sequentially, in any order: (1) a capture antibody (e.g., UCH-L1-capture antibody), which binds to an epitope on UCH-L1 or UCH-L1 fragment to form a capture antibody-UCH-L1 antigen complex (e.g., UCH-L1-capture antibody-UCH-L1 antigen complex), and (2) a detection antibody (e.g., UCH-L1-detection antibody), which includes a detectable label and binds to an epitope on UCH-L1 that is not bound by the capture antibody, to form a UCH-L1 antigen-detection antibody complex (e.g., UCH-L1 antigen-UCH-L1-detection antibody complex), such that a capture antibody-UCH-L1 antigen-detection antibody complex (e.g., UCH-L1-capture antibody-UCH-L1 antigen-UCH-L1-detection antibody complex) is formed, and measuring the amount or concentration of UCH-L1 in the sample based on the signal generated by the detectable label in the capture antibody-UCH-L1 antigen-detection antibody complex.

In some embodiments, the first specific binding member is immobilized on a solid support. In some embodiments, the second specific binding member is immobilized on a solid support. In some embodiments, the first specific binding member is a UCH-L1 antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instrument ARCHITECT, and other core laboratory instruments) other than a point-of-care device may be capable of measuring levels of UCH-L1 in a sample higher or greater than 25,000 pg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 6. UCH-L1 Antibodies

The methods described herein may use an isolated antibody that specifically binds to ubiquitin carboxy-terminal hydrolase L1 (“UCH-L1”) (or fragments thereof), referred to as “UCH-L1 antibody.” The UCH-L1 antibodies can be used to assess the UCH-L1 status as a measure of traumatic brain injury, detect the presence of UCH-L1 in a sample, quantify the amount of UCH-L1 present in a sample, or detect the presence of and quantify the amount of UCH-L1 in a sample.

a. Ubiquitin Carboxy-Terminal Hydrolase L1 (UCH-L1)

Ubiquitin carboxy-terminal hydrolase L1 (“UCH-L1”), which is also known as “ubiquitin C-terminal hydrolase,” is a deubiquitinating enzyme. UCH-L1 is a member of a gene family whose products hydrolyze small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary. The catalytic triad of UCH-L1 contains a cysteine at position 90, an aspartate at position 176, and a histidine at position 161 that are responsible for its hydrolase activity.

Human UCH-L1 may have the following amino acid sequence:

(SEQ ID NO: 1)
MQLKPMEINPEMLNKVLSRLGVAGQWRFVDVLGLEEESLGSVPAPACAL
LLLFPLTAQHENFRKKQIEELKGQEVSPKVYFMKQTIGNSCGTIGLIHA
VANNQDKLGFEDGSVLKQFLSETEKMSPEDRAKCFEKNEAIQAAHDAVA
QEGQCRVDDKVNFHFILENNVDGHLYELDGRMPFPVNHGASSEDTLLKD
AAKVCREFTEREQGEVRESAVALCKAA.

The human UCH-L1 may be a fragment or variant of SEQ ID NO: 1. The fragment of UCH-L1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise a contiguous number of amino acids from SEQ ID NO: 1.

b. UCH-L1-Recognizing Antibody

The antibody is an antibody that binds to UCH-L1, a fragment thereof, an epitope of UCH-L1, or a variant thereof. The antibody may be a fragment of the anti-UCH-L1 antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-UCH-L1 antibodies may be a chimeric anti-UCH-L1 or humanized anti-UCH-L1 antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-UCH-L1 antibodies. For example, libraries of naïve human single chain variable fragments (scFv) may be used to select human anti-UCH-L1 antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to UCH-L1 (SEQ ID NO: 1), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human UCH-L1) and the other heavy and light chain are specific for an antigen other than human UCH-L1 by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with UCH-L1 or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See, U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al., J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. Sec, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two-fold stronger than the binding affinity of the parent antibody for the antigen.

The antibody may be a linear antibody. The procedure for making a linear antibody is known in the art and described in Zapata et al., (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F (ab).

Antibody production via the use of hybridoma technology, the selected lymphocyte antibody method (SLAM), transgenic animals, and recombinant antibody libraries is described in more detail below.

### (1) Anti-UCH-L1 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the present disclosure wherein, preferably, the hybridoma is generated by fusing splenocytes isolated from an animal, e.g., a rat or a mouse, immunized with UCH-L1 with myeloma cells and then screening the hybridomas resulting from the fusion for hybridoma clones that secrete an antibody able to bind a polypeptide of the present disclosure. Briefly, rats can be immunized with a UCH-L1 antigen. In a preferred embodiment, the UCH-L1 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a UCH-L1 antigen, antibodies and/or antibody-producing cells may be obtained from the animal. An anti-UCH-L1 antibody-containing serum is obtained from the animal by bleeding or sacrificing the animal. The serum may be used as it is obtained from the animal, an immunoglobulin fraction may be obtained from the serum, or the anti-UCH-L1 antibodies may be purified from the serum. Serum or immunoglobulins obtained in this manner are polyclonal, thus having a heterogeneous array of properties.

Once an immune response is detected, e.g., antibodies specific for the antigen UCH-L1 are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding UCH-L1. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using UCH-L1, or a portion thereof, or a cell expressing UCH-L1. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-UCH-L1 antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-UCH-L1 antibody.

Antibody fragments that recognize specific epitopes may be generated by known techniques. For example, Fab and F(ab′)₂ fragments of the present disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-UCH-L1 Monoclonal Antibodies Using SLAM

In another aspect of the present disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen UCH-L1, a subunit of UCH-L1, or a fragment thereof, is coupled to sheep red blood cells using a linker, such as biotin, and used to identify single cells that secrete antibodies with specificity for UCH-L1. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to UCH-L1. The amplified immunoglobulin sequences further can be manipulated in vitro, such as by in vitro affinity maturation method. Sec, for example, PCT Publication No. WO 97/29131 and PCT Publication No. WO 00/56772.

### (3) Anti-UCH-L1 Monoclonal Antibodies Using Transgenic Animals

In another embodiment of the present disclosure, antibodies are produced by immunizing a non-human animal comprising some, or all, of the human immunoglobulin locus with a UCH-L1 antigen. In an embodiment, the non-human animal is a XENOMOUSE® transgenic mouse, an engineered mouse strain that comprises large fragments of the human immunoglobulin loci and is deficient in mouse antibody production. Sec, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. Sec Mendez et al., Nature Genetics, 15:146-156 (1997), Green and Jakobovits, J. Exp. Med., 188:483-495 (1998), the disclosures of which are hereby incorporated by reference.

### (4) Anti-UCH-L1 Monoclonal Antibodies Using Recombinant Antibody Libraries

In vitro methods also can be used to make the antibodies of the present disclosure, wherein an antibody library is screened to identify an antibody having the desired UCH-L1-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with UCH-L1, or a portion of UCH-L1. Alternatively, the recombinant antibody library may be from a naive subject, i.e., one who has not been immunized with UCH-L1, such as a human antibody library from a human subject who has not been immunized with human UCH-L1. Antibodies of the present disclosure are selected by screening the recombinant antibody library with the peptide comprising human UCH-L1 to thereby select those antibodies that recognize UCH-L1. Methods for conducting such screening and selection are well known in the art, such as described in the references in the preceding paragraph. To select antibodies of the present disclosure having particular binding affinities for UCH-L1, such as those that dissociate from human UCH-L1 with a particular K_{off} rate constant, the art-known method of surface plasmon resonance can be used to select antibodies having the desired K_{off} rate constant. To select antibodies of the present disclosure having a particular neutralizing activity for hUCH-L1, such as those with a particular IC₅₀, standard methods known in the art for assessing the inhibition of UCH-L1 activity may be used.

In one aspect, the present disclosure pertains to an isolated antibody, or an antigen-binding portion thereof, that binds human UCH-L1. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the present disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant UCH-L1 Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the present disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the present disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this present disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the present disclosure. In addition, bifunctional antibodies may be produced in which one heavy and one light chain are an antibody of the present disclosure (i.e., binds human UCH-L1) and the other heavy and light chain are specific for an antigen other than human UCH-L1 by crosslinking an antibody of the present disclosure to a second antibody by standard chemical crosslinking methods.

In a preferred system for recombinant expression of an antibody, or antigen-binding portion thereof, of the present disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the present disclosure provides a method of synthesizing a recombinant antibody of the present disclosure by culturing a host cell of the present disclosure in a suitable culture medium until a recombinant antibody of the present disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for UCH-L1 and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for UCH-L1, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-UCH-L1 Antibodies

Anti-UCH-L1 antibodies may be generated using the techniques described above as well as using routine techniques known in the art. In some embodiments, the anti-UCH-L1 antibody may be an unconjugated UCH-L1 antibody, such as UCH-L1 antibodies available from United State Biological (Catalog Number: 031320), Cell Signaling Technology (Catalog Number: 3524), Sigma-Aldrich (Catalog Number: HPA005993), Santa Cruz Biotechnology, Inc. (Catalog Numbers: sc-58593 or sc-58594), R&D Systems (Catalog Number: MAB6007), Novus Biologicals (Catalog Number: NB600-1160), Biorbyt (Catalog Number: orb33715), Enzo Life Sciences, Inc. (Catalog Number: ADI-905-520-1), Bio-Rad (Catalog Number: VMA00004), Bio Vision (Catalog Number: 6130-50), Abcam (Catalog Numbers: ab75275 or ab104938), Invitrogen Antibodies (Catalog Numbers: 480012), ThermoFisher Scientific (Catalog Numbers: MA1-46079, MA5-17235, MA1-90008, or MA1-83428), EMD Millipore (Catalog Number: MABN48), or Sino Biological Inc. (Catalog Number: 50690-R011). The anti-UCH-L1 antibody may be conjugated to a fluorophore, such as conjugated UCH-L1 antibodies available from BioVision (Catalog Number: 6960-25) or Aviva Systems Biology (Cat. Nos. OAAF01904-FITC).

Alternatively, the antibodies described in WO2018081649, U.S. Pat. No. 11,078,298, WO 2018/067468 and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 7. Methods for Measuring the Level of GFAP

In the methods described above, GFAP levels can be measured by any means, such as antibody dependent methods, such as immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of GFAP includes contacting the sample with a first specific binding member and second specific binding member. In some embodiments the first specific binding member is a capture antibody and the second specific binding member is a detection antibody. In some embodiments, measuring the level of GFAP includes contacting the sample, either simultaneously or sequentially, in any order, with: (1) a capture antibody (e.g., GFAP-capture antibody), which binds to an epitope on GFAP or GFAP fragment to form a capture antibody-GFAP antigen complex (e.g., GFAP-capture antibody-GFAP antigen complex), and (2) a detection antibody (e.g., GFAP-detection antibody), which includes a detectable label and binds to an epitope on GFAP that is not bound by the capture antibody, to form a GFAP antigen-detection antibody complex (e.g., GFAP antigen-GFAP-detection antibody complex), such that a capture antibody-GFAP antigen-detection antibody complex (e.g., GFAP-capture antibody-GFAP antigen-GFAP-detection antibody complex) is formed, and measuring the amount or concentration of GFAP in the sample based on the signal generated by the detectable label in the capture antibody-GFAP antigen-detection antibody complex.

In some embodiments, the first specific binding member is immobilized on a solid support. In some embodiments, the second specific binding member is immobilized on a solid support. In some embodiments, the first specific binding member is a GFAP antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instrument ARCHITECT, Alinity, and other core laboratory instruments) other than a point-of-care device may be capable of measuring levels of GFAP in a sample higher or greater than 25,000 pg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 8. GFAP Antibodies

The methods described herein may use an isolated antibody that specifically binds to Glial fibrillary acidic protein (“GFAP”) (or fragments thereof), referred to as “GFAP antibody.” The GFAP antibodies can be used to assess the GFAP status as a measure of traumatic brain injury, detect the presence of GFAP in a sample, quantify the amount of GFAP present in a sample, or detect the presence of and quantify the amount of GFAP in a sample.

a. Glial Fibrillary Acidic Protein (GFAP)

Glial fibrillary acidic protein (GFAP) is a 50 kDa intracytoplasmic filamentous protein that constitutes a portion of the cytoskeleton in astrocytes, and it has proved to be the most specific marker for cells of astrocytic origin. GFAP protein is encoded by the GFAP gene in humans. GFAP is the principal intermediate filament of mature astrocytes. In the central rod domain of the molecule, GFAP shares considerable structural homology with the other intermediate filaments. GFAP is involved in astrocyte motility and shape by providing structural stability to astrocytic processes. Glial fibrillary acidic protein and its breakdown products (GFAP-BDP) are brain-specific proteins released into the blood as part of the pathophysiological response after traumatic brain injury (TBI). Following injury to the human CNS caused by trauma, genetic disorders, or chemicals, astrocytes proliferate and show extensive hypertrophy of the cell body and processes, and GFAP is markedly upregulated. In contrast, with increasing astrocyte malignancy, there is a progressive loss of GFAP production. GFAP can also be detected in Schwann cells, enteric glia cells, salivary gland neoplasms, metastasizing renal carcinomas, epiglottic cartilage, pituicytes, immature oligodendrocytes, papillary meningiomas, and myoepithelial cells of the breast.

Human GFAP may have the following amino acid sequence:

(SEQ ID NO: 2)
MERRRITSAARRSYVSSGEMMVGGLAPGRRLGPGTRLSLARMPPPLPTR
VDFSLAGALNAGFKETRASERAEMMELNDRFASYIEKVRFLEQQNKALA
AELNQLRAKEPTKLADVYQAELRELRLRLDQLTANSARLEVERDNLAQD
LATVRQKLQDETNLRLEAENNLAAYRQEADEATLARLDLERKIESLEEE
IRFLRKIHEEEVRELQEQLARQQVHVELDVAKPDLTAALKEIRTQYEAM
ASSNMHEAEEWYRSKFADLTDAAARNAELLRQAKHEANDYRRQLQSLTC
DLESLRGTNESLERQMREQEERHVREAASYQEALARLEEEGQSLKDEMA
RHLQEYQDLLNVKLALDIEIATYRKLLEGEENRITIPVQTFSNLQIRET
SLDTKSVSEGHLKRNIVVKTVEMRDGEVIKESKQEHKDVM.

The human GFAP may be a fragment or variant of SEQ ID NO: 2. The fragment of GFAP may be between 5 and 400 amino acids, between 10 and 400 amino acids, between 50 and 400 amino acids, between 60 and 400 amino acids, between 65 and 400 amino acids, between 100 and 400 amino acids, between 150 and 400 amino acids, between 100 and 300 amino acids, or between 200 and 300 amino acids in length. The fragment may comprise a contiguous number of amino acids from SEQ ID NO: 2. The human GFAP fragment or variant of SEQ ID NO: 2 may be a GFAP breakdown product (BDP). The GFAP BDP may be 38 kDa, 42 kDa (fainter 41 kDa), 47 kDa (fainter 45 kDa); 25 kDa (fainter 23 kDa); 19 kDa, or 20 kDa.

b. GFAP-Recognizing Antibody

The antibody is an antibody that binds to GFAP, a fragment thereof, an epitope of GFAP, or a variant thereof. The antibody may be a fragment of the anti-GFAP antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-GFAP antibodies may be a chimeric anti-GFAP or humanized anti-GFAP antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-GFAP antibodies. For example, libraries of naïve human single chain variable fragments (scFv) may be used to select human anti-GFAP antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to GFAP (SEQ ID NO: 2), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human GFAP) and the other heavy and light chain are specific for an antigen other than human GFAP by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with GFAP or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH: VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F (ab).

Antibody production via the use of hybridoma technology, the selected lymphocyte antibody method (SLAM), transgenic animals, and recombinant antibody libraries is described in more detail below.

### (1) Anti-GFAP Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the present disclosure wherein, preferably, the hybridoma is generated by fusing splenocytes isolated from an animal, e.g., a rat or a mouse, immunized with GFAP with myeloma cells and then screening the hybridomas resulting from the fusion for hybridoma clones that secrete an antibody able to bind a polypeptide of the present disclosure. Briefly, rats can be immunized with a GFAP antigen. In a preferred embodiment, the GFAP antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a GFAP antigen, antibodies and/or antibody-producing cells may be obtained from the animal. An anti-GFAP antibody-containing serum is obtained from the animal by bleeding or sacrificing the animal. The serum may be used as it is obtained from the animal, an immunoglobulin fraction may be obtained from the serum, or the anti-GFAP antibodies may be purified from the serum. Serum or immunoglobulins obtained in this manner are polyclonal, thus having a heterogeneous array of properties.

Once an immune response is detected, e.g., antibodies specific for the antigen GFAP are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding GFAP. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. Sec, e.g., Harlow and Lane, supra. In a preferred embodiment, the myeloma cells do not secrete immunoglobulin polypeptides (a non-secretory cell line). After fusion and antibiotic selection, the hybridomas are screened using GFAP, or a portion thereof, or a cell expressing GFAP. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-GFAP antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-GFAP antibody.

Antibody fragments that recognize specific epitopes may be generated by known techniques. For example, Fab and F(ab′)₂ fragments of the present disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-GFAP Monoclonal Antibodies Using SLAM

In another aspect of the present disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen GFAP, a subunit of GFAP, or a fragment thereof, is coupled to sheep red blood cells using a linker, such as biotin, and used to identify single cells that secrete antibodies with specificity for GFAP. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to GFAP. The amplified immunoglobulin sequences further can be manipulated in vitro, such as by in vitro affinity maturation method. See, for example, PCT Publication No. WO 97/29131 and PCT Publication No. WO 00/56772.

### (3) Anti-GFAP Monoclonal Antibodies Using Transgenic Animals

In another embodiment of the present disclosure, antibodies are produced by immunizing a non-human animal comprising some, or all, of the human immunoglobulin locus with a GFAP antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

### (4) Anti-GFAP Monoclonal Antibodies Using Recombinant Antibody Libraries

In vitro methods also can be used to make the antibodies of the present disclosure, wherein an antibody library is screened to identify an antibody having the desired GFAP-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with GFAP, or a portion of GFAP. Alternatively, the recombinant antibody library may be from a naive subject, i.e., one who has not been immunized with GFAP, such as a human antibody library from a human subject who has not been immunized with human GFAP. Antibodies of the present disclosure are selected by screening the recombinant antibody library with the peptide comprising human GFAP to thereby select those antibodies that recognize GFAP. Methods for conducting such screening and selection are well known in the art, such as described in the references in the preceding paragraph. To select antibodies of the present disclosure having particular binding affinities for GFAP, such as those that dissociate from human GFAP with a particular K_{off} rate constant, the art-known method of surface plasmon resonance can be used to select antibodies having the desired K_{off} rate constant. To select antibodies of the present disclosure having a particular neutralizing activity for GFAP, such as those with a particular IC₅₀, standard methods known in the art for assessing the inhibition of GFAP activity may be used.

In one aspect, the present disclosure pertains to an isolated antibody, or an antigen-binding portion thereof, that binds human GFAP. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the present disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant GFAP Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the present disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the present disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this present disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the present disclosure. In addition, bifunctional antibodies may be produced in which one heavy and one light chain are an antibody of the present disclosure (i.e., binds human GFAP) and the other heavy and light chain are specific for an antigen other than human GFAP by crosslinking an antibody of the present disclosure to a second antibody by standard chemical crosslinking methods.

In a preferred system for recombinant expression of an antibody, or antigen-binding portion thereof, of the present disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the present disclosure provides a method of synthesizing a recombinant antibody of the present disclosure by culturing a host cell of the present disclosure in a suitable culture medium until a recombinant antibody of the present disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for GFAP and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for GFAP, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g. mice that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-GFAP Antibodies

Anti-GFAP antibodies may be generated using the techniques described above as well as using routine techniques known in the art. In some embodiments, the anti-GFAP antibody may be an unconjugated GFAP antibody, such as GFAP antibodies available from Dako (Catalog Number: M0761), ThermoFisher Scientific (Catalog Numbers: MA5-12023, A-21282, 13-0300, MAI-19170, MAI-19395, MA5-15086, MA5-16367, MAI-35377, MAI-06701, or MAI-20035), AbCam (Catalog Numbers: ab10062, ab4648, ab68428, ab33922, ab207165, ab190288, ab115898, or ab21837), EMD Millipore (Catalog Numbers: FCMAB257P, MAB360, MAB3402, 04-1031, 04-1062, MAB5628), Santa Cruz (Catalog Numbers: sc-166481, sc-166458, sc-58766, sc-56395, sc-51908, sc-135921, sc-71143, sc-65343, or sc-33673), Sigma-Aldrich (Catalog Numbers: G3893 or G6171) or Sino Biological Inc. (Catalog Number: 100140-R012-50). The anti-GFAP antibody may be conjugated to a fluorophore, such as conjugated GFAP antibodies available from ThermoFisher Scientific (Catalog Numbers: A-21295 or A-21294), EMD Millipore (Catalog Numbers: MAB3402X, MAB3402B, MAB3402B, or MAB3402C3) or AbCam (Catalog Numbers: ab49874 or ab194325).

Alternatively, the antibodies described in WO2018081649, U.S. Pat. No. 11,078,298, WO 2018/067474 and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 9. Variations on Methods

The disclosed methods of determining the presence or amount of analyte of interest (UCH-L1 and/or GFAP) present in a sample may be as described herein. The methods may also be adapted in view of other methods for analyzing analytes. Examples of well-known variations include, but are not limited to, immunoassay, such as sandwich immunoassay (e.g., monoclonal-monoclonal sandwich immunoassays, monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc.

a. Immunoassay

The analyte of interest, and/or peptides of fragments thereof (e.g., UCH-L1 and/or GFAP, and/or peptides or fragments thereof, i.e., UCH-L1 and/or GFAP fragments), may be analyzed using UCH-L1 and/or GFAP antibodies in an immunoassay. The presence or amount of analyte (e.g., UCH-L1 and/or GFAP) can be determined using antibodies and detecting specific binding to the analyte (e.g., UCH-L1 and/or GFAP). For example, the antibody, or antibody fragment thereof, may specifically bind to the analyte (e.g., UCH-L1 and/or GFAP). If desired, one or more of the antibodies can be used in combination with one or more commercially available monoclonal/polyclonal antibodies. Such antibodies are available from companies such as R&D Systems, Inc. (Minneapolis, MN) and Enzo Life Sciences International, Inc. (Plymouth Meeting, PA).

The presence or amount of analyte (e.g., UCH-L1 and/or GFAP) present in a body sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, MN)). An example of a point-of-care device that can be used is i-STAT (Abbott, Laboratories, Abbott Park, Ill.). Other methods that can be used include a chemiluminescent microparticle immunoassay, in particular one employing the ARCHITECT or Alinity automated analyzers (Abbott Laboratories, Abbott Park, Ill.), as examples. Other methods include, for example, mass spectrometry, and immunohistochemistry (e.g., with sections from tissue biopsies), using anti-analyte (e.g., anti-UCH-L1 and/or anti-GFAP) antibodies (monoclonal, polyclonal, chimeric, humanized, human, etc.) or antibody fragments thereof against analyte (e.g., UCH-L1 and/or GFAP). Other methods of detection include those described in, for example, U.S. Pat. Nos. 6,143,576; 6, 113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte (e.g., UCH-L1 and/or GFAP) can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

A homogeneous format may be used. For example, after the test sample is obtained from a subject, a mixture is prepared. The mixture contains the test sample being assessed for analyte (e.g., UCH-L1 and/or GFAP), a first specific binding partner, and a second specific binding partner. The order in which the test sample, the first specific binding partner, and the second specific binding partner are added to form the mixture is not critical. The test sample is simultaneously contacted with the first specific binding partner and the second specific binding partner. In some embodiments, the first specific binding partner and any UCH-L1 and/or GFAP contained in the test sample may form a first specific binding partner-analyte (e.g., UCH-L1 and/or GFAP)-antigen complex and the second specific binding partner may form a first specific binding partner-analyte of interest (e.g., UCH-L1 and/or GFAP)-second specific binding partner complex. In some embodiments, the second specific binding partner and any UCH-L1 and/or GFAP contained in the test sample may form a second specific binding partner-analyte (e.g., UCH-L1)-antigen complex and the first specific binding partner may form a first specific binding partner-analyte of interest (e.g., UCH-L1 and/or GFAP)-second specific binding partner complex. The first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

A heterogeneous format may be used. For example, after the test sample is obtained from a subject, a first mixture is prepared. The mixture contains the test sample being assessed for analyte (e.g., UCH-L1 and/or GFAP) and a first specific binding partner, wherein the first specific binding partner and any UCH-L1 and/or GFAP contained in the test sample form a first specific binding partner-analyte (e.g., UCH-L1 and/or GFAP)-antigen complex. The first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The order in which the test sample and the first specific binding partner are added to form the mixture is not critical.

The first specific binding partner may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fc. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

After the mixture containing the first specific binding partner-analyte (e.g., UCH-L1 or GFAP) antigen complex is formed, any unbound analyte (e.g., UCH-L1 and/or GFAP) is removed from the complex using any technique known in the art. For example, the unbound analyte (e.g., UCH-L1 and/or GFAP) can be removed by washing. Desirably, however, the first specific binding partner is present in excess of any analyte (e.g., UCH-L1 and/or GFAP) present in the test sample, such that all analyte (e.g., UCH-L1 and/or GFAP) that is present in the test sample is bound by the first specific binding partner.

After any unbound analyte (e.g., UCH-L1 and/or GFAP) is removed, a second specific binding partner is added to the mixture to form a first specific binding partner-analyte of interest (e.g., UCH-L1 and/or GFAP)-second specific binding partner complex. The second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

The use of immobilized antibodies or antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

In some aspects, it is possible that other antibodies can be selected which similarly may assist with maintaining the dynamic range and low end sensitivity of the immunoassays. For example, it may be useful to select at least one first antibody (such as a capture antibody or first specific binding partner) that binds to an epitope near the N-terminus of the 38 kDa BDP and at least one second antibody (such as a detection antibody or second specific binding partner) that binds to an epitope near the middle of the 38 kDa BDP, e.g., near the middle of the 38 kDa BDP, and that does not overlap with the first antibody. Other variations are possible and could be readily tested by one of ordinary skill, such as by confirming antibodies bind to different epitopes by examining binding to short peptides, and then screening antibody pairs using low calibrator concentration. Moreover, selecting antibodies of differing affinity for GFAP also can assist with maintaining or increasing the dynamic range of the assay. GFAP antibodies have been described in the literature and are commercially available.

### (1) Sandwich Immunoassay

A sandwich immunoassay measures the amount of antigen between two layers of antibodies (i.e., at least one capture antibody) and a detection antibody (i.e., at least one detection antibody). The capture antibody and the detection antibody bind to different epitopes on the antigen, e.g., analyte of interest such as UCH-L1 and/or GFAP. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte (e.g., UCH-L1 and/or GFAP) in a test sample. More specifically, the at least two antibodies bind to certain epitopes of analyte (e.g., UCH-L1 and/or GFAP) forming an immune complex which is referred to as a “sandwich”. One or more antibodies can be used to capture the analyte (e.g., UCH-L1 and/or GFAP) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a test sample suspected of containing analyte (e.g., UCH-L1 and/or GFAP) do not bind to all or part of an epitope recognized by the second or subsequent antibodies, thereby interfering with the ability of the one or more second detection antibodies to bind to the analyte (e.g., UCH-L1 and/or GFAP).

The antibodies may be used as a first antibody in said immunoassay. The antibody immunospecifically binds to epitopes on analyte (e.g., UCH-L1 and/or GFAP). In addition to the antibodies of the present disclosure, said immunoassay may comprise a second antibody that immunospecifically binds to epitopes that are not recognized or bound by the first antibody.

A test sample suspected of containing analyte (e.g., UCH-L1 and/or GFAP) can be contacted with at least one first capture antibody (or antibodies) and at least one second detection antibodies either simultaneously or sequentially. In the sandwich assay format, a test sample suspected of containing analyte (e.g., UCH-L1 and/or GFAP) is first brought into contact with the at least one first capture antibody that specifically binds to a particular epitope under conditions which allow the formation of a first antibody-analyte (e.g., UCH-L1 and/or GFAP) antigen complex. If more than one capture antibody is used, a first multiple capture antibody-UCH-L1 and/or GFAP antigen complex is formed. In a sandwich assay, the antibodies, preferably, the at least one capture antibody, are used in molar excess amounts of the maximum amount of analyte (e.g., UCH-L1 and/or GFAP) expected in the test sample. For example, from about 5 μg/mL to about 1 mg/mL of antibody per ml of microparticle coating buffer may be used.

i. Anti-UCH-L1 Capture Antibody

Optionally, prior to contacting the test sample with the at least one first capture antibody, the at least one first capture antibody can be bound to a solid support which facilitates the separation the first antibody-analyte (e.g., UCH-L1 and/or GFAP) complex from the test sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte (e.g., UCH-L1 and/or GFAP). Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl) carbodiimide.

After the test sample suspected of containing analyte (e.g., UCH-L1 and/or GFAP) is incubated in order to allow for the formation of a first capture antibody (or multiple antibody)-analyte (e.g., UCH-L1 and/or GFAP) complex. The incubation can be carried out at a pH of from about 4.5 to about 10.0, at a temperature of from about 2° C. to about 45° C., and for a period from at least about one (1) minute to about eighteen (18) hours, from about 2-6 minutes, from about 7-12 minutes, from about 5-15 minutes, or from about 3-4 minutes.

ii. Detection Antibody

After formation of the first/multiple capture antibody-analyte (e.g., UCH-L1 and/or GFAP) complex, the complex is then contacted with at least one second detection antibody (under conditions that allow for the formation of a first/multiple antibody-analyte (e.g., UCH-L1 and/or GFAP) antigen-second antibody complex). In some embodiments, the test sample is contacted with the detection antibody simultaneously with the capture antibody. If the first antibody-analyte (e.g., UCH-L1 and/or GFAP) complex is contacted with more than one detection antibody, then a first/multiple capture antibody-analyte (e.g., UCH-L1 and/or GFAP)-multiple antibody detection complex is formed. As with first antibody, when the at least second (and subsequent) antibody is brought into contact with the first antibody-analyte (e.g., UCH-L1 and/or GFAP) complex, a period of incubation under conditions similar to those described above is required for the formation of the first/multiple antibody-analyte (e.g., UCH-L1 and/or GFAP)-second/multiple antibody complex. Preferably, at least one second antibody contains a detectable label. The detectable label can be bound to the at least one second antibody prior to, simultaneously with or after the formation of the first/multiple antibody-analyte (e.g., UCH-L1 and/or GFAP)-second/multiple antibody complex. Any detectable label known in the art can be used.

Chemiluminescent assays can be performed in accordance with the methods described in Adamczyk et al., Anal. Chim. Acta 579 (1): 61-67 (2006). While any suitable assay format can be used, a microplate chemiluminometer (Mithras LB-940, Berthold Technologies U.S.A., LLC, Oak Ridge, TN) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-antigen (e.g., UCH-L1 and/or GFAP) complexes form. Alternatively, if a second specific binding partner is used and the second specific binding partner is detectably labeled with an acridinium compound, detectably labeled first specific binding partner-analyte (e.g., UCH-L1 and/or GFAP)-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte (e.g., UCH-L1 and/or GFAP) is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest (e.g., UCH-L1 and/or GFAP) in the sample can be quantified. Specifically, the amount of analyte (e.g., UCH-L1 and/or GFAP) in the sample is proportional to the intensity of the signal generated. The amount of analyte (e.g., UCH-L1 and/or GFAP) present can be quantified by comparing the amount of light generated to a standard curve for analyte (e.g., UCH-L1 and/or GFAP) or by comparison to a reference standard. The standard curve can be generated using serial dilutions or solutions of known concentrations of analyte (e.g., UCH-L1 and/or GFAP) by mass spectroscopy, gravimetric methods, and other techniques known in the art.

### (2) Forward Competitive Inhibition Assay

In a forward competitive format, an aliquot of labeled analyte of interest (e.g., analyte (e.g., UCH-L1 and/or GFAP) having a fluorescent label, a tag attached with a cleavable linker, etc.) of a known concentration is used to compete with analyte of interest (e.g., UCH-L1 and/or GFAP) in a test sample for binding to analyte of interest antibody (e.g., UCH-L1 and/or GFAP antibody).

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above.

### (3) Reverse Competitive Inhibition Assay

In a reverse competition assay, an immobilized analyte of interest (e.g., UCH-L1 and/or GFAP) can either be sequentially or simultaneously contacted with a test sample and at least one labeled antibody.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The concentration of analyte of interest in the test sample can then be determined by comparing the quantity of detectable label as described above.

### (4) One-Step Immunoassay or “Capture on the Fly” Assay

In a capture on the fly immunoassay, a solid substrate is pre-coated with an immobilization agent. The capture agent, the analyte (e.g., UCH-L1 and/or GFAP) and the detection agent are added to the solid substrate together, followed by a wash step prior to detection. The capture agent can bind the analyte (e.g., UCH-L1 and/or GFAP) and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest (e.g., UCH-L1 and/or GFAP). The second specific binding member comprises a detectable label and binds to an analyte of interest (e.g., UCH-L1 and/or GFAP). The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or concentrations, measured, determined or assessed.

The use of a capture on the fly assay can be done in a variety of formats as described herein, and known in the art. For example, the format can be a sandwich assay such as described above, but alternately can be a competition assay, can employ a single specific binding member, or use other variations such as are known.

### 10. Other Factors

The methods of diagnosing, prognosticating, and/or assessing, as described above, can further include using other factors for the diagnosis, prognostication, and assessment. In some embodiments, an acquired brain injury such as a traumatic brain injury, may be diagnosed using the Glasgow Coma Scale or the Extended Glasgow Outcome Scale (GOSE). Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate. Another example is the Rivermead Post-Concussion Symptoms Questionairre, a self-report scale to measure the severity of post-concussive symptoms following TBI. Patients are asked to rate how severe each of 16 symptoms (e.g., headache, dizziness, nausea, vomiting) has been over the past 24 hours. In each case, the symptom is compared with how severe it was before the injury occurred (premorbid). These symptoms are reported by severity on a scale from 0 to 4: not experienced, no more of a problem, mild problem, moderate problem, and severe problem.

### 11. Samples

In some embodiments, the sample is obtained after the subject, such as a human subject, sustained an injury to the head caused by physical shaking, blunt impact by an external mechanical or other force that results in a closed or open head trauma, one or more falls, explosions or blasts or other types of blunt force trauma. In some embodiments, the sample is obtained after the subject, such as a human subject, has ingested or been exposed to a fire, chemical, toxin or combination of a fire, chemical and toxin. Examples of such chemicals and/or toxins include, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a subject, such as a human subject, that suffers from an autoimmune disease, a metabolic disorder, a brain tumor, hypoxia, a viral infection (e.g., SARS-COV-2), a fungal infection, a bacterial infection, meningitis, hydrocephalus, or any combinations thereof.

In yet another embodiment, the methods described herein use samples that also can be used to determine whether or not a subject has or is at risk of developing a TBI (such as a mild TBI, moderate TBI, severe TBI, or moderate-to-severe TBI) by determining the levels of UCH-L1 and/or GFAP in a subject using the anti-UCH-L1 and/or anti-GFAP antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, a viral infection (e.g., SARS-COV-2), a fungal infection, a bacterial infection, meningitis, hydrocephalus, or any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, a viral infection (e.g., SARS-COV-2), a fungal infection, a bacterial infection, meningitis, hydrocephalus, or any combinations thereof), and/or who demonstrates an unfavorable (i.e., clinically undesirable) concentration or amount of UCH-L1 and/or GFAP or UCH-L1 and/or GFAP fragment, as described herein.

a. Test or Biological Sample

As used herein, “sample”, “test sample”, “biological sample” refer to fluid sample containing or suspected of containing UCH-L1 and/or GFAP. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing UCH-L1 and/or GFAP may be assayed directly. In a particular example, the source of UCH-L1 and/or GFAP is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, oropharyngeal specimen, nasopharyngeal specimen, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 uL, about 0.1 μL, about 1 uL, about 5 uL, about 10 uL, about 100 uL, about 1 mL, about 5 mL, about 10 mL, or the like. In some cases, the volume of the fluid sample is between about 0.01 uL and about 10 mL, between about 0.01 uL and about 1 mL, between about 0.01 uL and about 100 uL, or between about 0.1 uL and about 10 uL.

In some cases, the fluid sample may be diluted prior to use in an assay. For example, in embodiments where the source of UCH-L1 and/or GFAP is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source of UCH-L1 and/or GFAP is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

b. Controls

It may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e., fluorescent signal intensity, if a fluorescent label is used. Using samples taken from multiple donors, standard curves can be provided for reference levels of the UCH-L1 and/or GFAP in normal healthy tissue, as well as for “at-risk” levels of the UCH-L1 and/or GFAP in tissue taken from donors, who may have one or more of the characteristics set forth above.

Thus, in view of the above, a method for determining the presence, amount, or concentration of UCH-L1 and/or GFAP in a test sample is provided. The method comprises assaying the test sample for UCH-L1 and/or GFAP by an immunoassay, for example, employing at least one capture antibody that binds to an epitope on UCH-L1 and/or GFAP and at least one detection antibody that binds to an epitope on UCH-L1 and/or GFAP which is different from the epitope for the capture antibody and optionally includes a detectable label, and comprising comparing a signal generated by the detectable label as a direct or indirect indication of the presence, amount or concentration of UCH-L1 and/or GFAP in the test sample to a signal generated as a direct or indirect indication of the presence, amount or concentration of UCH-L1 and/or GFAP in a calibrator. The calibrator is optionally, and is preferably, part of a series of calibrators in which each of the calibrators differs from the other calibrators in the series by the concentration of UCH-L1 and/or GFAP.

### 12. Kit

Provided herein is a kit, which may be used for assaying or assessing a test sample for UCH-L1 and/or GFAP or UCH-L1 and/or GFAP fragment. The kit comprises at least one component for assaying the test sample for UCH-L1 and/or GFAP instructions for assaying the test sample for UCH-L1 and/or GFAP. For example, the kit can comprise instructions for assaying the test sample for UCH-L1 and/or GFAP by immunoassay, e.g., chemiluminescent microparticle immunoassay. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component may include at least one composition comprising one or more isolated antibodies or antibody fragments thereof that specifically bind to UCH-L1 and/or GFAP. The antibody may be a UCH-L1 and/or GFAP capture antibody and/or a UCH-L1 and/or GFAP detection antibody.

Alternatively or additionally, the kit can comprise a calibrator or control, e.g., purified, and optionally lyophilized, UCH-L1 and/or GFAP, and/or at least one container (e.g., tube, microtiter plates or strips, which can be already coated with an anti-UCH-L1 and/or GFAP monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying UCH-L1 and/or GFAP. The reference standards may be employed to establish standard curves for interpolation and/or extrapolation of UCH-L1 and/or GFAP concentrations. The reference standards may include a high UCH-L1 and/or GFAP concentration level, for example, about 100000 pg/mL, about 125000 pg/mL, about 150000 pg/mL, about 175000 pg/mL, about 200000 pg/mL, about 225000 pg/mL, about 250000 pg/mL, about 275000 pg/mL, or about 300000 pg/mL; a medium UCH-L1 and/or GFAP concentration level, for example, about 25000 pg/mL, about 40000 pg/mL, about 45000 pg/mL, about 50000 pg/mL, about 55000 pg/mL, about 60000 pg/mL, about 75000 pg/mL or about 100000 pg/mL; and/or a low UCH-L1 and/or GFAP concentration level, for example, about 1 pg/mL, about 5 pg/mL, about 10 pg/mL, about 12.5 pg/mL, about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, or about 100 pg/mL.

Any antibodies, which are provided in the kit, such as recombinant antibodies specific for UCH-L1 and/or GFAP, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., UCH-L1 and/or GFAP) or reagents for detecting the analyte (e.g., UCH-L1 and/or GFAP). The antibodies, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

a. Adaptation of Kit and Method

The kit (or components thereof), as well as the method for assessing or determining the concentration of UCH-L1 and/or GFAP in a test sample by an immunoassay as described herein, can be adapted for use in a variety of automated and semi-automated systems (including those wherein the solid phase comprises a microparticle), as described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, Ill.) as Abbott Point of Care (i-STAT or i-STAT Alinity, Abbott Laboratories) as well as those described in U.S. Pat. Nos. 5,089,424 and 5,006,309, and as commercially marketed, e.g., by Abbott Laboratories (Abbott Park, Ill.) as ARCHITECT or the series of Abbott Alinity devices.

Some of the differences between an automated or semi-automated system as compared to a non-automated system (e.g., ELISA) include the substrate to which the first specific binding partner (e.g., analyte antibody or capture antibody) is attached (which can affect sandwich formation and analyte reactivity), and the length and timing of the capture, detection, and/or any optional wash steps. Whereas a non-automated format such as an ELISA may require a relatively longer incubation time with sample and capture reagent (e.g., about 2 hours), an automated or semi-automated format (e.g., ARCHITECT and any successor platform, Abbott Laboratories) may have a relatively shorter incubation time (e.g., approximately 18 minutes for ARCHITECT). Similarly, whereas a non-automated format such as an ELISA may incubate a detection antibody such as the conjugate reagent for a relatively longer incubation time (e.g., about 2 hours), an automated or semi-automated format (e.g., ARCHITECT and any successor platform) may have a relatively shorter incubation time (e.g., approximately 4 minutes for the ARCHITECT and any successor platform).

Other platforms available from Abbott Laboratories include, but are not limited to, AxSYM, IMx (see, e.g., U.S. Pat. No. 5,294,404, which is hereby incorporated by reference in its entirety), PRISM, EIA (bead), and Quantum II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT, Abbott Laboratories) electrochemical immunoassay system that performs sandwich immunoassays. Immunosensors and their methods of manufacture and operation in single-use test devices are described, for example in, U.S. Pat. No. 5,063,081, U.S. Patent App. Publication Nos. 2003/0170881, 2004/0018577, 2005/0054078, and 2006/0160164, which are incorporated in their entireties by reference for their teachings regarding same.

Regarding the adaptation of an assay to the i-STAT system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an i-STAT cartridge with a fluidics format suitable for immunoassay. On a portion of the silicon chip, there is a specific binding partner for UCH-L1 and/or GFAP, such as one or more UCH-L1 and/or GFAP antibodies (one or more monoclonal/polyclonal antibody or a fragment thereof, a variant thereof, or a fragment of a variant thereof that can bind UCH-L1 and/or GFAP) or one or more anti-UCH-L1 and/or GFAP DVD-Igs (or a fragment thereof, a variant thereof, or a fragment of a variant thereof that can bind UCH-L1 and/or GFAP), either of which can be detectably labeled. Within the fluid pouch of the cartridge is an aqueous reagent that includes p-aminophenol phosphate.

In operation, a sample from a subject suspected of suffering from TBI is added to the holding chamber of the test cartridge, and the cartridge is inserted into the i-STAT reader. A pump element within the cartridge pushes the sample into a conduit containing the chip. The sample is brought into contact with the sensors allowing the enzyme conjugate to dissolve into the sample. The sample is oscillated across the sensors to promote formation of the sandwich of approximately 2-12 minutes. In the penultimate step of the assay, the sample is pushed into a waste chamber and wash fluid, containing a substrate for the alkaline phosphatase enzyme, is used to wash excess enzyme conjugate and sample off the sensor chip. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of UCH-L1 and/or GFAP in the sample by means of an embedded algorithm and factory-determined calibration curve.

The automated and semi-automated systems described herein for use in the methods of the present disclosure can utilize one or more computer programs, software or algorithms to provide the determination (readout) of whether to perform an CT scan (e.g., based on a positive result) or not to perform an CT scan (e.g., based on a negative result). For example, the computer program(s) or software (e.g., making use of an algorithm) can provide an interpretation (regardless of whether one, two or more samples are used) that: (1) when the level of GFAP and UCH-L1 is less than the reference level (or cutoff) that the result is negative meaning that no CT scan will be performed; or (2) when the level or GFAP and/or UCH-L1 is greater than or equal to the reference level (or cutoff) that the result is positive meaning that an CT scan will be performed. The computer program(s) or software can provide other appropriate interpretations, such as, whether the reference level is or is not correlated with a positive head CT, the presence of an intracranial lesion or with control subjects that have not suffered a traumatic brain injury, whether the subject suffering from the TBI should be monitored and/or treated with a TBI treatment, etc. Such computer programs or software are well known in the art.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT conjugate diluent employed in certain kits (Abbott Laboratories, Abbott Park, Ill.) and containing 2-(N-morpholino) ethanesulfonic acid (MES), a salt, a protein blocker, an antimicrobial agent, and a detergent. An exemplary calibrator diluent is ARCHITECT human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an i-STAT cartridge format, using a nucleic acid sequence linked to the signal antibody as a signal amplifier.

While certain embodiments herein are advantageous when employed to assess disease, such as traumatic brain injury, the assays and kits also optionally can be employed to assess UCH-L1 and/or GFAP in other diseases, disorders, and conditions as appropriate.

The method of assay also can be used to identify a compound that ameliorates diseases, such as traumatic brain injury. For example, a cell that expresses UCH-L1 and/or GFAP can be contacted with a candidate compound. The level of expression of UCH-L1 and/or GFAP in the cell contacted with the compound can be compared to that in a control cell using the method of assay described herein.

The present disclosure has multiple aspects, illustrated by the following non-limiting examples.

### Example 1

### Alinity-i UCH-L1 Assay

A core laboratory (e.g., ARCHITECT or Alinity) UCH-L1 assay was used in a TBI patient population study. The assay is a chemiluminescent microparticle immunoassay (CMIA) used for the quantitative determination of UCH-L1 in human plasma and serum on the ARCHITECT or Alinity i systems. Monoclonal antibody pairs, such as pairings of Antibody A, B and C, used as capture agent or detection agent, were used. Antibody A is an exemplary anti-UCH-L1 antibody that was internally developed at Abbott Laboratories (Abbott Park, Ill.). Antibody B and C recognize different epitopes of UCH-L1 and enhance the detection of antigen in the sample and were developed by Banyan Biomarkers (Alachua, Florida). Other antibodies that were internally developed at Abbott Laboratories (Abbott Park, Ill.) or are publicly available also show or are expected to show similar enhancement of signal when used together as capture antibodies or detection antibodies, in various combinations. The UCH-L1 assay design was evaluated against key performance attributes.

### Example 2

### Alinity-i® GFAP Assay

A core laboratory (e.g., ARCHITECT or Alinity) GFAP assay was used in a TBI patient population study. The assay is a chemiluminescent microparticle immunoassay (CMIA) used for the quantitative determination of GFAP in human plasma and serum on the ARCHITECT or Alinity i systems. Monoclonal antibody pairs, such as Antibody A as a capture monoclonal antibody and Antibody B as a detection monoclonal antibody, were used. Antibody A and Antibody B are exemplary anti-GFAP antibodies that were internally developed at Abbott Laboratories (Abbott Park, Ill.). The GFAP assay design was evaluated against key performance attributes.

### Example 3

### TBI Population Study (TRACK-TBI)

The Transforming Research and Clinical Knowledge in Traumatic Brain Injury (TRACK-TBI) study is a large and complex project. Its institutional and public-private partnership is comprised of over 11 clinical sites, 7 Cores, for a total of nearly 50 collaborating institutions, corporations, and philanthropy. An earlier TRACK-TBI Pilot study, based on clinical data from three clinical sites, helped refine TBI Common Data Elements and created a prototype of the TBI Information Commons for the TRACK-TBI study.

Subject Groups: A total of 2,700 to 3000 TBI patients were enrolled evenly across 3 clinical groups, differentiated by clinical care path: 1. Patients evaluated in the Emergency Department and discharged (ED); 2. Patients admitted to the hospital, but not to ICU (ADM); and 3. Patients admitted to the ICU (ICU). An additional 100 patients per clinical group (approximately 300) with extracranial trauma but no TBI were enrolled as controls for a total enrollment of approximately 3000 patients. This stratification plan facilitated comparative effectiveness research (CER) analysis and was not constrained by traditional differentiation into “Mild/Moderate/Severe” TBI. Data collection was dependent on the clinical care path (ED, ADM, ICU) and requirements of each aim. Patients in each group were stratified into 3 cohorts that define the extent of data to be collected.

The controls were healthy patients who had not reported a TBI within the last twelve (12) months and did not suffer from any type of a medically diagnosed neurological disease (e.g., multiple sclerosis, Alzheimer's disease, ALS, etc.).

Subject Eligibility: Adult patients were enrolled of all ages presenting to the Emergency Department (ED) with a history of acute TBI as per American Congress of Rehabilitation Medicine (ACRM) Criteria, in which the patient had sustained a traumatically induced physiological disruption of brain function, as manifested by ≥one of the following: any period of loss of consciousness (LOC); any loss of memory for events (e.g., amnesia) immediately before or after the accident; any alteration of mental state at the time of the accident (feeling dazed, disoriented, and/or confused); and/or focal neurologic deficits that may or may not be permanent. Traumatically induced included the head being struck, the head striking an object, or the brain undergoing an acceleration/deceleration movement (e.g., whiplash) without direct external trauma to the head.

The Inclusion/Exclusion Criteria used is shown in Table A.

TABLE A

| | | Data | |
|---|---|---|---|
| Criterion | | Source | Comments |
| Inclusion Criteria | | | |
| 1. | Age 0-100 | Chart | |
| 2. | Documented/verified TBI (ACRM | Chart, | |
| | Criteria) | Interview | |
| 3. | Injury occurred <24 hours ago | Chart, | |
| | | Interview | |
| 4. | Acute brain CT for clinical care | Chart | Subject must have brain CT scan |
| 5. | Visual acuity/hearing adequate for | Chart, | |
| | testing | Interview | |
| 6. | Fluency in English or Spanish | Chart, | Test battery or personnel availability |
| | | Interview | |
| 7. | Ability to provide informed consent | Interview | |
| Exclusion Criteria | | | |
| 1. | Significant polytrauma that would | Chart | Significant body trauma may confound |
| | interfere with follow-up and outcome | | TBI outcomes testing. |
| | assessment | | |
| 2. | Prisoners or patients in custody | Chart, | |
| | | Interview | |
| 3. | Pregnancy in female subjects | Chart, | |
| | | Interview | |
| 4. | Patients on psychiatric hold | Chart | |
| 5. | Major debilitating baseline mental | Chart, | Debilitating psychiatric disorders can |
| | health disorders (e.g., schizophrenia | Interview | significantly impact the reliability of |
| | or bipolar disorder) that would | | follow up and/or pose difficulties in |
| | interfere with follow-up and the | | attributing to index TBI. |
| | validity of outcome assessment | | |
| 6. | Major debilitating neurological | Chart, | Documented debilitating baseline |
| | disease (e.g., stroke, CVA, dementia, | Interview | cognitive impairment will confound |
| | tumor) impairing baseline awareness | | outcome assessment in addition to not |
| | cognition or validity of follow-up and | | being fully consentable. |
| | outcome assessment | | |
| 7. | Significant history of pre-existing | Chart, | |
| | conditions that would interfere with | Interview | |
| | follow-up and outcome assessment | | |
| | (e.g., substance abuse, alcoholism, | | |
| | HIV/AIDS, major transmittable | | |
| | diseases that may interfere with | | |
| | consent, end-stage cancers, learning | | |
| | disabilities, developmental disorders) | | |
| 8. | Low likelihood of follow-up (e.g., | Interview | |
| | participant or family indicating low | | |
| | interest, residence in another state or | | |
| | country, homelessness or lack of | | |
| | reliable contacts) | | |
| 9. | Current participant in an | Chart, | Exception to co-enrollment exclusion is |
| | interventional trial (e.g., drug, device, | Interview | made for sites participating in |
| | behavioral) | | Resuscitation Outcomes Consortium |
| | | | Prehospital Tranexamic Acid for TBI |
| | | | Study. |
| 10. | Penetrating TBI | Chart | |
| 11. | Spinal cord injury with ASIA score | Chart | |
| | of C or worse | | |

UCH-L1 and GFAP were measured in a sample size of 276 TRACK TBI patients in the core laboratory (i.e., ARCHITECT or Alinity) format (Table B). The assays performed to measure the UCH-L1 and GFAP were performed individually, optimized and then the measurements for each of UCH-L1 and GFAP were combined.

TABLE B
Subject Characteristics

| | | | Chi Square |
|---|---|---|---|
| Subject Characteristics | n (%) | | P-value |
| Sex (N = 276) | | | <0.0001 |
| Male | 200 | (72.5%) | |
| Female | 76 | (27.5%) | |
| GCS (N = 269) | | | 0.9514 |
| Mild (13-15) | 134 | (49.8%) | |
| Moderate/Severe (3-12) | 135 | (50.2%) | |
| CT (N = 274) | | | <0.0001 |
| Negative | 80 | (29.2%) | |
| Positive | 194 | (70.8%) | |
| GOSE (N = 254) | | | 0.0447 |
| GOSE ≤3 | 143 | (56.3%) | |
| GOSE ≥4 | 111 | (43.7%) | |
| Loss of Consciousness (N = 253) | | | <0.0001 |
| Yes | 223 | (82.0%) | |
| No | 16 | (5.9%) | |
| Suspected | 14 | (5.2%) | |
| Unknown | 19 | (7.0%) | |
| Cause of Injury (N = 273) | | | <0.0001 |
| Road Traffic Incident | 173 | (63.4%) | |
| Incidental Fall | 67 | (24.5%) | |
| Other Non-intentional Injury | 8 | (2.9%) | |
| Violence/Assault | 19 | (7.0%) | |
| Other | 6 | (2.2%) | |
| | Median [min, max] | | |
| Age (N = 276) | 41 | [18, 88] | |
| Days from Injury to Blood Draw | 16.9 | [5.0, 24.4] | |

GFAP levels were correlated with Glasgow Coma Scale (GCS) score severity (mild [GCS 13-15] versus moderate/severe [GCS 3-12]), GOSE (0-3 versus 4-8), and positive and negative CT scans for TBI. Each of the GCS, GOSE and CT scan conditions were dichotomized, allowing calculation of ROC curves and associated cutoffs. Samples were assessed within approximately one week, two weeks, or three weeks from injury and the results, together with the reference levels (e.g., cutoffs) are as shown below in Table C.

Additionally, UCH-L1 levels were correlated with Glasgow Coma Scale (GCS) score severity (mild [GCS 13-15] versus moderate/severe [GCS 3-12]), GOSE (0-3 versus 4-8), and positive and negative CT scans for TBI. Samples were assessed within approximately one week, two weeks, or three weeks from injury and the results, together with the reference levels (e.g., cutoffs) are as shown below in Table C. As mentioned previously, the assays for GFAP and UCH-L1 were performed individually, optimized and then the measurements for each of UCH-L1 and GFAP were combined to provide the results in Table C.

The reference levels shown in Table C can be used when determining the concentration of GFAP alone, UCH-L1 alone, or both GFAP and UCH-L1.

TABLE C

| | | | Lower | Upper | Number |
|---|---|---|---|---|---|
| | | | Cutoff | Cutoff | of |
| Biomarker | Measure | Time | Range | Range | Patients |
| GFAP | GCS | 1 Week | 17 | 425 | 90 |
| GFAP | GCS | 2 Weeks | 17 | 431 | 117 |
| GFAP | GCS | 3 Weeks | 17 | 415 | 62 |
| GFAP | GOSE | 1 Week | 17 | 431 | 88 |
| GFAP | GOSE | 2 Weeks | 17 | 375 | 109 |
| GFAP | GOSE | 3 Weeks | 17 | 431 | 57 |
| GFAP | CT | 1 Week | 17 | 307 | 91 |
| GFAP | CT | 2 Weeks | 18 | 294 | 120 |
| GFAP | CT | 3 Weeks | 16 | 291 | 63 |
| UCH-L1 | GCS | 1 Week | 56 | 267 | 90 |
| UCH-L1 | GCS | 2 Weeks | 61 | 248 | 117 |
| UCH-L1 | GCS | 3 Weeks | 56 | 250 | 62 |
| UCH-L1 | GOSE | 1 Week | 49 | 275 | 88 |
| UCH-L1 | GOSE | 2 Weeks | 63 | 267 | 109 |
| UCH-L1 | GOSE | 3 Weeks | 53 | 275 | 57 |
| UCH-L1 | CT | 1 Week | 55 | 250 | 91 |
| UCH-L1 | CT | 2 Weeks | 51 | 248 | 120 |
| UCH-L1 | CT | 3 Weeks | 55 | 243 | 63 |

Other exemplary reference levels (e.g., cutoffs) that can be used are shown below in Tables D-H. The reference levels shown in Tables D-H can be used when determining the concentration of GFAP alone. UCH-L1 alone, or both GFAP and UCH-L1.

TABLE D
GFAP and UCH-L1

| | Determining that a | GFAP level | UCH-L1 level | |
|---|---|---|---|---|
| Option | subject: | (pg/mL) | (pg/mL) | Measure |
| 1 | Has not suffered or | Less than about | Less than about | Glasgow Coma Scale |
| | recovered from an actual | 18 pg/mL | 63 pg/mL | Score; Extended |
| | ABI | | | Glasgow Outcome |
| | | | | Scale Score, a positive |
| | | | | CT scan for a TBI, a |
| | | | | negative scan for a |
| | | | | TBI or a combination |
| | | | | thereof |
| | Should be monitored for | Between about | Between about | Glasgow Coma Scale |
| | recovery from an actual ABI | 18 pg/mL at the | 63 pg/mL at the | Score; Extended |
| | | lower limit up to | lower limit to | Glasgow Outcome |
| | | about 431 | about 275 | Scale Score, a positive |
| | | pg/mL at the | pg/mL at the | CT scan for a TBI, a |
| | | upper limit | upper limit | negative scan for a |
| | | | | TBI or a combination |
| | | | | thereof |
| | Sustained and is not | Greater than | Greater than | Glasgow Coma Scale |
| | recovering from an actual | about 431 | about 275 | Score; Extended |
| | ABI | pg/mL | pg/mL | Glasgow Outcome |
| | | | | Scale Score, a positive |
| | | | | CT scan for a TBI, a |
| | | | | negative scan for a |
| | | | | TBI or a combination |
| | | | | thereof |
| 2 | Has not suffered or | Less than about | Less than about | A negative head CT |
| | recovered from an actual | 18 pg/mL | 55 pg/mL | scan |
| | ABI | | | |
| | Should be monitored for | Between about | Between about | A positive head CT |
| | recovery from an actual ABI | 18 pg/mL to | 55 pg/mL to | scan or a negative |
| | | about 307 | about 250 | head CT scan |
| | | pg/mL | pg/mL | |
| | Sustained and is not | Greater than | Greater than | A positive head CT |
| | recovering from an actual | about 307 | about 250 | scan |
| | ABI | pg/mL | pg/mL | |
| 3 | Has not suffered or | Less than about | Less than about | GCS score severity for |
| | recovered from an actual | 17 pg/mL | 61 pg/mL | a mild TBI (GCS 13- |
| | ABI | | | 15) |
| | Should be monitored for | Between a range | Between a range | GCS score severity for |
| | recovery from an actual ABI | of about 17 | of about 61 | a moderate, severe, or |
| | | pg/mL to about | pg/mL to about | a moderate-to-severe |
| | | 431 pg/mL | 267 pg/mL | TBI (GCS 3-12) |
| | Sustained and is not | Greater than | Greater than | GCS score severity for |
| | recovering from an actual | about 431 | about 267 | a moderate, severe, or |
| | ABI | pg/mL | pg/mL | a moderate-to-severe |
| | | | | TBI (GCS 3-12) |
| 4 | Has not suffered or | Less than about | Less than about | GOSE score of 4-8 |
| | recovered from an actual | 17 pg/mL | 63 pg/mL | |
| | ABI | | | |
| | Should be monitored for | Between about | Between about | GOSE score of 0-3 |
| | recovery from an actual ABI | 17 pg/mL to | 63 pg/mL to | |
| | | about 431 | about 275 | |
| | | pg/mL | pg/mL | |
| | Sustained and is not | Greater than | Greater than | GOSE score of 0-3 |
| | recovering from an actual | about 431 | about 275 | |
| | ABI | pg/mL | pg/mL | |

TABLE E
GFAP

| Option | Determining that a subject: | GFAP level (pg/mL) |
|---|---|---|
| 1 | Has not suffered or recovered from an actual ABI | Less than about 16 |
| | Should be monitored for recovery from an actual ABI | Between a range of about 16 to |
| | | about 291 |
| | Sustained and is not recovering from an actual ABI | Greater than about 291 |
| 2 | Has not suffered or recovered from an actual ABI | Less than about 16 |
| | Should be monitored for recovery from an actual ABI | Between a range of about 16 to |
| | | about 294 |
| | Sustained and is not recovering from an actual ABI | Greater than about 294 |
| | Has not suffered or recovered from an actual ABI | Less than about 16 |
| 3 | Should be monitored for recovery from an actual ABI | Between a range of about 16 to |
| | | about 307 |
| | Sustained and is not recovering from an actual ABI | Greater than about 307 |
| 4 | Has not suffered or recovered from an actual ABI | Less than about 16 |
| | Should be monitored for recovery from an actual ABI | Between a range of about 16 to |
| | | about 375 |
| | Sustained and is not recovering from an actual ABI | Greater than about 375 |
| 5 | Has not suffered or recovered from an actual ABI | Less than about 16 |
| | Should be monitored for recovery from an actual ABI | Between a range of about 16 to |
| | | about 415 |
| | Sustained and is not recovering from an actual ABI | Greater than about 415 |
| 6 | Has not suffered or recovered from an actual ABI | Less than about 16 |
| | Should be monitored for recovery from an actual ABI | Between a range of about 16 to |
| | | about 425 |
| | Sustained and is not recovering from an actual ABI | Greater than about 425 |
| 7 | Has not suffered or recovered from an actual ABI | Less than about 17 |
| | Should be monitored for recovery from an actual ABI | Between a range of about 17 to |
| | | about 291 |
| | Sustained and is not recovering from an actual ABI | Greater than about 291 |
| 8 | Has not suffered or recovered from an actual ABI | Less than about 17 |
| | Should be monitored for recovery from an actual ABI | Between a range of about 17 to |
| | | about 294 |
| | Sustained and is not recovering from an actual ABI | Greater than about 294 |
| 9 | Has not suffered or recovered from an actual ABI | Less than about 17 |
| | Should be monitored for recovery from an actual ABI | Between a range of about 17 to |
| | | about 307 |
| | Sustained and is not recovering from an actual ABI | Greater than about 307 |
| 10 | Has not suffered or recovered from an actual ABI | Less than about 17 |
| | Should be monitored for recovery from an actual ABI | Between a range of about 17 to |
| | | about 375 |
| | Sustained and is not recovering from an actual ABI | Greater than about 375 |
| 11 | Has not suffered or recovered from an actual ABI | Less than about 17 |
| | Should be monitored for recovery from an actual ABI | Between a range of about 17 to |
| | | about 415 |
| | Sustained and is not recovering from an actual ABI | Greater than about 415 |
| 12 | Has not suffered or recovered from an actual ABI | Less than about 17 |
| | Should be monitored for recovery from an actual ABI | Between a range of about 17 to |
| | | about 425 |
| | Sustained and is not recovering from an actual ABI | Greater than about 425 |
| 13 | Has not suffered or recovered from an actual ABI | Less than about 18 |
| | Should be monitored for recovery from an actual ABI | Between a range of about 18 to |
| | | about 291 |
| | Sustained and is not recovering from an actual ABI | Greater than about 291 |
| 14 | Has not suffered or recovered from an actual ABI | Less than about 18 |
| | Should be monitored for recovery from an actual ABI | Between a range of about 18 to |
| | | about 294 |
| | Sustained and is not recovering from an actual ABI | Greater than about 294 |
| 15 | Has not suffered or recovered from an actual ABI | Less than about 18 |
| | Should be monitored for recovery from an actual ABI | Between a range of about 18 to |
| | | about 307 |
| | Sustained and is not recovering from an actual ABI | Greater than about 307 |
| 16 | Has not suffered or recovered from an actual ABI | Less than about 18 |
| | Should be monitored for recovery from an actual ABI | Between a range of about 18 to |
| | | about 375 |
| | Sustained and is not recovering from an actual ABI | Greater than about 375 |
| 17 | Has not suffered or recovered from an actual ABI | Less than about 18 |
| | Should be monitored for recovery from an actual ABI | Between a range of about 18 to |
| | | about 415 |
| | Sustained and is not recovering from an actual ABI | Greater than about 415 |
| 18 | Has not suffered or recovered from an actual ABI | Less than about 18 |
| | Should be monitored for recovery from an actual ABI | Between a range of about 18 to |
| | | about 425 |
| | Sustained and is not recovering from an actual ABI | Greater than about 425 |

TABLE F
UCH-L1

| | | UCH-L1 level |
|---|---|---|
| Option | Determining that a subject: | (pg/mL) |
| 1 | Has not suffered or recovered from an actual ABI | Less than about 49 |
| | Should be monitored for recovery from an actual ABI | Between a range of about 49 to |
| | | about 243 |
| | Sustained and is not recovering from an actual ABI | Greater than about 243 |
| 2 | Has not suffered or recovered from an actual ABI | Less than about 49 |
| | Should be monitored for recovery from an actual ABI | Between a range of about 49 to |
| | | about 248 |
| | Sustained and is not recovering from an actual ABI | Greater than about 248 |
| 3 | Has not suffered or recovered from an actual ABI | Less than about 49 |
| | Should be monitored for recovery from an actual ABI | Between a range of about 49 to |
| | | about 250 |
| | Sustained and is not recovering from an actual ABI | Greater than about 250 |
| 4 | Has not suffered or recovered from an actual ABI | Less than about 49 |
| | Should be monitored for recovery from an actual ABI | Between a range of about 49 to |
| | | about 267 |
| | Sustained and is not recovering from an actual ABI | Greater than about 267 |
| 5 | Has not suffered or recovered from an actual ABI | Less than about 49 |
| | Should be monitored for recovery from an actual ABI | Between a range of about 49 to |
| | | about 275 |
| | Sustained and is not recovering from an actual ABI | Greater than about 275 |
| 6 | Has not suffered or recovered from an actual ABI | Less than about 51 |
| | Should be monitored for recovery from an actual ABI | Between a range of about 51 to |
| | | about 243 |
| | Sustained and is not recovering from an actual ABI | Greater than about 243 |
| 7 | Has not suffered or recovered from an actual ABI | Less than about 51 |
| | Should be monitored for recovery from an actual ABI | Between a range of about 51 to |
| | | about 248 |
| | Sustained and is not recovering from an actual ABI | Greater than about 248 |
| 8 | Has not suffered or recovered from an actual ABI | Less than about 51 |
| | Should be monitored for recovery from an actual ABI | Between a range of about 49 to |
| | | about 250 |
| | Sustained and is not recovering from an actual ABI | Greater than about 250 |
| 9 | Has not suffered or recovered from an actual ABI | Less than about 51 |
| | Should be monitored for recovery from an actual ABI | Between a range of about 51 to |
| | | about 267 |
| | Sustained and is not recovering from an actual ABI | Greater than about 267 |
| 10 | Has not suffered or recovered from an actual ABI | Less than about 51 |
| | Should be monitored for recovery from an actual ABI | Between a range of about 51 to |
| | | about 275 |
| | Sustained and is not recovering from an actual ABI | Greater than about 275 |
| 11 | Has not suffered or recovered from an actual ABI | Less than about 53 |
| | Should be monitored for recovery from an actual ABI | Between a range of about 53 to |
| | | about 243 |
| | Sustained and is not recovering from an actual ABI | Greater than about 243 |
| 12 | Has not suffered or recovered from an actual ABI | Less than about 53 |
| | Should be monitored for recovery from an actual ABI | Between a range of about 53 to |
| | | about 248 |
| | Sustained and is not recovering from an actual ABI | Greater than about 248 |
| 13 | Has not suffered or recovered from an actual ABI | Less than about 53 |
| | Should be monitored for recovery from an actual ABI | Between a range of about 53 to |
| | | about 250 |
| | Sustained and is not recovering from an actual ABI | Greater than about 250 |
| 14 | Has not suffered or recovered from an actual ABI | Less than about 53 |
| | Should be monitored for recovery from an actual ABI | Between a range of about 53 to |
| | | about 267 |
| | Sustained and is not recovering from an actual ABI | Greater than about 267 |
| 15 | Has not suffered or recovered from an actual ABI | Less than about 53 |
| | Should be monitored for recovery from an actual ABI | Between a range of about 53 to |
| | | about 275 |
| | Sustained and is not recovering from an actual ABI | Greater than about 275 |
| 16 | Has not suffered or recovered from an actual ABI | Less than about 55 |
| | Should be monitored for recovery from an actual ABI | Between a range of about 55 to |
| | | about 243 |
| | Sustained and is not recovering from an actual ABI | Greater than about 243 |
| 17 | Has not suffered or recovered from an actual ABI | Less than about 55 |
| | Should be monitored for recovery from an actual ABI | Between a range of about 55 to |
| | | about 248 |
| | Sustained and is not recovering from an actual ABI | Greater than about 248 |
| 18 | Has not suffered or recovered from an actual ABI | Less than about 55 |
| | Should be monitored for recovery from an actual ABI | Between a range of about 55 to |
| | | about 250 |
| | Sustained and is not recovering from an actual ABI | Greater than about 250 |
| 19 | Has not suffered or recovered from an actual ABI | Less than about 55 |
| | Should be monitored for recovery from an actual ABI | Between a range of about 55 to |
| | | about 267 |
| | Sustained and is not recovering from an actual ABI | Greater than about 267 |
| 20 | Has not suffered or recovered from an actual ABI | Less than about 55 |
| | Should be monitored for recovery from an actual ABI | Between a range of about 55 to |
| | | about 275 |
| | Sustained and is not recovering from an actual ABI | Greater than about 275 |
| 21 | Has not suffered or recovered from an actual ABI | Less than about 56 |
| | Should be monitored for recovery from an actual ABI | Between a range of about 56 to |
| | | about 243 |
| | Sustained and is not recovering from an actual ABI | Greater than about 243 |
| 22 | Has not suffered or recovered from an actual ABI | Less than about 56 |
| | Should be monitored for recovery from an actual ABI | Between a range of about 56 to |
| | | about 248 |
| | Sustained and is not recovering from an actual ABI | Greater than about 248 |
| 23 | Has not suffered or recovered from an actual ABI | Less than about 56 |
| | Should be monitored for recovery from an actual ABI | Between a range of about 56 to |
| | | 250 |
| | Sustained and is not recovering from an actual ABI | Greater than about 250 |
| 24 | Has not suffered or recovered from an actual ABI | Less than about 56 |
| | Should be monitored for recovery from an actual ABI | Between a range of about 56 to |
| | | about 267 |
| | Sustained and is not recovering from an actual ABI | Greater than about 267 |
| 25 | Has not suffered or recovered from an actual ABI | Less than about 56 |
| | Should be monitored for recovery from an actual ABI | Between a range of about 56 to |
| | | about 275 |
| | Sustained and is not recovering from an actual ABI | Greater than about 275 |
| 26 | Has not suffered or recovered from an actual ABI | Less than about 61 |
| | Should be monitored for recovery from an actual ABI | Between a range of about 61 to |
| | | about 243 |
| | Sustained and is not recovering from an actual ABI | Greater than about 243 |
| 27 | Has not suffered or recovered from an actual ABI | Less than about 61 |
| | Should be monitored for recovery from an actual ABI | Between a range of about 61 to |
| | | about 248 |
| | Sustained and is not recovering from an actual ABI | Greater than about 248 |
| 28 | Has not suffered or recovered from an actual ABI | Less than about 61 |
| | Should be monitored for recovery from an actual ABI | Between a range of about 61 to |
| | | about 250 |
| | Sustained and is not recovering from an actual ABI | Greater than about 250 |
| 29 | Has not suffered or recovered from an actual ABI | Less than about 61 |
| | Should be monitored for recovery from an actual ABI | Between a range of about 61 to |
| | | about 267 |
| | Sustained and is not recovering from an actual ABI | Greater than about 267 |
| 30 | Has not suffered or recovered from an actual ABI | Less than about 61 |
| | Should be monitored for recovery from an actual ABI | Between a range of about 61 to |
| | | about 275 |
| | Sustained and is not recovering from an actual ABI | Greater than about 275 |
| 31 | Has not suffered or recovered from an actual ABI | Less than about 63 |
| | Should be monitored for recovery from an actual ABI | Between a range of about 63 to |
| | | about 243 |
| | Sustained and is not recovering from an actual ABI | Greater than about 243 |
| 32 | Has not suffered or recovered from an actual ABI | Less than about 63 |
| | Should be monitored for recovery from an actual ABI | Between a range of about 63 to |
| | | about 248 |
| | Sustained and is not recovering from an actual ABI | Greater than about 248 |
| 33 | Has not suffered or recovered from an actual ABI | Less than about 63 |
| | Should be monitored for recovery from an actual ABI | Between a range of about 63 to |
| | | about 250 |
| | Sustained and is not recovering from an actual ABI | Greater than about 250 |
| 34 | Has not suffered or recovered from an actual ABI | Less than about 63 |
| | Should be monitored for recovery from an actual ABI | Between a range of about 63 to |
| | | about 267 |
| | Sustained and is not recovering from an actual ABI | Greater than about 267 |
| 35 | Has not suffered or recovered from an actual ABI | Less than about 63 |
| | Should be monitored for recovery from an actual ABI | Between a range of about 63 to |
| | | about 275 |
| | Sustained and is not recovering from an actual ABI | Greater than about 275 |

TABLE G
GFAP

| Option | Determining that a subject: | GFAP level (pg/mL) | Measure |
|---|---|---|---|
| 1 | Has not suffered or recovered from | Less than about 17 | GCS |
| | an actual ABI | | (such as a mild TBI; GCS 13- |
| | | | 15) |
| | Should be monitored for recovery | Between a range of | GCS |
| | from an actual ABI | about 17 to about | (such as a moderate, severe, |
| | | 425 | or moderate-to-severe TBI; |
| | | | GCS 3-12) |
| | Sustained and is not recovering | Greater than about | GCS |
| | from an actual ABI | 425 | (such as a moderate, severe, |
| | | | or moderate-to-severe TBI; |
| | | | GCS 3-12) |
| 2 | Has not suffered or recovered from | Less than about 17 | GCS |
| | an actual ABI | | (such as a mild TBI; GCS 13- |
| | | | 15) |
| | Should be monitored for recovery | Between a range of | GCS |
| | from an actual ABI | about 17 to about | (such as a moderate, severe, |
| | | 431 | or moderate-to-severe TBI; |
| | | | GCS 3-12) |
| | Sustained and is not recovering | Greater than about | GCS |
| | from an actual ABI | 431 | (such as a moderate, severe, |
| | | | or moderate-to-severe TBI; |
| | | | GCS 3-12) |
| 3 | Has not suffered or recovered from | Less than about 17 | GCS |
| | an actual ABI | | (such as a mild TBI; GCS 13- |
| | | | 15) |
| | Should be monitored for recovery | Between a range of | GCS |
| | from an actual ABI | about 17 to about | (such as a moderate, severe, |
| | | 415 | or moderate-to-severe TBI; |
| | | | GCS 3-12) |
| | Sustained and is not recovering | Greater than about | GCS |
| | from an actual ABI | 415 | (such as a moderate, severe, |
| | | | or moderate-to-severe TBI; |
| | | | GCS 3-12) |
| 4 | Has not suffered or recovered from | Less than about 17 | GOSE |
| | an actual ABI | | (such as a GOSE score of 4- |
| | | | 8) |
| | Should be monitored for recovery | Between a range of | GOSE |
| | from an actual ABI | about 17 to about | (such as a GOSE score of 0- |
| | | 431 | 3) |
| | Sustained and is not recovering | Greater than about | GOSE |
| | from an actual ABI | 431 | (such as a GOSE score of 0- |
| | | | 3) |
| 5 | Has not suffered or recovered from | Less than about 17 | GOSE |
| | an actual ABI | | (such as a GOSE score of 4- |
| | | | 8) |
| | Should be monitored for recovery | Between a range of | GOSE |
| | from an actual ABI | about 17 to about | (such as a GOSE score of 0- |
| | | 375 | 3) |
| | Sustained and is not recovering | Greater than about | GOSE |
| | from an actual ABI | 375 | (such as a GOSE score of 0- |
| | | | 3) |
| 6 | Has not suffered or recovered from | Less than about 17 | CT |
| | an actual ABI | | (such as a negative head CT) |
| | Should be monitored for recovery | Between a range of | CT |
| | from an actual ABI | about 17 to about | (such as a positive head CT, |
| | | 307 | a negative head CT, or no |
| | | | head CT) |
| | Sustained and is not recovering | Greater than about | CT |
| | from an actual ABI | 307 | (such as a positive head CT, |
| | | | a negative head CT, or no |
| | | | head CT) |
| 7 | Has not suffered or recovered from | Less than about 18 | CT |
| | an actual ABI | | (such as a negative head CT) |
| | Should be monitored for recovery | Between a range of | CT |
| | from an actual ABI | about 18 to about | (such as a positive head CT, |
| | | 294 | a negative head CT, or no |
| | | | head CT) |
| | Sustained and is not recovering | Greater than about | CT |
| | from an actual ABI | 294 | (such as a positive head CT, |
| | | | a negative head CT, or no |
| | | | head CT) |
| 8 | Has not suffered or recovered from | Less than about 16 | CT |
| | an actual ABI | | (such as a negative head CT) |
| | Should be monitored for recovery | Between a range of | CT |
| | from an actual ABI | about 16 to about | (such as a positive head CT, |
| | | 291 | a negative head CT, or no |
| | | | head CT) |
| | Sustained and is not recovering | Greater than about | CT |
| | from an actual ABI | 291 | (such as a positive head CT, |
| | | | a negative head CT, or no |
| | | | head CT) |

TABLE H
UCH-L1

| Option | Determining that a subject: | UCH-L1 level (pg/mL) | Measure |
|---|---|---|---|
| 1 | Has not suffered or recovered from | Less than about 56 | GCS |
| | an actual ABI | | (such as a mild TBI; GCS 13- |
| | | | 15) |
| | Should be monitored for recovery | Between a range of | GCS |
| | from an actual ABI | about 56 to about | (such as a moderate, severe, |
| | | 267 | or moderate-to-severe TBI; |
| | | | GCS 3-12) |
| | Sustained and is not recovering | Greater than about | GCS |
| | from an actual ABI | 267 | (such as a moderate, severe, |
| | | | or moderate-to-severe TBI; |
| | | | GCS 3-12) |
| 2 | Has not suffered or recovered from | Less than about 61 | GCS |
| | an actual ABI | | (such as a mild TBI; GCS 13- |
| | | | 15) |
| | Should be monitored for recovery | Between a range of | GCS |
| | from an actual ABI | about 61 to about | (such as a moderate, severe, |
| | | 248 | or moderate-to-severe TBI; |
| | | | GCS 3-12) |
| | Sustained and is not recovering | Greater than about | GCS |
| | from an actual ABI | 248 | (such as a moderate, severe, |
| | | | or moderate-to-severe TBI; |
| | | | GCS 3-12) |
| 3 | Has not suffered or recovered from | Less than about 56 | GCS |
| | an actual ABI | | (such as a mild TBI; GCS 13- |
| | | | 15) |
| | Should be monitored for recovery | Between a range of | GCS |
| | from an actual ABI | about 56 to about | (such as a moderate, severe, |
| | | 250 | or moderate-to-severe TBI; |
| | | | GCS 3-12) |
| | Sustained and is not recovering | Greater than about | GCS |
| | from an actual ABI | 250 | (such as a moderate, severe, |
| | | | or moderate-to-severe TBI; |
| | | | GCS 3-12) |
| 4 | Has not suffered or recovered from | Less than about 49 | GOSE |
| | an actual ABI | | (such as a GOSE score of 4-8) |
| | Should be monitored for recovery | Between a range of | GOSE |
| | from an actual ABI | about 49 to about | (such as a GOSE score of 0-3) |
| | | 275 | |
| | Sustained and is not recovering | Greater than about | GOSE |
| | from an actual ABI | 275 | (such as a GOSE score of 0-3) |
| 5 | Has not suffered or recovered from | Less than about 63 | GOSE |
| | an actual ABI | | (such as a GOSE score of 4-8) |
| | Should be monitored for recovery | Between a range of | GOSE |
| | from an actual ABI | about 63 to about | (such as a GOSE score of 0-3) |
| | | 267 | |
| | Sustained and is not recovering | Greater than about | GOSE |
| | from an actual ABI | 267 | (such as a GOSE score of 0-3) |
| 6 | Has not suffered or recovered from | Less than about 53 | GOSE |
| | an actual ABI | | (such as a GOSE score of 4-8) |
| | Should be monitored for recovery | Between a range of | GOSE |
| | from an actual ABI | about 53 to about | (such as a GOSE score of 0-3) |
| | | 275 | |
| | Sustained and is not recovering | Greater than about | GOSE |
| | from an actual ABI | 275 | (such as a GOSE score of 0-3) |
| 7 | Has not suffered or recovered from | Less than about 55 | CT |
| | an actual ABI | | (such as a negative head CT) |
| | Should be monitored for recovery | Between a range of | CT |
| | from an actual ABI | about 55 to about | (such as a positive head CT, a |
| | | 250 | negative head CT, or no head |
| | | | CT) |
| | Sustained and is not recovering | Greater than about | CT |
| | from an actual ABI | 250 | (such as a positive head CT, a |
| | | | negative head CT, or no head |
| | | | CT) |
| 8 | Has not suffered or recovered from | Less than about 51 | CT |
| | an actual ABI | | (such as a negative head CT) |
| | Should be monitored for recovery | Between a range of | CT |
| | from an actual ABI | about 51 to about | (such as a positive head CT, a |
| | | 248 | negative head CT, or no head |
| | | | CT) |
| | Sustained and is not recovering | Greater than about | CT |
| | from an actual ABI | 248 | (such as a positive head CT, a |
| | | | negative head CT, or no head |
| | | | CT) |
| 9 | Has not suffered or recovered from | Less than about 55 | CT |
| | an actual ABI | | (such as a negative head CT) |
| | Should be monitored for recovery | Between a range of | CT |
| | from an actual ABI | about 55 to about | (such as a positive head CT, a |
| | | 243 | negative head CT, or no head |
| | | | CT) |
| | Sustained and is not recovering | Greater than about | CT |
| | from an actual ABI | 243 | (such as a positive head CT, a |
| | | | negative head CT, or no head |
| | | | CT) |

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the present disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the present disclosure, may be made without departing from the spirit and scope thereof.

For reasons of completeness, various aspects of the present disclosure are set out in the following numbered clauses:

Clause 1. A method for aiding in the diagnosis and evaluation of a subject that has sustained or may have sustained an acquired brain injury (ABI), the method comprising:
a. performing one or more assays on at least one or more samples obtained from the subject within after about twenty-four hours to (i) about one week, (ii) about two weeks, or (iii) about three weeks, after an actual or suspected ABI to measure or detect a level of a biomarker in the sample, said biomarker comprising glial fibrillary acidic protein (GFAP), ubiquitin carboxy-terminal hydrolase L1 (UCH-L1), or a combination of GFAP and UCH-L1; and
b. determining that the subject:
i) has not suffered or recovered from an actual ABI if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

Clause 2. The method of clause 1, wherein:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

Clause 3. The method of clause 1 or clause 2, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (c) any combinations of (a)-(d).

Clause 3A. The method of clause 1, wherein:
in b i) (a) the GFAP level is less than about 18 pg/mL; (b) the UCH-L1 level is less than about 63 pg/mL; or (c) the GFAP level is less than about 18 pg/mL and the UCH-L1 level is less than about 63 pg/mL;
in b ii), (a) the GFAP level is between about 18 pg/mL at the lower limit up to about 431 pg/mL at the upper limit; (b) the UCH-L1 level is between about 63 pg/mL at the lower limit and about 275 pg/mL at the upper limit; or (c) the GFAP level is between about 18 pg/mL at the lower limit up to about 431 pg/mL at the upper limit and the UCH-L1 level is between about 63 pg/mL at the lower limit and about 275 pg/mL at the upper limit; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 3B. The method of clause 3A, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 4. The method of clause 1, wherein:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Clause 4A. The method of clause 1, wherein:
in b i) (a) the GFAP level is less than about 18 pg/mL; (b) UCH-L1 level is less than about 55 pg/mL; or (c) GFAP level is less than about 18 pg/mL and UCH-L1 level is less than about 55 pg/mL;
in b ii), (a) the GFAP level is between a range of about 18 pg/mL to about 307 pg/mL; (b) the UCH-L1 level is between a range of about 55 pg/mL to about 250 pg/mL; or (c) the GFAP level is between a range of about 18 pg/mL to about 307 pg/mL and the UCH-L1 level is between a range of about 55 pg/mL to about 250 pg/mL; or
in b iii), (a) the GFAP level is greater than about 307 pg/mL; (b) the UCH-L1 level is greater than about 250 pg/mL; (c) the GFAP level is greater than about 307 pg/mL and the UCH-L1 level is greater than about 250 pg/mL.

Clause 5. The method of clause 4, wherein (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

Clause 5A. The method of clause 4A, wherein (1) each of b i) (a), b i) (b) and b) i) (c) are correlated with a level associated with a negative head CT; (2) each of b ii) (a), b ii) (b), and b ii) (c) are correlated with a positive head CT; or (3) each of b iii) (a), b iii) (b), and b iii) (c) are correlated with a positive head CT.

Clause 6. The method of clause 1, wherein:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL.

Clause 6A. The method of clause 1, wherein:
in b i) (a) the GFAP level is less than about 17 pg/mL; (b) UCH-L1 level is less than about 61 pg/mL; or (c) GFAP level is less than about 17 pg/mL and UCH-L1 level is less than about 61 pg/mL;
in b ii), (a) the GFAP level is between a range of about 17 pg/mL to about 431 pg/mL; (b) the UCH-L1 level is between a range of about 61 pg/mL to about 267 pg/mL; or (c) the GFAP level is between a range of about 17 pg/mL to about 431 pg/mL and the UCH-L1 level is between a range of about 61 pg/mL to about 267 pg/mL; or
in b iii), (a) the GFAP level is greater than about 431 pg/mL; (b) the UCH-L1 level is greater than about 267 pg/mL; (c) the GFAP level is greater than about 431 pg/mL and the UCH-L1 level is greater than about 267 pg/mL.

Clause 7. The method of clause 6, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 7A. The method of clause 6A, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 8. The method of clause 1, wherein:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Clause 8A. The method of clause 1, wherein:
in b i) (a) the GFAP level is less than about 17 pg/mL; (b) UCH-L1 level is less than about 63 pg/mL; or (c) GFAP level is less than about 17 pg/mL and UCH-L1 level is less than about 63 pg/mL;
in b ii), (a) the GFAP level is between a range of about 17 pg/mL to about 431 pg/mL; (b) the UCH-L1 level is between a range of about 63 pg/mL to about 275 pg/mL; or (c) the GFAP level is between a range of about 17 pg/mL to about 431 pg/mL and the UCH-L1 is between a range of about 63 pg/mL to about 275 pg/mL; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 9. The method of clause 8, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3

Clause 9A. The method of clause 8A, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 10. The method of any of clauses 1-9, wherein measuring the GFAP level, UCH-L1 level, or GFAP level and UCH-L1 level is done by immunoassay or a clinical chemistry assay.

Clause 11. The method of any of clauses 1-10, wherein the assay is performed using a point-of-care assay or single molecule detection.

Clause 12. The method of any of clauses 1-11, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 13. The method of any of clauses 1-12, wherein the actual or suspected ABI is caused by physical shaking, blunt impact by an external mechanical or other force that results in a closed or open head trauma, one or more falls, explosions or blasts or other types of blunt force trauma.

Clause 14. The method of any of clauses 1-13, wherein the actual or suspected ABI is a traumatic brain injury.

Clause 15. The method of any of clauses 1-14, wherein the actual or suspected ABI is caused by fire exposure, ingestion of a chemical or toxin, or a combination of fire exposure, and ingestion of a chemical or toxin.

Clause 16. The method of clause 15, wherein the chemical or toxin is mold, asbestos, a pesticide, an insecticide, an organic solvent, a paint, a glue, a gas, an organic metal, a drug of abuse or one or more combinations thereof.

Clause 17. The method of any of clauses 1-14, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 18. The method of any of clauses 1-17, wherein the blood sample is whole blood, serum or plasma, either venous blood or capillary blood.

Clause 18A. The method of any of clause 18, wherein the blood sample is obtained from venous blood and is whole blood, serum, or plasma.

Clause 18B. The method of claim 18A, wherein the blood sample is a whole blood sample.

Clause 18C. The method of claim 18A, wherein the blood sample is serum.

Clause 18D. The method of claim 18A, wherein the blood sample is plasma.

Clause 18E. The method of clause 18, wherein the blood sample is obtained from capillary blood and is whole blood, serum, or plasma.

Clause 18F. The method of clause 18E, wherein the blood sample is a whole blood sample.

Clause 18G. The method of clause 18E, wherein the blood sample is serum.

Clause 18H. The method of clause 18E, wherein the blood sample is plasma.

Clause 19. The method of any of clauses 1-18, wherein the subject is a human subject.

Clause 20. A method for aiding in the diagnosis and evaluation of a subject that has sustained or may have sustained an acquired brain injury (ABI), the method comprising:
a. obtaining a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI; and
b. determining that the subject:
i) has not suffered or has recovered from an actual ABI if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

Clause 21. The method of clause 20, wherein:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

Clause 22. The method of clause 20 or clause 21, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 22A. The method of clause 20, wherein:
in b i) (a) the GFAP level is less than about 18 pg/mL; (b) the UCH-L1 level is less than about 63 pg/mL; or (c) the GFAP level is less than about 18 pg/mL and the UCH-L1 level is less than about 63 pg/mL;
in b ii), (a) the GFAP level is between about 18 pg/mL at the lower limit up to about 431 pg/mL at the upper limit; (b) the UCH-L1 level is between about 63 pg/mL at the lower limit and about 275 pg/mL at the upper limit; or (c) the GFAP level is between about 18 pg/mL at the lower limit up to about 431 pg/mL at the upper limit and the UCH-L1 level is between about 63 pg/mL at the lower limit and about 275 pg/mL at the upper limit; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 22B. The method of clause 22A, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 23. The method of clause 20, wherein:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Clause 23A. The method of clause 20, wherein:
in b i) (a) the GFAP level is less than about 18 pg/mL; (b) UCH-L1 level is less than about 55 pg/mL; or (c) GFAP level is less than about 18 pg/mL and UCH-L1 level is less than about 55 pg/mL;
in b ii), (a) the GFAP level is between a range of about 18 pg/mL to about 307 pg/mL; (b) the UCH-L1 level is between a range of about 55 pg/mL to about 250 pg/mL; or (c) the GFAP level is between a range of about 18 pg/mL to about 307 pg/mL and the UCH-L1 level is between a range of about 55 pg/mL to about 250 pg/mL; or
in b iii), (a) the GFAP level is greater than about 307 pg/mL; (b) the UCH-L1 level is greater than about 250 pg/mL; (c) the GFAP level is greater than about 307 pg/mL and the UCH-L1 level is greater than about 250 pg/mL.

Clause 24. The method of clause 23, wherein (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

Clause 24A. The method of clause 23A, wherein (1) each of b i) (a), b i) (b) and b) i) (c) are correlated with a level associated with a negative head CT; (2) each of b ii) (a), b ii) (b), and b ii) (c) are correlated with a positive head CT; or (3) each of b iii) (a), b iii) (b), and b iii) (c) are correlated with a positive head CT.

Clause 25. The method of clause 20, wherein:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL.

Clause 25A. The method of clause 20, wherein:
in b i) (a) the GFAP level is less than about 17 pg/mL; (b) UCH-L1 level is less than about 61 pg/mL; or (c) GFAP level is less than about 17 pg/mL and UCH-L1 level is less than about 61 pg/mL;
in b ii), (a) the GFAP level is between a range of about 17 pg/mL to about 431 pg/mL; (b) the UCH-L1 level is between a range of about 61 pg/mL to about 267 pg/mL; or (c) the GFAP level is between a range of about 17 pg/mL to about 431 pg/mL and the UCH-L1 level is between a range of about 61 pg/mL to about 267 pg/mL; or
in b iii), (a) the GFAP level is greater than about 431 pg/mL; (b) the UCH-L1 level is greater than about 267 pg/mL; (c) the GFAP level is greater than about 431 pg/mL and the UCH-L1 level is greater than about 267 pg/mL.

Clause 26. The method of clause 25, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 26A. The method of clause 25A, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 27. The method of clause 20, wherein:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Clause 27A. The method of clause 20, wherein:
in b i) (a) the GFAP level is less than about 17 pg/mL; (b) UCH-L1 level is less than about 63 pg/mL; or (c) GFAP level is less than about 17 pg/mL and UCH-L1 level is less than about 63 pg/mL;
in b ii), (a) the GFAP level is between a range of about 17 pg/mL to about 431 pg/mL; (b) the UCH-L1 level is between a range of about 63 pg/mL to about 275 pg/mL; or (c) the GFAP level is between a range of about 17 pg/mL to about 431 pg/mL and the UCH-L1 is between a range of about 63 pg/mL to about 275 pg/mL; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 28. The method of clause 27, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 28A. The method of clause 27A, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 29. The method of any of clauses 20-28, wherein measuring the GFAP level, UCH-L1 level, or GFAP level and UCH-L1 level is done by immunoassay or a clinical chemistry assay.

Clause 30. The method of any of clauses 20-29, wherein the assay is a point-of-care assay or single molecule detection.

Clause 31. The method of any of clauses 20-30, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 32. The method of any of clauses 20-31, wherein the actual or suspected ABI is caused by physical shaking, blunt impact by an external mechanical or other force that results in a closed or open head trauma, one or more falls, explosions or blasts or other types of blunt force trauma.

Clause 33. The method of any of clauses 20-32, wherein the actual or suspected ABI is a traumatic brain injury.

Clause 34. The method of any of clauses 20-33, wherein the actual or suspected ABI is caused by fire exposure, ingestion of a chemical or toxin, or a combination of fire exposure, and ingestion of a chemical or toxin.

Clause 35. The method of clause 34, wherein the chemical or toxin is mold, asbestos, a pesticide, an insecticide, an organic solvent, a paint, a glue, a gas, an organic metal, a drug of abuse or one or more combinations thereof.

Clause 36. The method of any of clauses 20-33, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 37. The method of any of clauses 20-36, wherein the blood sample is whole blood, serum or plasma, either venous blood or capillary blood.

Clause 38. The method of any of clause 37, wherein the blood sample is obtained from venous blood and is whole blood, serum, or plasma.

Clause 38A. The method of claim 38, wherein the blood sample is a whole blood sample.

Clause 38B. The method of claim 38, wherein the blood sample is serum.

Clause 38C. The method of claim 38, wherein the blood sample is plasma.

Clause 39. The method of clause 37, wherein the blood sample is obtained from capillary blood and is whole blood, serum, or plasma.

Clause 39A. The method of clause 39, wherein the blood sample is a whole blood sample.

Clause 39B. The method of clause 39, wherein the blood sample is serum.

Clause 39C. The method of clause 39, wherein the blood sample is plasma.

Clause 40. The method of any of clauses 20-39, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 40A. The method of clause 40, wherein the blood sample is obtained at one or multiple timepoints after about twenty-five hours.

Clause 40B. The method of clause 40, wherein the blood sample is obtained at one or more multiple timepoints after about thirty hours.

Clause 40C. The method of clause 40, wherein the blood sample is obtained at one or more multiple timepoints after about thirty-five hours.

Clause 40D. The method of clause 40, wherein the blood sample is obtained at one or more multiple timepoints after about forty hours.

Clause 40E. The method of clause 40, wherein the blood sample is obtained at one or more multiple timepoints after about two days.

Clause 40F. The method of clause 40, wherein the blood sample is obtained at one or more multiple timepoints after about five days.

Clause 40G. The method of clause 40, wherein the blood sample is obtained at one or more multiple timepoints after about one week.

Clause 40H. The method of clause 40, wherein the blood sample is obtained at one or more multiple timepoints after about one week.

Clause 40I. The method of clause 40, wherein the blood sample is obtained at one or more multiple timepoints after about two weeks.

Clause 40J. The method of clause 40, wherein the blood sample is obtained at one or more multiple timepoints after about three weeks.

Clause 41. The method of any of clauses 20-40, wherein the subject is a human subject.

Clause 42. A method for aiding in the diagnosis and evaluation of a subject that has sustained or may have sustained an acquired brain injury (ABI), the method comprising:
a. performing one or more assays on at least one or more samples obtained from the subject within after about twenty-four hours to (i) about one week, (ii) about two weeks, or (iii) about three weeks, after an actual or suspected ABI to measure or detect a level of a biomarker in the sample, said biomarker comprising glial fibrillary acidic protein (GFAP);
b. determining that the subject:
i) has not suffered or recovered from an actual ABI if the subject's GFAP level is less than from about 16 pg/mL to about 18 pg/mL;
ii) should be monitored for recovery from an actual ABI if the subject's GFAP level is between a range of from about 16 pg/mL to about 18 pg/mL at the lower limit up to from about 291 pg/mL to about 431 pg/mL at the upper limit; or
iii) sustained and is not recovering from an actual ABI if the subject's GFAP level is greater than from about 291 pg/mL to about 431 pg/mL.

Clause 43. The method of clause 42, wherein:
in b i), the subject's GFAP level is less than about 16 pg/mL;
in b ii), the subject's GFAP level is between a range of about 16 pg/mL to about 431 pg/mL; or in b iii), the subject's GFAP level is greater than about 431 pg/mL.

Clause 44. The method of clause 42 or clause 43, wherein the GFAP level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 44A. The method of clause 42, wherein:
in b i), the GFAP level is less than about 18 pg/mL;
in b ii), (a) the GFAP level is between about 18 pg/mL at the lower limit up to about 431 pg/mL at the upper limit; or in b iii), (a) the GFAP level is greater than about 431 pg/mL.

Clause 44B. The method of clause 44A, wherein the GFAP level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 45. The method of clause 42, wherein:
in b i), the GFAP level is less than from about 16 pg/mL to about 18 pg/mL;
in b ii), the GFAP level is between a range of about 16 pg/mL to about 291 pg/mL, about 17 pg/mL to about 307 pg/mL, about 18 pg/mL to about 294 pg/mL; or
in b iii), the GFAP level is greater than about 291 pg/mL, about 294 pg/mL, or about 307 pg/mL.

Clause 45A. The method of clause 42, wherein:
in b i), the GFAP level is less than about 18 pg/mL;
in b ii), the GFAP level is between a range of about 18 pg/mL to about 307 pg/mL; or in b iii), the GFAP level is greater than about 307 pg/mL.

Clause 46. The method of clause 45, wherein (1) b i) is correlated with a level associated with a negative head CT; (2) b ii) is correlated with a positive head CT, a negative head CT, or no head CT; or (3) b iii) is correlated with a positive head CT, a negative head CT or no head CT.

Clause 46A. The method of clause 45A, wherein 1) b i) is correlated with a level associated with a negative head CT; (2) b ii) is correlated with a positive head CT; or (3) b iii) is correlated with a positive head CT.

Clause 47. The method of clause 42, wherein:
in b i), the GFAP level is less than from about 17 pg/mL;
in b ii), the GFAP level is between a range of about 17 pg/mL to about 415 pg/mL, about 17 pg/mL to about 425 pg/mL, or about 17 pg/mL to about 431 pg/mL; or
in b iii), the GFAP level is greater than about 415 pg/mL, about 425 pg/mL, or about 431 pg/mL.

Clause 47A. The method of clause 42, wherein:
in b i), the GFAP level is less than about 17 pg/mL;
in b ii), the GFAP level is between a range of about 17 pg/mL to about 431 pg/mL; or in b iii), (a) the GFAP level is greater than about 431 pg/mL.

Clause 48. The method of clause 47, wherein (1) b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii) is correlated with a GCS score severity for a moderate, severe, or a moderate-to-severe TBI (GCS 3-12).

Clause 48A. The method of clause 47A, wherein (1) b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii) is correlated with a GCS score severity for a moderate, severe, or a moderate-to-severe TBI (GCS 3-12).

Clause 49. The method of clause 42, wherein:
in b i), the GFAP level is less than from about 17 pg/mL;
in b ii), the GFAP level is between a range of about 17 pg/mL to about 375 pg/mL or about 17 pg/mL to about 431 pg/mL; or
in b iii), the GFAP level is greater than about 375 pg/mL or about 431 pg/mL.

Clause 49A. The method of clause 42, wherein:
in b i), the GFAP level is less than about 17 pg/mL;
in b ii), the GFAP level is between a range of about 17 pg/mL to about 431 pg/mL; or
in b iii), (a) the GFAP level is greater than about 431 pg/mL.

Clause 50. The method of clause 49, wherein (1) b i) is correlated with a level associated with a GOSE score of 4-8; (2) b ii) is correlated with a level associated with a GOSE score of 0-3; or (3) b iii) is correlated with a level associated with a GOSE score of 0-3.

Clause 50A. The method of clause 49A, wherein (1) b i) is correlated with a level associated with a GOSE score of 4-8; (2) b ii) is correlated with a level associated with a GOSE score of 0-3; or (3) each of b iii) (a), b iii) (b), and b iii) is correlated with a level associated with a GOSE score of 0-3.

Clause 51. The method of any of clauses 42-50, wherein measuring the GFAP level is done by immunoassay or a clinical chemistry assay.

Clause 52. The method of any of clauses 42-51, wherein the assay is a point-of-care assay or single molecule detection.

Clause 53. The method of any of clauses 42-52, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 54. The method of any of clauses 42-53, wherein the actual or suspected ABI is caused by physical shaking, blunt impact by an external mechanical or other force that results in a closed or open head trauma, one or more falls, explosions or blasts or other types of blunt force trauma.

Clause 55. The method of any of clauses 42-54, wherein the actual or suspected ABI is a traumatic brain injury.

Clause 56. The method of any of clauses 42-55, wherein the actual or suspected ABI is caused by fire exposure, ingestion of a chemical or toxin, or a combination of fire exposure, and ingestion of a chemical or toxin.

Clause 57. The method of clause 56, wherein the chemical or toxin is mold, asbestos, a pesticide, an insecticide, an organic solvent, a paint, a glue, a gas, an organic metal, a drug of abuse or one or more combinations thereof.

Clause 58. The method of any of clauses 42-55, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 59. The method of any of clauses 42-58, wherein the blood sample is whole blood, serum or plasma, either venous blood or capillary blood.

Clause 60. The method of clause 59, wherein the blood sample is obtained from venous blood and is whole blood, serum, or plasma.

Clause 60A. The method of claim 60, wherein the blood sample is a whole blood sample.

Clause 60B. The method of claim 60, wherein the blood sample is serum.

Clause 60C. The method of claim 60, wherein the blood sample is plasma.

Clause 61. The method of clause 59, wherein the blood sample is obtained from capillary blood and is whole blood, serum, or plasma.

Clause 61A. The method of clause 61, wherein the blood sample is a whole blood sample.

Clause 61B. The method of clause 61, wherein the blood sample is serum.

Clause 61C. The method of clause 61, wherein the blood sample is plasma.

Clause 62. The method of any of clauses 42-61, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 62A. The method of clause 62, wherein the blood sample is obtained at one or multiple timepoints after about twenty-five hours.

Clause 62B. The method of clause 62, wherein the blood sample is obtained at one or more multiple timepoints after about thirty hours.

Clause 62C. The method of clause 62, wherein the blood sample is obtained at one or more multiple timepoints after about thirty-five hours.

Clause 62D. The method of clause 62, wherein the blood sample is obtained at one or more multiple timepoints after about forty hours.

Clause 62E. The method of clause 62, wherein the blood sample is obtained at one or more multiple timepoints after about two days.

Clause 62F. The method of clause 62, wherein the blood sample is obtained at one or more multiple timepoints after about five days.

Clause 62G. The method of clause 62, wherein the blood sample is obtained at one or more multiple timepoints after about one week.

Clause 62H. The method of clause 62, wherein the blood sample is obtained at one or more multiple timepoints after about one week.

Clause 62I. The method of clause 62, wherein the blood sample is obtained at one or more multiple timepoints after about two weeks.

Clause 62J. The method of clause 62, wherein the blood sample is obtained at one or more multiple timepoints after about three weeks.

Clause 63. The method of any of clauses 42-62, wherein the subject is a human subject.

Clause 64. A method for aiding in the diagnosis and evaluation of a subject that has sustained or may have sustained an acquired brain injury (ABI), the method comprising:
a. performing one or more assays on at least one or more samples obtained from the subject within after about twenty-four hours to (i) about one week, (ii) about two weeks, or (iii) about three weeks, after an actual or suspected ABI to measure or detect a level of a biomarker in the sample, said biomarker comprising ubiquitin carboxy-terminal hydrolase L1 (UCH-L1);
b. determining that the subject:
i) has not suffered or recovered from an actual ABI if the subject's UCH-L1 level is less than from about 49 pg/mL to about 63 pg/mL;
ii) should be monitored for recovery from an actual ABI if the subject's UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's UCH-L1 level is greater than from about 243 pg/mL to about 275 pg/mL.

Clause 65. The method of clause 64, wherein:
in b i): the subject's UCH-L1 level is less than about 49 pg/mL;
in b ii), the subject's UCH-L1 level is between a range of about 49 pg/mL to about 275 pg/mL; or
in b iii) the subject's UCH-L1 level is greater than about 275 pg/mL.

Clause 66. The method of clause 64 or clause 65, wherein the UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 66A. The method of clause 64, wherein:
in b i), the UCH-L1 level is less than about 63 pg/mL;
in b ii), the UCH-L1 level is between about 63 pg/mL at the lower limit and about 275 pg/mL at the upper limit; or
in b iii), the UCH-L1 level is greater than about 275 pg/mL.

Clause 66B. The method of clause 66A, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 67. The method of clause 66, wherein:
in bi), UCH-L1 level is less than about 51 pg/mL or about 55 pg/mL;
in b ii), the UCH-L1 level is between about 51 pg/mL to about 248 pg/mL, about 55 pg/mL to about 243 pg/mL, or about 55 pg/mL to about 250 pg/mL; or
in b iii), the UCH-L1 level is greater than about 243 pg/mL, about 248 pg/mL, or about 250 pg/mL.

Clause 67A. The method of clause 64, wherein:
in b i), the UCH-L1 level is less than about 55 pg/mL;
in b ii), the UCH-L1 level is between a range of about 55 pg/mL to about 250 pg/mL; or
in b iii), the UCH-L1 level is greater than about 250 pg/mL.

Clause 68. The method of clause 67, wherein (1) b i) is correlated with a level associated with a negative head CT; (2) b ii) is correlated with a positive head CT, a negative head CT, or no head CT; or (3) b iii) is correlated with a positive head CT, a negative head CT or no head CT.

Clause 68A. The method of clause 67A, wherein (1) b i) is correlated with a level associated with a negative head CT; (2) b ii) is correlated with a positive head CT; or (3) b iii) is correlated with a positive head CT.

Clause 69. The method of clause 64, wherein:
in b i), the UCH-L1 level is less than about 51 pg/mL or about 55 pg/mL;
in b ii), the UCH-L1 level is between about 56 pg/mL to about 250 pg/mL, about 56 pg/mL to about 267 pg/mL, or about 61 pg/mL to about 248 pg/mL; or
in b iii), the UCH-L1 level is greater than about 248 pg/mL, about 250 pg/mL, or about 267 pg/mL.

Clause 69A. The method of clause 64, wherein:
in b i), the UCH-L1 level is less than about 61 pg/mL;
in b ii), the UCH-L1 level is between a range of about 61 pg/mL to about 267 pg/mL; or
in b iii), the UCH-L1 level is greater than about 267 pg/mL.

Clause 70. The method of clause 69, wherein (1) b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii is correlated with a GCS score severity for a moderate, severe, or a moderate-to-severe TBI (GCS 3-12).

Clause 70A. The method of clause 69A, wherein (1) each of b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii) is correlated with a GCS score severity for a moderate, severe, or a moderate-to-severe TBI (GCS 3-12).

Clause 71. The method of clause 64, wherein:
in b i), the UCH-L1 level is less than about 49 pg/mL, about 53 pg/mL, or about 63 pg/mL;
in b ii), the UCH-L1 level is between about 49 pg/mL to about 275 pg/mL, about 53 pg/mL to about 275 pg/mL, or about 63 pg/mL to about 267 pg/mL; or
in b iii), the UCH-L1 level is greater than about 275 pg/mL or about 267 pg/mL.

Clause 71A. The method of claim 64, wherein:
in b i), the UCH-L1 level is less than about 63 pg/ml;
in b ii), the UCH-L1 level is between a range of about 63 pg/mL to about 275 pg/mL; or
in b iii), the UCH-L1 level is greater than about 275 pg/mL.

Clause 72. The method of clause 71, wherein (1) b i) is correlated with a level associated with a GOSE score of 4-8; (2) b ii) is correlated with a level associated with a GOSE score of 0-3; or (3) b iii) is correlated with a level associated with a GOSE score of 0-3.

Clause 72A. The method of clause 71A, wherein (1) b i) is correlated with a level associated with a GOSE score of 4-8; (2) b ii) is correlated with a level associated with a GOSE score of 0-3; or (3) b iii) (a), b iii) is correlated with a level associated with a GOSE score of 0-3.

Clause 73. The method of any of clauses 64-72, wherein measuring the UCH-L1 level is done by immunoassay or a clinical chemistry assay.

Clause 74. The method of any of clauses 64-73, wherein the assay is a point-of-care assay or single molecule detection.

Clause 75. The method of any of clauses 64-74, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 76. The method of any of clauses 64-75, wherein the actual or suspected ABI is caused by physical shaking, blunt impact by an external mechanical or other force that results in a closed or open head trauma, one or more falls, explosions or blasts or other types of blunt force trauma.

Clause 77. The method of any of clauses 64-76, wherein the actual or suspected ABI is a traumatic brain injury.

Clause 78. The method of any of clauses 64-77, wherein the actual or suspected ABI is caused by fire exposure, ingestion of a chemical or toxin, or a combination of fire exposure, and ingestion of a chemical or toxin.

Clause 79. The method of clause 78, wherein the chemical or toxin is mold, asbestos, a pesticide, an insecticide, an organic solvent, a paint, a glue, a gas, an organic metal, a drug of abuse or one or more combinations thereof.

Clause 80. The method of any of clauses 64-77, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 81. The method of any of clauses 64-80, wherein the blood sample is whole blood, serum or plasma, either venous blood or capillary blood.

Clause 82. The method of clause 81, wherein the blood sample is obtained from venous blood and is whole blood, serum, or plasma.

Clause 82A. The method of claim 82, wherein the blood sample is a whole blood sample.

Clause 82B. The method of claim 82, wherein the blood sample is serum.

Clause 82C. The method of claim 82, wherein the blood sample is plasma.

Clause 83. The method of clause 81, wherein the blood sample is obtained from capillary blood and is whole blood, serum, or plasma.

Clause 83A. The method of clause 83, wherein the blood sample is a whole blood sample.

Clause 83B. The method of clause 83, wherein the blood sample is serum.

Clause 83C. The method of clause 83, wherein the blood sample is plasma.

Clause 84. The method of any of clauses 64-83, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 84A. The method of clause 84, wherein the blood sample is obtained at one or multiple timepoints after about twenty-five hours.

Clause 84B. The method of clause 84, wherein the blood sample is obtained at one or more multiple timepoints after about thirty hours.

Clause 84C. The method of clause 84, wherein the blood sample is obtained at one or more multiple timepoints after about thirty-five hours.

Clause 84D. The method of clause 84, wherein the blood sample is obtained at one or more multiple timepoints after about forty hours.

Clause 84E. The method of clause 84, wherein the blood sample is obtained at one or more multiple timepoints after about two days.

Clause 84F. The method of clause 84, wherein the blood sample is obtained at one or more multiple timepoints after about five days.

Clause 84G. The method of clause 84, wherein the blood sample is obtained at one or more multiple timepoints after about one week.

Clause 84H. The method of clause 84, wherein the blood sample is obtained at one or more multiple timepoints after about one week.

Clause 841. The method of clause 84, wherein the blood sample is obtained at one or more multiple timepoints after about two weeks.

Clause 84J. The method of clause 84, wherein the blood sample is obtained at one or more multiple timepoints after about three weeks.

Clause 85. The method of any of clauses 64-84, wherein the subject is a human subject.

Clause 86. A method for aiding in the diagnosis and evaluation of a subject that has sustained or may have sustained an acquired brain injury (ABI), the method comprising:
a. performing one or more assays on at least one or more samples obtained from the subject within after about twenty-four hours to (i) about one week, (ii) about two weeks, or (iii) about three weeks, after an actual or suspected ABI to measure or detect a level of a biomarker in the sample, said biomarker comprising a combination of GFAP and UCH-L1;
b. determining that the subject:
i) has not suffered or recovered from an actual ABI if the subject's GFAP level is less than from about 16 pg/mL to about 18 pg/mL and UCH-L1 level is less than from about 49 pg/mL to about 63 pg/mL; or
ii) should be monitored for recovery from an actual ABI if the subject's GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's GFAP level is greater than from about 291 pg/mL to about 431 pg/mL and UCH-L1 level is greater than from about 243 pg/mL to about 275 pg/mL.

Clause 87. The method of clause 86, wherein:
in b i), the subject's GFAP level is less than about 16 pg/mL and UCH-L1 level is less than about 49 pg/mL;
in b ii), the subject's GFAP level is between a range of about 16 pg/mL to about 431 pg/mL and UCH-L1 level is between a range of about 49 pg/mL to about 275 pg/mL; or
in b iii), the subject's GFAP level is greater than about 431 pg/mL and UCH-L1 level is greater than about 275 pg/mL.

Clause 88. The method of clause 86 or clause 87, wherein the GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 88A. The method of clause 86, wherein:
in b i), the GFAP level is less than about 18 pg/mL and the UCH-L1 level is less than about 63 pg/mL;
in b ii), the GFAP level is between about 18 pg/mL at the lower limit up to about 431 pg/mL at the upper limit and the UCH-L1 level is between about 63 pg/mL at the lower limit and about 275 pg/mL at the upper limit; or
in b iii), the GFAP level is greater than about 431 pg/mL and the UCH-L1 level is greater than about 275 pg/mL.

Clause 88B. The method of clause 88A, wherein the GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 89. The method of clause 86, wherein:
in b i), the GFAP level is less than about 16 pg/mL to about 18 pg/mL and the UCH-L1 level is less than about 51 pg/mL or about 55 pg/mL;
in b ii), the GFAP level is between a range of about 16 pg/mL to about 291 pg/mL, about 17 pg/mL to about 307 pg/mL, about 18 pg/mL to about 294 pg/mL and the UCH-L1 level is between about 51 pg/mL to about 248 pg/mL, about 55 pg/mL to about 243 pg/mL, or about 55 pg/mL to about 250 pg/mL; or
in b iii), the GFAP level is greater than about 291 pg/mL, about 294 pg/mL, or about 307 pg/mL and the UCH-L1 level is greater than about 243 pg/mL, about 248 pg/mL, or about 250 pg/mL.

Clause 89A. The method of clause 86, wherein:
in b i), the GFAP level is less than about 18 pg/mL and UCH-L1 level is less than about 55 pg/mL;
in b ii), the GFAP level is between a range of about 18 pg/mL to about 307 pg/mL and the UCH-L1 level is between a range of about 55 pg/mL to about 250 pg/mL; or
in b iii), the GFAP level is greater than about 307 pg/mL and the UCH-L1 level is greater than about 250 pg/mL.

Clause 90. The method of clause 89, wherein (1) b i) is correlated with a level associated with a negative head CT; (2) b ii) is correlated with a positive head CT, a negative head CT, or no head CT; or (3) b iii) is correlated with a positive head CT, a negative head CT or no head CT.

Clause 90A. The method of claim 89A, wherein 1) b i) is correlated with a level associated with a negative head CT; (2) b ii) is correlated with a positive head CT; or (3) b iii) is correlated with a positive head CT.

Clause 91. The method of clause 86, wherein:
in b i), the GFAP level is less than about 17 pg/mL and the UCH-L1 level is less than about 56 pg/mL or about 61 pg/mL;
in b ii), the GFAP level is between a range of about 17 pg/mL to about 415 pg/mL, about 17 pg/mL to about 425 pg/mL, or about 17 pg/mL to about 431 pg/mL and the UCH-L1 level is between about 56 pg/mL to about 250 pg/mL, about 56 pg/mL to about 267 pg/mL, or about 61 pg/mL to about 248 pg/mL; or
in b iii), the GFAP level is greater than about 415 pg/mL, about 425 pg/mL, or about 431 pg/mL and the UCH-L1 level is greater than 248 pg/mL, about 250 pg/mL, or about 267 pg/mL.

Clause 91A. The method of clause 86, wherein:
in b i), the GFAP level is less than about 17 pg/mL and UCH-L1 level is less than about 61 pg/mL;
in b ii), the GFAP level is between a range of about 17 pg/mL to about 431 pg/mL and the UCH-L1 level is between a range of about 61 pg/mL to about 267 pg/mL; or
in b iii), the GFAP level is greater than about 431 pg/mL and the UCH-L1 level is greater than about 267 pg/mL.

Clause 92. The method of clause 91, wherein (1) b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii) is correlated with a GCS score severity for a moderate, severe, or a moderate-to-severe TBI (GCS 3-12).

Clause 92A. The method of claims 91A, wherein (1) b i) is correlated with a level associated with a GCS score severity for a mild TBI (GCS 13-15); (2) b ii) is correlated with a GCS score severity for a moderate, severe, or a moderate-to-severe TBI (GCS 3-12); or (3) b iii) is correlated with a GCS score severity for a moderate, severe, or a moderate-to-severe TBI (GCS 3-12).

Clause 93. The method of clause 86, wherein:
in b i), the GFAP level is less than about 17 pg/mL and the UCH-L1 level is less than about 49 pg/mL, about 53 pg/mL, or about 63 pg/mL;
in b ii), the GFAP level is between a range of about 17 pg/mL to about 375 pg/mL or about 17 pg/mL to about 431 pg/mL and the UCH-L1 level is between about 49 pg/mL to about 275 pg/mL, about 53 pg/mL to about 275 pg/mL, or about 63 pg/mL to about 267 pg/mL; or
in b iii), the GFAP level is greater than about 375 pg/mL or about 431 pg/mL and the UCH-L1 level is greater than about 275 pg/mL or about 267 pg/mL.

Clause 93A. The method of clause 86, wherein:
in b i), the GFAP level is less than about 17 pg/mL and UCH-L1 level is less than about 63 pg/mL;
in b ii), the GFAP level is between a range of about 17 pg/mL to about 431 pg/mL and the UCH-L1 is between a range of about 63 pg/mL to about 275 pg/mL; or
in b iii), the GFAP level is greater than about 431 pg/mL and the UCH-L1 level is greater than about 275 pg/mL.

Clause 94. The method of clause 93, wherein (1) b i) is correlated with a level associated with a GOSE score of 4-8; (2) b ii) is correlated with a level associated with a GOSE score of 0-3; or (3) each of b iii) is correlated with a level associated with a GOSE score of 0-3.

Clause 94A. The method of clause 93A, wherein (1) b i) is correlated with a level associated with a GOSE score of 4-8; (2) b ii) is correlated with a level associated with a GOSE score of 0-3; or (3) each of b iii) (a), b iii) is correlated with a level associated with a GOSE score of 0-3.

Clause 95. The method of any of clauses 86-94, wherein measuring the GFAP level and UCH-L1 level is done by immunoassay or a clinical chemistry assay.

Clause 96. The method of any of clauses 86-95, wherein the assay is a point-of-care assay or single molecule detection.

Clause 97. The method of any of clauses 86-96, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 98. The method of any of clauses 86-97, wherein the actual or suspected ABI is caused by physical shaking, blunt impact by an external mechanical or other force that results in a closed or open head trauma, one or more falls, explosions or blasts or other types of blunt force trauma.

Clause 99. The method of any of clauses 86-99, wherein the actual or suspected ABI is a traumatic brain injury.

Clause 100. The method of any of clauses 86-99, wherein the actual or suspected ABI is caused by fire exposure, ingestion of a chemical or toxin, or a combination of fire exposure, and ingestion of a chemical or toxin.

Clause 101. The method of clause 100, wherein the chemical or toxin is mold, asbestos, a pesticide, an insecticide, an organic solvent, a paint, a glue, a gas, an organic metal, a drug of abuse or one or more combinations thereof.

Clause 102. The method of any of clauses 86-99, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 103. The method of any of clauses 86-102, wherein the blood sample is whole blood, serum or plasma, either venous blood or capillary blood.

Clause 104. The method of clause 103, wherein the blood sample is obtained from venous blood and is whole blood, serum, or plasma.

Clause 104A. The method of claim 104, wherein the blood sample is a whole blood sample.

Clause 104B. The method of claim 104, wherein the blood sample is serum.

Clause 104C. The method of claim 104, wherein the blood sample is plasma.

Clause 105. The method of clause 103, wherein the blood sample is obtained from capillary blood and is whole blood, serum, or plasma.

Clause 105A. The method of clause 105, wherein the blood sample is a whole blood sample.

Clause 105B. The method of clause 105, wherein the blood sample is serum.

Clause 105C. The method of clause 105, wherein the blood sample is plasma.

Clause 106. The method of any of clauses 86-105, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 106A. The method of clause 106, wherein the blood sample is obtained at one or multiple timepoints after about twenty-five hours.

Clause 106B. The method of clause 106, wherein the blood sample is obtained at one or more multiple timepoints after about thirty hours.

Clause 106C. The method of clause 106, wherein the blood sample is obtained at one or more multiple timepoints after about thirty-five hours.

Clause 106D. The method of clause 106, wherein the blood sample is obtained at one or more multiple timepoints after about forty hours.

Clause 106E. The method of clause 106, wherein the blood sample is obtained at one or more multiple timepoints after about two days.

Clause 106F. The method of clause 106, wherein the blood sample is obtained at one or more multiple timepoints after about five days.

Clause 106G. The method of clause 106, wherein the blood sample is obtained at one or more multiple timepoints after about one week.

Clause 106H. The method of clause 106, wherein the blood sample is obtained at one or more multiple timepoints after about one week.

Clause 1061. The method of clause 106, wherein the blood sample is obtained at one or more multiple timepoints after about two weeks.

Clause 106J. The method of clause 106, wherein the blood sample is obtained at one or more multiple timepoints after about three weeks.

Clause 107. The method of any of clauses 86-106, wherein the subject is a human subject.

Clause 108. A system comprising:
an apparatus comprising:
machine readable instructions; and
processor circuitry to at least one of instantiate or execute the machine-readable instructions to:
identify a first biomarker, a second biomarker or a first biomarker and a second biomarkers on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b); and
instructions for determining that the subject:
i) has not suffered or has recovered from an actual acquired brain injury (ABI) if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

Clause 109. The system of clause 108, wherein the apparatus is a point-of-care device or a non-point-of-care device.

Clause 110. An apparatus comprising:
machine readable instructions; and
processor circuitry to at least one of instantiate or execute the machine-readable instructions to:
identify a first biomarker, a second biomarker or a first biomarker and a second biomarkers on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from the apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

Clause 111. The apparatus of clause 110, wherein the apparatus is a point-of-care device or a non-point-of-care device.

Clause 112. A non-transitory machine-readable storage medium comprising instructions to cause one or more processors to at least:
identify a first biomarker, a second biomarker or a first biomarker and a second biomarkers on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from an apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

Clause 113. The non-transitory machine-readable storage medium of clause 112, wherein the apparatus is a point-of-care device or a non-point-of-care device.

Clause 114. A computer-implemented method for evaluation of a subject that has sustained or may have sustained an acquired brain injury (ABI), the method comprising:
a. obtaining data of a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI;
b. analyzing the data to obtain:
i) a first evaluation result if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) a second evaluation result if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) a third evaluation result if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL; and
outputting the evaluation result (e.g., where the evaluation result is the first evaluation result, the second evaluation result, or the third evaluation result),
wherein the first evaluation result is associated with determining that the subject has not suffered or has recovered from an actual ABI; the second evaluation result is associated with determining that the subject should be monitored for recovery from an actual ABI; the third evaluation result is associated with determining that the subject sustained and is not recovering from an actual ABI.

Clause 115. The method of clause 114, wherein:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

Clause 116. The method of clause 114 or clause 115, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 116A. The method of clause 114, wherein:
in b i) (a) the GFAP level is less than about 18 pg/mL; (b) the UCH-L1 level is less than about 63 pg/mL; or (c) the GFAP level is less than about 18 pg/mL and the UCH-L1 level is less than about 63 pg/mL;
in b ii), (a) the GFAP level is between about 18 pg/mL at the lower limit up to about 431 pg/mL at the upper limit; (b) the UCH-L1 level is between about 63 pg/mL at the lower limit and about 275 pg/mL at the upper limit; or (c) the GFAP level is between about 18 pg/mL at the lower limit up to about 431 pg/mL at the upper limit and the UCH-L1 level is between about 63 pg/mL at the lower limit and about 275 pg/mL at the upper limit; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 116B. The method of clause 116A, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (c) any combinations of (a)-(d).

Clause 117. The method of clause 114, wherein:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Clause 117A. The method of clause 114, wherein:
in b i) (a) the GFAP level is less than about 18 pg/mL; (b) UCH-L1 level is less than about 55 pg/mL; or (c) GFAP level is less than about 18 pg/mL and UCH-L1 level is less than about 55 pg/mL;
in b ii), (a) the GFAP level is between a range of about 18 pg/mL to about 307 pg/mL; (b) the UCH-L1 level is between a range of about 55 pg/mL to about 250 pg/mL; or (c) the GFAP level is between a range of about 18 pg/mL to about 307 pg/mL and the UCH-L1 level is between a range of about 55 pg/mL to about 250 pg/mL; or
in b iii), (a) the GFAP level is greater than about 307 pg/mL; (b) the UCH-L1 level is greater than about 250 pg/mL; (c) the GFAP level is greater than about 307 pg/mL and the UCH-L1 level is greater than about 250 pg/mL.

Clause 118. The method of clause 117, wherein (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

Clause 118A. The method of clause 117A, wherein (1) each of b i) (a), b i) (b) and b) i) (c) are correlated with a level associated with a negative head CT; (2) each of b ii) (a), b ii) (b), and b ii) (c) are correlated with a positive head CT; or (3) each of b iii) (a), b iii) (b), and b iii) (c) are correlated with a positive head CT.

Clause 119. The method of clause 114, wherein:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL.

Clause 119A. The method of clause 114, wherein:
in b i) (a) the GFAP level is less than about 17 pg/mL; (b) UCH-L1 level is less than about 61 pg/mL; or (c) GFAP level is less than about 17 pg/mL and UCH-L1 level is less than about 61 pg/mL;
in b ii), (a) the GFAP level is between a range of about 17 pg/mL to about 431 pg/mL; (b) the UCH-L1 level is between a range of about 61 pg/mL to about 267 pg/mL; or (c) the GFAP level is between a range of about 17 pg/mL to about 431 pg/mL and the UCH-L1 level is between a range of about 61 pg/mL to about 267 pg/mL; or
in b iii), (a) the GFAP level is greater than about 431 pg/mL; (b) the UCH-L1 level is greater than about 267 pg/mL; (c) the GFAP level is greater than about 431 pg/mL and the UCH-L1 level is greater than about 267 pg/mL.

Clause 120. The method of clause 119, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 120A. The method of clause 119A, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 121. The method of clause 114, wherein:
in bi) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Clause 121A. The method of clause 114, wherein:
in b i) (a) the GFAP level is less than about 17 pg/mL; (b) UCH-L1 level is less than about 63 pg/mL; or (c) GFAP level is less than about 17 pg/mL and UCH-L1 level is less than about 63 pg/mL;
in b ii), (a) the GFAP level is between a range of about 17 pg/mL to about 431 pg/mL; (b) the UCH-L1 level is between a range of about 63 pg/mL to about 275 pg/mL; or (c) the GFAP level is between a range of about 17 pg/mL to about 431 pg/mL and the UCH-L1 is between a range of about 63 pg/mL to about 275 pg/mL; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 122. The method of clause 121, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 122A. The method of clause 121A, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 123. The method of any of clauses 114-122, wherein measuring the GFAP level, UCH-L1 level, or GFAP level and UCH-L1 level is done by immunoassay or a clinical chemistry assay.

Clause 124. The method of any of clauses 114-123, wherein the assay is performed using a point-of-care assay or single molecule detection.

Clause 125. The method of any of clauses 114-124, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 126. The method of any of clauses 114-125, wherein the actual or suspected ABI is caused by physical shaking, blunt impact by an external mechanical or other force that results in a closed or open head trauma, one or more falls, explosions or blasts or other types of blunt force trauma.

Clause 127. The method of any of clauses 114-126, wherein the actual or suspected ABI is a traumatic brain injury.

Clause 128. The method of any of clauses 114-127, wherein the actual or suspected ABI is caused by fire exposure, ingestion of a chemical or toxin, or a combination of fire exposure, and ingestion of a chemical or toxin.

Clause 129. The method of clause 128, wherein the chemical or toxin is mold, asbestos, a pesticide, an insecticide, an organic solvent, a paint, a glue, a gas, an organic metal, a drug of abuse or one or more combinations thereof.

Clause 130. The method of any of clauses 114-127, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 131. The method of any of clauses 114-130, wherein the blood sample is whole blood, serum or plasma, either venous blood or capillary blood.

Clause 131A. The method of any of clause 131, wherein the blood sample is obtained from venous blood and is whole blood, serum, or plasma.

Clause 131B. The method of claim 131A, wherein the blood sample is a whole blood sample.

Clause 131C. The method of claim 131A, wherein the blood sample is serum.

Clause 131D. The method of claim 131A, wherein the blood sample is plasma.

Clause 131E. The method of clause 131, wherein the blood sample is obtained from capillary blood and is whole blood, serum, or plasma.

Clause 131F. The method of clause 131E, wherein the blood sample is a whole blood sample.

Clause 131G. The method of clause 131E, wherein the blood sample is serum.

Clause 131H. The method of clause 131E, wherein the blood sample is plasma.

Clause 132. The method of any of clauses 114-131, wherein the subject is a human subject.

Clause 133. A system for evaluation of a subject that has sustained or may have sustained an acquired brain injury (ABI), the system comprising:
a. a data-obtaining module to obtain data of a level of glial fibrillary acidic protein (GFAP), a level of ubiquitin carboxy-terminal hydrolase L1 (UCH-L1), or a level of GFAP and a level of UCH-L1 from one or more assays performed on at least one or more samples obtained from a subject from within after about twenty-four hours to (i) about one week, (ii) about two weeks, or (iii) about three weeks, after an actual or suspected ABI;
b. an evaluation module to analyze the data to obtain:
i) a first evaluation result if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) a second evaluation result if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) a third evaluation result if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL; and
c. a data-outputting module to output the evaluation result (e.g., where the evaluation result is the first evaluation result, the second evaluation result, or the third evaluation result),
wherein the first evaluation result is associated with determining that the subject has not suffered or has recovered from an actual ABI; the second evaluation result is associated with determining that the subject should be monitored for recovery from an actual ABI; and the third evaluation result is associated with determining that the subject sustained and is not recovering from an actual ABI.

Clause 134. The system of clause 133, wherein:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

Clause 135. The system of clause 133 or clause 134, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 135A. The system of clause 133, wherein:
in b i) (a) the GFAP level is less than about 18 pg/mL; (b) the UCH-L1 level is less than about 63 pg/mL; or (c) the GFAP level is less than about 18 pg/mL and the UCH-L1 level is less than about 63 pg/mL;
in b ii), (a) the GFAP level is between about 18 pg/mL at the lower limit up to about 431 pg/mL at the upper limit; (b) the UCH-L1 level is between about 63 pg/mL at the lower limit and about 275 pg/mL at the upper limit; or (c) the GFAP level is between about 18 pg/mL at the lower limit up to about 431 pg/mL at the upper limit and the UCH-L1 level is between about 63 pg/mL at the lower limit and about 275 pg/mL at the upper limit; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 135B. The system of clause 135A, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI; (d) a negative CT scan for a TBI; or (e) any combinations of (a)-(d).

Clause 136. The system of clause 133, wherein:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

Clause 136A. The system of clause 133, wherein:
in b i) (a) the GFAP level is less than about 18 pg/mL; (b) UCH-L1 level is less than about 55 pg/mL; or (c) GFAP level is less than about 18 pg/mL and UCH-L1 level is less than about 55 pg/mL;
in b ii), (a) the GFAP level is between a range of about 18 pg/mL to about 307 pg/mL; (b) the UCH-L1 level is between a range of about 55 pg/mL to about 250 pg/mL; or (c) the GFAP level is between a range of about 18 pg/mL to about 307 pg/mL and the UCH-L1 level is between a range of about 55 pg/mL to about 250 pg/mL; or
in b iii), (a) the GFAP level is greater than about 307 pg/mL; (b) the UCH-L1 level is greater than about 250 pg/mL; (c) the GFAP level is greater than about 307 pg/mL and the UCH-L1 level is greater than about 250 pg/mL.

Clause 137. The system of clause 136, wherein (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

Clause 137A. The system of clause 136A, wherein (1) each of b i) (a), b i) (b) and b) i) (c) are correlated with a level associated with a negative head CT; (2) each of b ii) (a), b ii) (b), and b ii) (c) are correlated with a positive head CT; or (3) each of b iii) (a), b iii) (b), and b iii) (c) are correlated with a positive head CT.

Clause 138. The system of clause 133, wherein:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL.

Clause 138A. The system of clause 133, wherein:
in b i) (a) the GFAP level is less than about 17 pg/mL; (b) UCH-L1 level is less than about 61 pg/mL; or (c) GFAP level is less than about 17 pg/mL and UCH-L1 level is less than about 61 pg/mL;
in b ii), (a) the GFAP level is between a range of about 17 pg/mL to about 431 pg/mL; (b) the UCH-L1 level is between a range of about 61 pg/mL to about 267 pg/mL; or (c) the GFAP level is between a range of about 17 pg/mL to about 431 pg/mL and the UCH-L1 level is between a range of about 61 pg/mL to about 267 pg/mL; or
in b iii), (a) the GFAP level is greater than about 431 pg/mL; (b) the UCH-L1 level is greater than about 267 pg/mL; (c) the GFAP level is greater than about 431 pg/mL and the UCH-L1 level is greater than about 267 pg/mL.

Clause 139. The system of clause 138, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 139A. The system of clause 138A, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

Clause 140. The system of clause 133, wherein:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

Clause 140A. The system of clause 133, wherein:
in b i) (a) the GFAP level is less than about 17 pg/mL; (b) UCH-L1 level is less than about 63 pg/mL; or (c) GFAP level is less than about 17 pg/mL and UCH-L1 level is less than about 63 pg/mL;
in b ii), (a) the GFAP level is between a range of about 17 pg/mL to about 431 pg/mL; (b) the UCH-L1 level is between a range of about 63 pg/mL to about 275 pg/mL; or (c) the GFAP level is between a range of about 17 pg/mL to about 431 pg/mL and the UCH-L1 is between a range of about 63 pg/mL to about 275 pg/mL; or
in b iii), (a) the GFAP level is greater than about 431 pg/mL; (b) the UCH-L1 level is greater than about 275 pg/mL; (c) the GFAP level is greater than about 431 pg/mL and the UCH-L1 level is greater than about 275 pg/mL.

Clause 141. The system of clause 140, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 141A. The system of clause 140A, (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

Clause 142. The system of any of clauses 133-141, wherein measuring the GFAP level, UCH-L1 level, or GFAP level and UCH-L1 level is done by immunoassay or a clinical chemistry assay.

Clause 143. The system of any of clauses 133-142, wherein the assay is performed using a point-of-care assay or single molecule detection.

Clause 144. The system of any of clauses 133-143, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 145. The system of any of clauses 133-144, wherein the actual or suspected ABI is caused by physical shaking, blunt impact by an external mechanical or other force that results in a closed or open head trauma, one or more falls, explosions or blasts or other types of blunt force trauma.

Clause 146. The system of any of clauses 133-145, wherein the actual or suspected ABI is a traumatic brain injury.

Clause 147. The system of any of clauses 133-146, wherein the actual or suspected ABI is caused by fire exposure, ingestion of a chemical or toxin, or a combination of fire exposure, and ingestion of a chemical or toxin.

Clause 148. The system of clause 147, wherein the chemical or toxin is mold, asbestos, a pesticide, an insecticide, an organic solvent, a paint, a glue, a gas, an organic metal, a drug of abuse or one or more combinations thereof.

Clause 149. The system of any of clauses 133-146, wherein the actual or suspected ABI is caused by an autoimmune disease, a metabolic disorder, a brain tumor, hypoxia, a viral infection, a fungal infection, a bacterial infection, meningitis, hydrocephalus, or any combinations thereof.

Clause 150. The system of any of clauses 133-149, wherein the blood sample is whole blood, serum or plasma, either venous blood or capillary blood.

Clause 150A. The system of any of clause 150, wherein the blood sample is obtained from venous blood and is whole blood, serum, or plasma.

Clause 150B. The system of claim 150A, wherein the blood sample is a whole blood sample.

Clause 150C. The system of claim 150A, wherein the blood sample is serum.

Clause 150D. The system of claim 150A, wherein the blood sample is plasma.

Clause 150E. The system of clause 150, wherein the blood sample is obtained from capillary blood and is whole blood, serum, or plasma.

Clause 150F. The system of clause 150E, wherein the blood sample is a whole blood sample.

Clause 150G. The system of clause 150E, wherein the blood sample is serum.

Clause 150H. The system of clause 150E, wherein the blood sample is plasma.

Clause 151. The system of any of clauses 133-150, wherein the subject is a human subject.

Clause 152. A computer device comprising a storage device having machine-readable instructions stored thereon, and a processor, wherein the processor executes the machine-readable instructions to perform steps of the method of any one of clauses 114-132 or steps performed by the system of any one of clauses 133-151.

Clause 153. A non-transitory machine-readable storage medium having machine-readable instructions stored thereon, wherein the machine-readable instructions are configured to be executed by a processor to perform steps of the method of any one of clauses 114-132 or steps performed by the system of any one of clauses 133-151.

Clause 154. A system comprising:
an apparatus comprising:
a storage device having machine-readable instructions stored thereon; and
a processor to at least one of instantiate or execute the machine-readable instructions to:
identify a first biomarker, a second biomarker or a first biomarker and a second biomarkers on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b); and
instructions for determining that the subject:
i) has not suffered or has recovered from an actual acquired brain injury (ABI) if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

Clause 155. The system of clause 154, wherein the apparatus is a point-of-care device or a non-point-of-care device.

Clause 156. An apparatus comprising:
a storage device having machine-readable instructions stored on the storage device; and and
a processor to at least one of instantiate or execute the machine-readable instructions to:
identify a first biomarker, a second biomarker or a first biomarker and a second biomarkers on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from the apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

Clause 157. The apparatus of clause 156, wherein the apparatus is a point-of-care device or a non-point-of-care device.

Clause 158. The method of any of clauses 1-19 or clauses 114-132, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 158A. The method of clause 158, wherein the blood sample is obtained at one or multiple timepoints after about twenty-five hours.

Clause 158B. The method of clause 158, wherein the blood sample is obtained at one or more multiple timepoints after about thirty hours.

Clause 158C. The method of clause 158, wherein the blood sample is obtained at one or more multiple timepoints after about thirty-five hours.

Clause 158D. The method of clause 158, wherein the blood sample is obtained at one or more multiple timepoints after about forty hours.

Clause 158E. The method of clause 158, wherein the blood sample is obtained at one or more multiple timepoints after about two days.

Clause 158F. The method of clause 158, wherein the blood sample is obtained at one or more multiple timepoints after about five days.

Clause 158G. The method of clause 158, wherein the blood sample is obtained at one or more multiple timepoints after about one week.

Clause 158H. The method of clause 158, wherein the blood sample is obtained at one or more multiple timepoints after about one week.

Clause 1581. The method of clause 158, wherein the blood sample is obtained at one or more multiple timepoints after about two weeks.

Clause 158J. The method of clause 158, wherein the blood sample is obtained at one or more multiple timepoints after about three weeks.

Clause 159. The system of any of clauses 108-109, clauses 133-151 or clauses 154-155, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 159A. The system of clause 159, wherein the blood sample is obtained at one or multiple timepoints after about twenty-five hours.

Clause 159B. The system of clause 159, wherein the blood sample is obtained at one or more multiple timepoints after about thirty hours.

Clause 159C. The system of clause 159, wherein the blood sample is obtained at one or more multiple timepoints after about thirty-five hours.

Clause 159D. The system of clause 159, wherein the blood sample is obtained at one or more multiple timepoints after about forty hours.

Clause 159E. The system of clause 159, wherein the blood sample is obtained at one or more multiple timepoints after about two days.

Clause 159F. The system of clause 159, wherein the blood sample is obtained at one or more multiple timepoints after about five days.

Clause 159G. The system of clause 159, wherein the blood sample is obtained at one or more multiple timepoints after about one week.

Clause 159H. The system of clause 159, wherein the blood sample is obtained at one or more multiple timepoints after about one week.

Clause 1591. The system of clause 159, wherein the blood sample is obtained at one or more multiple timepoints after about two weeks.

Clause 159J. The system of clause 159, wherein the blood sample is obtained at one or more multiple timepoints after about three weeks.

Clause 160. The apparatus of any of clauses 110-111 or clauses 156-157, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 160A. The apparatus of clause 160, wherein the blood sample is obtained at one or multiple timepoints after about twenty-five hours.

Clause 160B. The apparatus of clause 160, wherein the blood sample is obtained at one or more multiple timepoints after about thirty hours.

Clause 160C. The apparatus of clause 160, wherein the blood sample is obtained at one or more multiple timepoints after about thirty-five hours.

Clause 160D. The apparatus of clause 160, wherein the blood sample is obtained at one or more multiple timepoints after about forty hours.

Clause 160E. The apparatus of clause 160, wherein the blood sample is obtained at one or more multiple timepoints after about two days.

Clause 160F. The apparatus of clause 160, wherein the blood sample is obtained at one or more multiple timepoints after about five days.

Clause 160G. The apparatus of clause 160, wherein the blood sample is obtained at one or more multiple timepoints after about one week.

Clause 160H. The apparatus of clause 160, wherein the blood sample is obtained at one or more multiple timepoints after about one week.

Clause 1601. The apparatus of clause 160, wherein the blood sample is obtained at one or more multiple timepoints after about two weeks.

Clause 160J. The apparatus of clause 160, wherein the blood sample is obtained at one or more multiple timepoints after about three weeks.

Clause 161. The non-transitory machine-readable medium of any of clauses 112-113, wherein the blood sample is obtained at one or multiple timepoints within after about twenty-four hours, after about twenty-five hours, after about twenty-six hours, after about twenty-seven hours, after about twenty-eight hours, after about twenty-nine hours, after about thirty hours, after about thirty-five hours, after about thirty-six hours, after about thirty-seven hours, after about thirty-eight hours, after about thirty-nine hours, after about forty hours, after about two days, after about three days, after about four days, after about five days, after about six days one week, about eight days, about nine days, about ten days, about eleven days, about twelve days, about thirteen days, about two weeks, about fifteen days, about sixteen days, about seventeen days, about eighteen days, about nineteen days, about twenty days, or about three weeks after an actual or suspected ABI in the subject.

Clause 161A. The non-transitory machine-readable medium of clause 161, wherein the blood sample is obtained at one or multiple timepoints after about twenty-five hours.

Clause 161B. The non-transitory machine-readable medium of clause 161, wherein the blood sample is obtained at one or more multiple timepoints after about thirty hours.

Clause 161C. The non-transitory machine-readable medium of clause 161, wherein the blood sample is obtained at one or more multiple timepoints after about thirty-five hours.

Clause 161D. The non-transitory machine-readable medium of clause 161, wherein the blood sample is obtained at one or more multiple timepoints after about forty hours.

Clause 161E. The non-transitory machine-readable medium of clause 161, wherein the blood sample is obtained at one or more multiple timepoints after about two days.

Clause 161F. The non-transitory machine-readable medium of clause 161, wherein the blood sample is obtained at one or more multiple timepoints after about five days.

Clause 161G. The non-transitory machine-readable medium of clause 161, wherein the blood sample is obtained at one or more multiple timepoints after about one week.

Clause 161H. The non-transitory machine-readable medium of clause 161, wherein the blood sample is obtained at one or more multiple timepoints after about one week.

Clause 161I. The non-transitory machine-readable medium of clause 161, wherein the blood sample is obtained at one or more multiple timepoints after about two weeks.

Clause 161J. The non-transitory machine-readable medium of clause 161, wherein the blood sample is obtained at one or more multiple timepoints after about three weeks.

Clause 162. The method of any of clauses 1-107 or clauses 114-132, wherein the subject was stratified based on whether the subject was: (a) evaluated in an emergency department and subsequently discharged; (b) admitted to a hospital, but not to an intensive or critical care unit; or (c) admitted to a hospital and further admitted to an intensive care or critical care unit.

Clause 163. The method of any of clauses 1-107 or clauses 114-132, wherein the ABI is a subacute ABI.

Clause 164. The method of any of clauses 14, 33, 55, 77, 99 or 127, wherein the traumatic brain injury is a subacute TBI.

1. A method for aiding in the diagnosis and evaluation of a subject that has sustained or may have sustained an acquired brain injury (ABI), the method comprising:
a. performing one or more assays on at least one or more samples obtained from the subject within after about twenty-four hours to (i) about one week, (ii) about two weeks, or (iii) about three weeks, after an actual or suspected ABI to measure or detect a level of a biomarker in the sample, said biomarker comprising (i) a combination of glial fibrillary acidic protein (GFAP) and ubiquitin carboxy-terminal hydrolase L1 (UCH-L1), (ii) GFAP, or (iii) UCH-L1;
b. determining that the subject:
i) has not suffered or recovered from an actual ABI if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

2. The method of claim 1, wherein:
in b i): the subject's: (a) GFAP level is less than about 16 pg/mL; (b) UCH-L1 level is less than about 49 pg/mL; or (c) GFAP level is less than about 16 pg/mL and UCH-L1 level is less than about 49 pg/mL;
in b ii), the subject's: (a) GFAP level is between a range of about 16 pg/mL to about 431 pg/mL; (b) UCH-L1 level is between a range of about 49 pg/mL to about 275 pg/mL; or (c) GFAP level is between a range of about 16 pg/mL to about 431 pg/mL and UCH-L1 level is between a range of about 49 pg/mL to about 275 pg/mL; or
in b iii) the subject's: (a) GFAP level is greater than about 431 pg/mL; (b) UCH-L1 level is greater than about 275 pg/mL; or (c) GFAP level is greater than about 431 pg/mL and UCH-L1 level is greater than about 275 pg/mL.

3. The method of claim 1, wherein the GFAP level, UCH-L1 level, or GFAP and UCH-L1 level is correlated with a level associated with: (a) a subject's Glasgow Coma Scale Score; (b) a subject's Extended Glasgow Outcome Scale Score; (c) a positive CT scan for a TBI;
(d) a negative CT scan for a TBI; or (e) any combinations of (a)-(d).

4. The method of claim 1, wherein:
in b i) (a) the GFAP level is less than from about 16 pg/mL to about 18 pg/mL; (b) UCH-L1 level is less than about 51 pg/mL or about 55 pg/mL; or (c) GFAP level is less than about 16 pg/mL to about 18 pg/mL and UCH-L1 level is less than about 51 pg/mL or about 55 pg/mL;
in b ii), (a) the GFAP level is between a range of about 16 pg/mL to about 291 pg/mL, about 17 pg/mL to about 307 pg/mL, about 18 pg/mL to about 294 pg/mL; (b) the UCH-L1 level is between about 51 pg/mL to about 248 pg/mL, about 55 pg/mL to about 243 pg/mL, or about 55 pg/mL to about 250 pg/mL; or (c) any combination of one of (a) and one of (b); or
in b iii), (a) the GFAP level is greater than about 291 pg/mL, about 294 pg/mL, or about 307 pg/mL; (b) the UCH-L1 level is greater than about 243 pg/mL, about 248 pg/mL, or about 250 pg/mL; (c) the GFAP level is greater than about 291 pg/mL, about 294 pg/mL, or about 307 pg/mL, and the UCH-L1 level is greater than about 243 pg/mL, about 248 pg/mL, or about 250 pg/mL.

5. The method of claim 4, wherein (1) each of b i) (a), b i) (b) and b) i) (c) are correlated with a level associated with a negative head CT; (2) each of b ii) (a), b ii) (b), and b ii) (c) are correlated with a positive head CT, a negative head CT, or no head CT; or (3) each of b iii) (a), b iii) (b), and b iii) (c) are correlated with a positive head CT, a negative head CT or no head CT.

6. The method of claim 1, wherein:
in b i) (a) the GFAP level is less than from about 17 pg/mL; (b) UCH-L1 level is less than about 56 pg/mL or about 61 pg/mL; or (c) GFAP level is less than about 17 pg/mL and UCH-L1 level is less than about 56 pg/mL or about 61 pg/mL;
in b ii), (a) the GFAP level is between a range of about 17 pg/mL to about 415 pg/mL, about 17 pg/mL to about 425 pg/mL, or about 17 pg/mL to about 431 pg/mL; (b) the UCH-L1 level is between about 56 pg/mL to about 250 pg/mL, about 56 pg/mL to about 267 pg/mL, or about 61 pg/mL to about 248 pg/mL; or (c) any combination of one of (a) and one of (b); or
in b iii), (a) the GFAP level is greater than about 415 pg/mL, about 425 pg/mL, or about 431 pg/mL; (b) the UCH-L1 level is greater than about 248 pg/mL, about 250 pg/mL, or about 267 pg/mL; (c) the GFAP level is greater than about 415 pg/mL, about 425 pg/mL, or about 431 pg/mL and the UCH-L1 level is greater than 248 pg/mL, about 250 pg/mL, or about 267 pg/mL.

7. The method of claim 6, wherein (1) each of b i) (a), b i) (b) and b) i) (c) are correlated with a level associated with a GCS score severity for a mild TBI (GCS 13-15); (2) each of b ii) (a), b ii) (b), and b ii) (c) are correlated with a GCS score severity for a moderate, severe, or a moderate-to-severe TBI (GCS 3-12); or (3) each of b iii) (a), b iii) (b), and b iii) (c) are correlated with a GCS score severity for a moderate, severe, or a moderate-to-severe TBI (GCS 3-12).

8. The method of claim 1, wherein:
in b i) (a) the GFAP level is less than from about 17 pg/mL; (b) UCH-L1 level is less than about 49 pg/mL, about 53 pg/mL, or about 63 pg/mL; or (c) GFAP level is less than about 17 pg/mL and UCH-L1 level is less than about 49 pg/mL, about 53 pg/mL, or about 63 pg/mL;
in b ii), (a) the GFAP level is between a range of about 17 pg/mL to about 375 pg/mL or about 17 pg/mL to about 431 pg/mL; (b) the UCH-L1 level is between about 49 pg/mL to about 275 pg/mL, about 53 pg/mL to about 275 pg/mL, or about 63 pg/mL to about 267 pg/mL; or (c) any combination of one of (a) and one of (b); or
in b iii), (a) the GFAP level is greater than about 375 pg/mL or about 431 pg/mL; (b) the UCH-L1 level is greater than about 275 pg/mL or about 267 pg/mL; (c) the GFAP level is greater than about 375 pg/mL or about 431 pg/mL and the UCH-L1 level is greater than about 275 pg/mL or about 267 pg/mL.

9. The method of claim 8, wherein (1) each of b i) (a), b i) (b) and b) i) (c) are correlated with a level associated with a GOSE score of 4-8; (2) each of b ii) (a), b ii) (b), and b ii) (c) are correlated with a level associated with a GOSE score of 0-3; or (3) each of b iii) (a), b iii) (b), and b iii) (c) are correlated with a level associated with a GOSE score of 0-3.

10. The method of claim 1, wherein measuring the GFAP level, UCH-L1 level, or GFAP level and UCH-L1 level is done by immunoassay or a clinical chemistry assay.

11. The method of claim 1, wherein the assay is a point-of-care assay or single molecule detection assay.

12. The method of claim 1, wherein the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

13. The method of claim 1, wherein the actual or suspected ABI is:
(a) caused by physical shaking, blunt impact by an external mechanical or other force that results in a closed or open head trauma, one or more falls, explosions or blasts or other types of blunt force trauma;
(b) caused by fire exposure, ingestion of a chemical or toxin, or a combination of fire exposure, and ingestion of a chemical or toxin; or
(c) by an autoimmune disease, a metabolic disorder, a brain tumor, hypoxia, a viral infection, a fungal infection, a bacterial infection, meningitis, hydrocephalus, or any combinations thereof.

14. The method of claim 1, wherein the actual or suspected ABI is a traumatic brain injury.

15. (canceled)

16. The method of claim 13, wherein the chemical or toxin is mold, asbestos, a pesticide, an insecticide, an organic solvent, a paint, a glue, a gas, an organic metal, a drug of abuse or one or more combinations thereof.

17. (canceled)

18. The method of claim 1, wherein the blood sample is whole blood, serum or plasma, either venous blood or capillary blood.

19. The method of claim 1, wherein the subject is a human subject.

20. A system comprising:
an apparatus comprising:
a storage device having machine-readable instructions stored thereon; and
a processor to at least one of instantiate or execute the machine-readable instructions to:
identify a (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii);
or a combination of (a) and (b); and
instructions for determining that the subject:
i) has not suffered or has recovered from an actual acquired brain injury (ABI) if the subject's: (a) GFAP level is less than from about 16 pg/mL to about 18 pg/mL; (b) UCH-L1 level is less than from about 49 pg/mL to about 63 pg/mL; or (c) GFAP level is less than from about 16 pg/mL to about 18 pg/mL and UCH-L1 level is less than from about 49 pg/mL to about 63 pg/mL;
ii) should be monitored for recovery from an actual ABI if the subject's: (a) GFAP level is between a range of from about 16 pg/mL to about 18 pg/mL at the lower limit up to from about 291 pg/mL to about 431 pg/mL at the upper limit; (b) UCH-L1 level is between a range of from about 49 pg/mL to about 63 pg/mL at the lower limit up to from about 243 pg/mL to about 275 pg/mL at the upper limit; or (c) GFAP level is between a range of from about 16 pg/mL to about 18 pg/mL at the lower limit up to from about 291 pg/mL to about 431 pg/mL at the upper limit and UCH-L1 level is between a range of from about 49 pg/mL to about 63 pg/mL at the lower limit up to from about 243 pg/mL to about 275 pg/mL at the upper limit; or
iii) sustained and is not recovering from an actual ABI if the subject's: (a) GFAP level is greater than from about 291 pg/mL to about 431 pg/mL; (b) UCH-L1 level is greater than from about 243 pg/mL to about 275 pg/mL; or (c) GFAP level is greater than from about 291 pg/mL to about 431 pg/mL and UCH-L1 level is greater than from about 243 pg/mL to about 275 pg/mL.

21. The system of claim 20, wherein the apparatus is a point-of-care device or a non-point-of-care device.

22. An apparatus comprising:
a storage device having machine-readable instructions stored on the storage device; and
a processor to at least one of instantiate or execute the machine-readable instructions to:
identify (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from the apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

23. The apparatus of claim 22, wherein the apparatus is a point-of-care device or a non-point-of-care device.

24. A non-transitory machine-readable storage medium comprising instructions to cause one or more processors to at least:
identify (i) a first biomarker and a second biomarker, (ii) a first biomarker, or (iii) a second biomarker on at least one or more samples obtained from a subject using at least one assay, wherein the first biomarker is glial fibrillary acidic protein (GFAP) and the second biomarker is ubiquitin carboxy-terminal hydrolase L1 (UCH-L1), wherein the at least one or more samples is obtained from the subject within after about twenty-four hours to (i) about one week, (ii) about two weeks, or (iii) about three weeks, after an actual or suspected ABI;
determine a level of GFAP in the one or more samples, determine a level of UCH-L1 in one or more samples, or determine a level of GFAP in one or more samples and a level of UCH-L1 in one or more samples, and
communicate from an apparatus whether the level of (a) GFAP is greater than, less than or between a reference level of (i) from about 16 pg/mL to about 18 pg/mL, (ii) between a range of from about 16 pg/mL to about 18 pg/mL at the lower limit up to from about 291 pg/mL to about 431 pg/mL at the upper limit; (iii) from about 291 pg/mL to about 431 pg/mL, or (iv) a combination of (i)-(iii); (b) UCH-L1 is above, below, or between a reference level of (i) from about 49 pg/mL to about 63 pg/mL, (ii) between a range of from about 49 pg/mL to about 63 pg/mL at the lower limit up to from about 243 pg/mL to about 275 pg/mL at the upper limit; (iii) from about 243 pg/mL to about 275 pg/mL, or (iv) a combination of (i)-(iii); or a combination of (a) and (b).

25. The non-transitory machine-readable storage medium of claim 24, wherein the apparatus is a point-of-care device or a non-point-of-care device.


SEQUENCE LISTING (5 sequences)

SEQ ID NO: 1 - protein, 223 aa, synthetic construct [sequence omitted]

SEQ ID NO: 2 - protein, 432 aa, synthetic construct
  REGION 1..432: note = Synthetic
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 3 - protein, 6 aa, synthetic construct
  REGION 1..6: note = Synthetic
HHHHHH

SEQ ID NO: 4 - protein, 5 aa, synthetic construct
  REGION 1..5: note = Synthetic
DDDDK

SEQ ID NO: 5 - protein, 6 aa, synthetic construct
  REGION 1..6: note = Synthetic
ADDDDK